(19) United States
(12) Patent Application Publication
(10) Pub. No.: US 2013/0225431 A1
(43) Pub. Date: Aug. 29, 2013

(54) ASSESSMENT OF CELLULAR FRAGMENTATION DYNAMICS FOR DETECTION OF HUMAN EMBRYONIC ANEUPLOIDY

(71) Applicant: The Board of Trustees of the Leland Stanford Junior University, (US)

(72) Inventors: Shawn L. Chavez, Fremont, CA (US); Jinnuo Han, Mountain View, CA (US); Renee A. Reijo Pera, Los Altos, CA (US)

(73) Assignee: The Board of Trustees of the Leland Stanford Junior University, Palo Alto, CA (US)

(21) Appl. No.: 13/773,361

(22) Filed: Feb. 21, 2013

Related U.S. Application Data
(60) Provisional application No. 61/716,506, filed on Oct. 20, 2012.
(60) Provisional application No. 61/602,275, filed on Feb. 23, 2012.

(51) Int. Cl. C12Q 1/68

(52) U.S. Cl. CPC C12Q 1/6813
USPC 506/9; 435/29; 435/6.11; 435/7.1

(57) ABSTRACT

Methods of evaluating the developmental potential of human embryos are disclosed. In particular, the invention relates to methods of detecting embryonic aneuploidy by assessment of cellular fragmentation dynamics in individual blastomeres of embryos up to the 4-cell stage. The methods of the invention should improve in vitro fertilization (IVF) outcomes by reducing inadvertent transfer of non-viable embryos likely to result in spontaneous miscarriage.

### CROSS-REFERENCE TO RELATED APPLICATION

This application claims benefit under 35 U.S.C. §119(e) of provisional application 61/716,506, filed Oct. 20, 2012 and provisional application 61/602,275, filed Feb. 23, 2012, which applications are hereby incorporated by reference in their entireties.

### TECHNICAL FIELD

The present invention pertains generally to methods of evaluating the developmental potential of human embryos. In particular, the invention relates to methods of detecting embryonic aneuploidy by assessment of cellular fragmentation dynamics.

### BACKGROUND

Approximately 10-15% of couples of reproductive age are infertile, and many of these couples opt to use in vitro fertilization (IVF) in order to conceive. According to the Centers for Disease Control and Prevention, the number of couples seeking IVF is on the rise and may continue to increase if the general population continues to postpone having children (cdc.gov/art). In the United States alone, it is estimated that over 1 million human embryos are produced annually for IVF, often with variable and poorly defined potential for successful implantation and development to term. Indeed, the average live birth rate per cycle following IVF was reported to be 30%, and this percentage has only moderately increased since the introduction of IVF more than 30 years ago (Stern et al. (2007) Fertil. Steril. 882:75-282). Besides modest IVF success rates, approximately 30% of live births are multiple gestations, which have well-documented adverse outcomes for both the mother and fetuses such as increased risk of miscarriage, pre-term birth, low birth rate and the necessity for fetal reduction in certain instances (cdc.gov/art). Clearly, the transfer of multiple embryos with variable potential is paradoxically associated with both high rates of embryonic loss and increased incidence of multiples births in IVF (Racowsky (2002) Theriogenology 57: 87-96).

Under current clinical practices, developmental competence of human embryos is typically assessed either on Day 3 or Day 5 based on relatively simple morphological characteristics that may include blastomere number and symmetry or asymmetry and a relative measure of the degree of cellular fragmentation at a single time point, most typically on Day 3 (Antczak et al. (1999) Hum. Reprod. 14:429-447; Alikani et al. (1999) Fertil. Steril. 71:836-842; Ebner et al. (2001) Fertil. Steril. 76:281-285; and Racowsky et al. (2011) Fertil. Steril. 95:1985-1989). The relative measure of fragmentation is typically expressed as no fragmentation, 10%, 25%, and greater than 50% fragmentation. Cellular fragmentation, or the generation of what is thought to be anucleated cytoplasmic fragments, is distinct from the DNA fragmentation that can occur following cell death late in pre-implantation development (Hardy (1999) Rev. Reprod. 4:125-134; Hardy et al. (2001) Proc. Natl. Acad. Sci. U.S.A. 98:1655-1660; and Xu et al. (2001) Fertil. Steril. 75:986-991). While a typical occurrence in human embryos (Antczak et al., supra), cellular fragmentation is observed to a lesser extent in bovine embryos (Sugimura et al. (2010) Biol. Reprod. 83:970-978) and occurs much less frequently in murine embryos (Han et al. (2010) Am. J. Physiol. Cell. Physiol. 298:C1235-1244). There is also evidence to suggest that cellular fragmentation occurs in human embryos in vivo, indicating that fragmentation is not only a consequence of in vitro culture (Pereda et al. (1978) Biol. Reprod. 18:481-489; Buster et al. (1985) Am. J. Obstet. Gynecol. 153:211-217) and may be negatively correlated with implantation potential (Alikani et al. (1999) Fertil. Steril. 71:836-842; Pelinck et al. (2010) Fertil. Steril. 94:527-534).

Although the possible causes of implantation or pregnancy failure are likely to be diverse, it is thought that abnormalities in chromosome number (aneuploidy) are the major contributors to nominal IVF success and live birth rates (Munne et al. (2003) Reprod. Biomed. Online 7: 91-97). Previous studies using whole genome approaches have also demonstrated that chromosomal aneuploidies are remarkably common, occurring in as many as 50-80% of cleavage-stage human embryos, including those from fertile couples (Johnson et al. (2010) Hum. Reprod. 25:1066-1075; Vanneste et al. (2009) Nat. Med. 15: 577-583). This is in sharp contrast to the frequency of aneuploidies in several other species, including the mouse, which typically exhibit less than 1% embryonic aneuploidy rates at this stage of development (Lightfoot et al. (2006) Dev. Biol. 289:384-394). The incidence of human embryonic aneuploidy observed in vitro is thought to reflect that in vivo given that approximately 30% of natural human conceptions result in live births, and chromosomal abnormalities have been reported in as many as 70% of spontaneous miscarriage cases (Macklon et al. (2002) Hum. Reprod. Update 8:333-343; Causio et al. (2002) Eur. J. Obstet. Gynecol. Reprod. Biol. 105:44-48; Spandorfer et al. (2004) Fertil. Steril. 81:1265-1269; Lathi et al. (2004) Fertil. Steril. 81:1270-1272; Bettio et al. (2008) Placenta 29 Suppl B:126-128; and Kushnir (2009) J. Assist. Reprod. Genet. 26:93-97); Kim et al. (2010) BMC Med. Genet. 11:153). It is thought that chromosomal errors most commonly occur on the meiotic spindle during oogenesis, whereas other errors occur less frequently on the mitotic spindle during embryonic cleavage divisions. Such errors could be perpetuated due to the apparent lack of cell cycle checkpoints in cleaving human embryos (Harrison et al. (2000) Zygote 8:217-224). However, other studies have indicated that chromosomal abnormalities in human embryos at the cleavage stage can be complex and may be incongruent with this notion, suggesting that alternative mechanisms distinct from those involving the meiotic or mitotic spindle may account for complex alterations in chromosomal composition (Johnson et al. (2010) Hum. Reprod. 25:1066-1075; Vanneste et al. (2009) Nat. Med. 15: 577-583).

While attempts have been made to correlate morphology with aneuploidy, it is well-known that aneuploid embryos often appear normal and suitable for transfer under traditional IVF assessment techniques that focus on distinct snapshots during development (Baltaci et al. (2006) Reprod. Biomed. Online 12:77-82). Currently, the most frequently used method for diagnosing aneuploidy is pre-implantation genetic screening (PGS) of day 3 biopsied blastomeres, which is invasive to the embryo, suffers from mosaicism and is utilized by only a small proportion of assisted reproduction patients (Kuo et al. (1998) J. Assist. Reprod. Genet. 15:276-280; Baart et al. (2006) Hum. Reprod. 21:223-233). Alternative approaches such as extended culture of embryos to the blastocyst stage and analysis of chromosomal status via trophoectoderm biopsy have also been used more recently to evaluate aneuploidy (Schoolcraft et al. (2011) Fertil. Steril. 96:638-640). However, additional potential risks are associated with prolonged embryo culture and include the introduction of epigenetic changes, embryo arrest, and other factors that disrupt embryo integrity, such as monozygotic twinning. Moreover, trophectoderm biopsy and analysis requires that embryo transfer be delayed until chromosomal testing is completed (Khosla et al. (2001) Hum. Reprod. Update 7:419-427; Katari et al. (2009) Hum. Mol. Genet. 18:3769-3778; Lim et al. (2009) Hum. Reprod. 24: 741-747; and Fernandez-Gonzalez et al. (2009) Reproduction 137:271-283).

Thus, there remains a need for more reliable and less invasive approaches for detection of aneuploidy in human embryos that will allow selection of viable embryos for transfer following IVF.

### SUMMARY

The invention relates to methods of evaluating the developmental potential of human embryos. In particular, the invention relates to methods of detecting embryonic aneuploidy by assessment of cellular fragmentation dynamics in individual blastomeres of embryos up to the 4-cell stage. The methods of the invention should improve in vitro fertilization (IVF) outcomes by avoiding inadvertent transfer of non-viable embryos likely to result in miscarriage.

In one aspect, the invention includes a method for detecting aneuploidy in an embryo. The method comprises (i) observing the embryo optically during development from the 1-cell stage to the 4-cell stage, wherein observations of the embryo are made periodically at time intervals of no more than 30 minutes; and (ii) detecting embryonic micronuclei or cellular fragmentation if present, wherein the presence of embryonic micronuclei or cellular fragmentation indicates the embryo is aneuploid.

More specifically, the timing of the appearance of embryonic micronuclei or cellular fragmentation during development of the embryo can be correlated with the type of aneuploidy and the severity of chromosomal errors. Embryos with meiotic errors and triploid embryos typically have embryonic micronuclei or exhibit fragmentation beginning at the 1-cell stage prior to the first cytokinesis. In contrast, embryos with mitotic errors typically have embryonic micronuclei or exhibit fragmentation following the division of 1-cell to 2-cells or later in development at the 3-cell or 4-cell stage. Accordingly, the timing of the appearance of embryonic micronuclei or cellular fragmentation can be correlated with the developmental potential of the embryo.

Thus, in one aspect, the invention includes a method for evaluating the developmental potential of an embryo comprising observing the embryo during development from the 1-cell stage to the 4-cell stage optically, wherein observations of the embryo are made periodically at time intervals of no more than 30 minutes, and detecting embryonic micronuclei or cellular fragmentation if present, wherein the timing of the appearance of the embryonic micronuclei or cellular fragmentation is correlated with the developmental potential of the embryo. In one embodiment, the appearance of embryonic micronuclei or cellular fragmentation at the 1-cell stage is correlated with the likelihood of embryonic lethality arising from meiotic chromosomal errors. In another embodiment, the appearance of embryonic micronuclei or cellular fragmentation at the 2-cell, 3-cell, or 4-cell stage is correlated with the likelihood of embryonic lethality arising from mitotic chromosomal errors.

In another aspect, the invention includes a method for selecting a human embryo with favorable developmental potential for transfer to a female subject. In one embodiment, the method comprises (a) culturing an embryo under conditions suitable for embryonic development, (b) observing the embryo during development from the 1-cell stage to the 4-cell stage optically, wherein observations of the embryo are made periodically at time intervals of no more than 30 minutes, and (c) selecting an embryo for transfer to the female subject, wherein the embryo has no detectable formation of embryonic micronuclei or cellular fragmentation during development from the 1-cell stage to the 4-cell stage.

In another embodiment, the method comprises (a) culturing one or more embryos under conditions suitable for embryonic development; (b) observing the embryo during development from the 1-cell stage to the 4-cell stage optically, wherein observations of the embryo are made periodically at time intervals of no more than 30 minutes; and (c) selecting an embryo for transfer to the female subject, wherein the embryo has no detectable cellular fragmentation at the 2-cell stage, 3-cell stage, or 4-cell stage in order to avoid transfer of an embryo with mitotic chromosomal errors.

In another embodiment, the method comprises (a) culturing one or more embryos under conditions suitable for embryonic development; (b) observing the embryo during development from the 1-cell stage to the 4-cell stage optically, wherein observations of the embryo are made periodically at time intervals of no more than 30 minutes; and (c) selecting an embryo for transfer to the female subject, wherein the embryo has no detectable cellular fragmentation at the 1-cell stage in order to avoid transfer of an embryo with meiotic chromosomal errors.

In another embodiment, the method comprises (a) culturing one or more embryos under conditions suitable for embryonic development; (b) observing the embryo during development from the 1-cell stage to the 4-cell stage optically, wherein observations of the embryo are made periodically at time intervals of no more than 30 minutes; and (c) selecting an embryo for transfer to the female subject, wherein the embryo has no detectable formation of embryonic micronuclei at the 2-cell stage, 3-cell stage, or 4-cell stage in order to avoid transfer of an embryo with mitotic chromosomal errors.

In another embodiment, the method comprises (a) culturing one or more embryos under conditions suitable for embryonic development; (b) observing the embryo during development from the 1-cell stage to the 4-cell stage optically, wherein observations of the embryo are made periodically at time intervals of no more than 30 minutes; and (c) selecting an embryo for transfer to the female subject, wherein the embryo has no detectable formation of embryonic micronuclei at the 1-cell stage in order to avoid transfer of an embryo with meiotic chromosomal errors.

In the practice of the invention, embryos can be monitored during development optically by any suitable method. For example, images of embryos can be obtained using a microscope, such as a confocal microscope, a light microscope, a fluorescence microscope, a digital microscope, or other high magnification imaging system. Any optical method may be used, such as bright field, dark field, phase contrast, Hoffman modulation contrast, fluorescence, or differential interference contrast. In certain embodiments, a digital camera may be used to capture images of the embryo. The camera may be coupled to a computer for receiving and processing digital data from the digital camera. The image of the embryo may be a still photo or a video in any format (e.g., bitmap, Graphics Interchange Format, JPEG file interchange format, TIFF, or mpeg). Alternatively, the image of the embryo may be captured by an analog camera and converted into an electronic form.

The embryo should be observed during development at time intervals, preferably between 1 to 30 minutes, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or any time interval within this range. In certain embodiments, the time interval between images is varied depending on the amount of cellular activity. For example, during active periods, images could be taken as often as every few seconds or every minute, while during inactive periods, images can be taken, for example, every 10 or 15 minutes or longer.

The methods of the invention may be used alone or in combination with any other method of embryo assessment, including, but not limited to measuring one or more cellular parameters, measuring gene expression levels of one or more genes in the embryo, quantitating blastomere fragmentation, detecting blastomere asymmetry, or evaluating the morphology of the embryo in culture at days 2, 3, 4, 5, or 6, or any combination thereof.

In certain embodiments, fragmentation dynamics are measured in combination with one or more cell cycle parameters including, but not limited to the time between the first and second mitosis, the time between the second and third mitosis, the duration of the first cytokinesis, the time interval between cytokinesis 1 and cytokinesis 2, and the time interval between cytokinesis 2 and cytokinesis 3. Poor developmental potential of an embryo is indicated by a cellular measurement that falls outside the normal range for one or more of the cell cycle parameters.

In other embodiments, fragmentation dynamics are measured in combination with expression levels of one or more genes in the human embryo including, but not limited to Cofillin, DIAPH1, ECT2, MYLC2/MYL5, DGCR8, Dicer/DICER1, TARBP2, CPEB1, Symplekin/SYMPK, YBX2, ZAR1, CTNNB1, DNMT3B, TERT, YY1, IFGR2/IFNGR2, BTF3, and NELF. The expression levels of one or more genes of the human embryo may be compared to the expression levels of one or more genes of a reference embryo (i.e., normal euploid embryo). Decreased expression of one or more genes selected from the group consisting of Cofillin, DIAPH1, ECT2, MYLC2/MYL5, DGCR8, Dicer/DICER1, TARBP2, CPEB1, Symplekin/SYMPK, YBX2, ZAR1, CTNNB1, DNMT3B, TERT, YY1, IFGR2/IFNGR2, BTF3 and NELF in an embryo relative to a reference embryo is indicative of poor developmental potential.

In another embodiment, fragmentation dynamics are measured in combination with the amount of fragmentation of the embryo. A high level of fragmentation greater than about 25% fragmentation by volume of cytoplasm is indicative of poor developmental potential.

In another embodiment, fragmentation dynamics are measured in combination with evaluation of blastomere shape or embryo morphology. Detection of blastomere asymmetry or abnormal morphology of the embryo in culture at days 2, 3, 4, 5, or 6, or any combination thereof, is also indicative of poor developmental potential.

In yet another embodiment, fragmentation dynamics are measured in combination with growth of the embryo to the blastocyst stage (Day 5). Embryos that have exhibited dynamic fragmentation during formation of the first four cells would preferentially not be transferred for reproductive purposes.

Any one or more of these methods can be used in any combination with monitoring fragmentation dynamics to aid in assessment of the embryo.

These and other embodiments of the subject invention will readily occur to those of skill in the art in view of the disclosure herein.

### BRIEF DESCRIPTION OF THE FIGURES

FIGS. 1A-1C show that euploid and aneuploid embryos can be distinguished using dynamic parameter analysis. FIG. 1A shows A-CGH profiles of individual blastomeres showing the copy number of each chromosome in euploid, trisomy 21, monosomy 22, high mosaic and low mosaic embryos plotted in the 2-D and 3-D graphs. Copy number is based on the log₂ ratio of the average signal intensity of the test to reference DNA for each chromosome. Low mosaic embryos exhibited losses or gains in four chromosomes or less, while more than four chromosomes were affected in high mosaic embryos. FIG. 1B shows a graphic representation of the incidence of aneuploidy observed in each chromosome for all human embryos used in the study and the surprisingly high frequency at which each chromosome is affected. FIG. 1C shows a three-dimensional (3-D) plot displaying the correlation between the timing measurements of three parameters, the duration of the first cytokinesis, the interval between the first and second mitosis and the interval between the second and third mitosis and the underlying chromosomal composition of each imaged embryo. Embryos were categorized as euploid (circles), triploid (diamonds), low mosaic (squares), high mosaic (downward pointing triangles), monosomy 22 (black asterisks), monosomy other (stars), trisomy 21 (upward pointing triangles) and trisomy other (black plus signs) based on their A-CGH results. Note that all of the euploid embryos clustered together in a similar region as non-arrested or developmentally normal embryos in a previous report (Wong et al. (2010) Nature Biotechnol. 28:1115-1121), while aneuploid embryos either overlapped with euploid embryos or accumulated at or close to zero for the second cell cycle parameter; N=45.

FIGS. 2A-2D show the association between embryonic aneuploidy, cell cycle parameters and cellular fragmentation. FIG. 2A shows the last frame of a time-lapse imaging sequence taken from an embryo with (1-indicated by white arrow) and without (2) fragmentation corresponding to the chromosomal composition outlined in Table 2a and 1b, respectively. FIG. 2B shows euploid embryos with (squares) and without (circles) fragmentation, aneuploid embryos with (triangles) and without (diamonds) fragmentation and triploid embryos with (black asterisks) and without (gray stars) fragmentation were graphed on a three-dimensional (3-D) plot. While fragmentation was detected in only one euploid embryo, the majority of both aneuploid and triploid embryos exhibited fragmentation; N=45. FIG. 2C shows that there is substantial overlap between embryos predicted to form blastocysts that do or do not exhibit fragmentation as illustrated in a 3-D plot of blastocyst prediction with (squares) and without (circles) fragments (frags) and no blastocyst prediction with (triangles) and without (diamonds) fragmentation. FIG. 2D shows a 3-D plot demonstrating that in contrast to triploid embryos with fragmentation (aqua diamonds) and those with meiotic errors and fragmentation (triangles), with the exception of one embryo with a meiotic error, only embryos with mitotic errors and fragmentation (plus signs) cluster near euploid embryos (circles); N=32.

FIGS. 3A and 3B show evidence for the sequestering of chromosomes within cellular fragments. FIG. 3A shows a three-dimensional (3-D) plot showing the relationship between correct chromosome copy number defined as 2 copies of each chromosome per blastomere minus fragmentation (frags; circles) or plus fragmentation (diamonds) and incorrect chromosome copy number minus fragmentation (squares) or plus fragmentation (triangles). Note that fragmentation may be used to identify embryos with abnormal chromosome number(s) that exhibit normal parameter timing; N=23. FIG. 3B shows fluorescent in situ hybridization (FISH) analysis of a single blastomere shown by the dashed box from a cleavage-stage embryo exhibiting cellular fragmentation and visualized by Differential Interference Contrast (DIC; 1) and confocal microscopy (2-3). Two FISH signals for chromosome 16 were detected in the primary nucleus of the blastomere (2; indicated by white solid arrow) stained with 4′,6-diamidino-2-phenylindole (DAPI) in the merged image (3), but also one chromosome 16 signal was observed outside the primary nucleus of the blastomere (shown by white dashed arrow). FIG. 3C shows a similar FISH analysis of an individual blastomere indicated by the dashed box from an embryo without fragmentation (1) showing 1-2 copies of chromosome 21 (2) in a small nuclear structure distinct from the primary nucleus of the blastomere (3).

FIGS. 4A-4E show detection and developmental consequences of embryonic micronuclei. FIG. 4A shows that LAMIN-B1 and CENP-A expression in DAPI-stained cleavage-stage human embryos by confocal microscopy reveals chromosome-containing micronuclei in the blastomeres of human embryos (shown by white arrows), but not in mouse embryos also stained with Mitotracker Red (FIG. 4B). FIG. 4C shows the detection of embryonic micronuclei by LAMIN-B1 immunostaining in human embryos with abnormal cell cycle parameters, but not in those with normal parameter timing (FIG. 4D). FIG. 4E shows a three-dimensional (3-D) plot showing the effects of micronuclei on the cell cycle parameters and embryo developmental potential. Note that embryos without micronuclei (circles) tightly cluster in a region similar to embryos with normal CGH profiles, whereas those embryos with micronuclei (triangles) exhibit more diverse parameter clustering when graphed; N=8.

FIGS. 5A-5D show that fragmentation timing indicates an embryo response to chromosomal abnormalities. FIG. 5A shows individual frames indicated by numbers taken from a time-lapse imaging sequence of an embryo with fragmentation demonstrating the fusion of a cellular fragment with an embryonic blastomere, which helps explain the complexity and incongruence of aneuploidy detected in human embryonic blastomeres. FIG. 5B shows numbered imaging frames showing the incidence of cellular fragmentation following completion of the first cytokinesis. FIG. 5C shows a proposed model for the origin of human embryonic aneuploidy based on fragmentation timing, fragment resorption and underlying chromosomal abnormalities. Human embryos with meiotic errors (monosomies and trisomies) and those that appear to be triploid typically exhibit fragmentation at the 1-cell stage, while fragmentation is most often detected at the 2-cell stage in embryos with mitotic errors. We also demonstrate that missing chromosome(s) are contained within fragments (FIG. 15A) termed embryonic micronuclei and for those embryos with mitotic errors, propose that the embryo likely divided before these chromosomes properly aligned on the mitotic spindle. The correlation between the timing of fragmentation and the type of embryonic aneuploidy suggests that the embryo may respond to chromosomal abnormalities and undergoes fragmentation as a survival mechanism. As development proceeds, these fragments either remain or are reabsorbed by the blastomere from which they originated or a neighboring blastomere to generate the complex human aneuploidies observed. FIG. 5D shows a 3-Dimensional (3-D) plot showing the relationship between the timing of fragmentation and the cell cycle imaging parameters. Note that fragmentation, which is observed at the 1-cell stage or later in development at the 3-4 cell stage has more detrimental effects on the imaging parameters than fragmentation that occurs at the 2-cell stage; N=32.

FIG. 6 shows a summary model of human embryonic aneuploidy based on observations of correlated imaging and chromosomal analysis. Embryonic development was monitored by time-lapse imaging from the 1- to the 4-cell stage followed by assessment of chromosomal composition of each blastomere in the imaged embryos. We observed refinement of diagnostic non-invasive cell cycle parameters, determined the correlation with meiotic (monosomies and trisomies) and mitotic (high and low mosaic) errors and demonstrated an association between the cell cycle parameters and embryo morphology (fragmentation and blastomere asymmetry). We also suggest clinical value of parameter analysis with and without automated fragmentation assessment.

FIGS. 7A-7C show an experimental design of embryonic aneuploidy experiments using time-lapse image and chromosome analysis. FIG. 7A shows how the development of 75 embryos at the 1-cell (2 pronuclear; 2PN) stage was tracked in five separate experiments. For each experimental set, human zygotes were thawed on day 1 and cultured together in alphabetically and numerically labeled microwell containing petri dishes. Time-lapse imaging, using a custom-built microscope with dark-field illumination placed in a standard incubator, was performed until day 2 (approximately 30 hours). Once the majority had reached the 4-cell stage, the embryos were removed and dissembled into single blastomeres using the microwell labels for embryo identification. The chromosomal complement of each blastomere was evaluated by Array-Comparative Genomic Hybridization (A-CGH), the results of which were correlated with embryo imaging behavior by manually measuring dynamic imaging parameters between the first (FIG. 7B) and last (FIG. 7C) frame of an image sequence compiled into a time-lapse movie with well identification labels and time stamps.

FIGS. 8A-8D show differential clustering of euploid embryos and embryos with meiotic and mitotic errors. FIG. 8A shows a 3-dimensional (3-D) parameter plot demonstrating the separation of euploid embryos (circles) from triploid embryos (diamonds) and embryos with meiotic errors (triangles) and partial overlap of embryos with mitotic errors (plus signs); N=45. FIG. 8B shows the 2-D parameter plot of euploid, triploid and meiotic/mitotic error embryos illustrated in FIG. 8A. FIG. 8C shows a similar plot of euploid, triploid, monosomy 22 (black asterisks), monosomy other (stars), trisomy 21 (triangles) and trisomy other (black plus signs) embryos showing the distinction between euploid embryos and those with different meiotic errors; N=20. FIG. 8D shows that further examination of the relationship between euploid, triploid, high (triangles) or low (squares) mosaic mitotic embryos by 3-D parameter plotting reveals that the overlap between euploid and mitotic error embryos are predominantly those with low mosaicism; N=36.

FIGS. 9A and 9B show the detection of chromosome copy number in triploid human embryos by FISH. FIG. 9A shows the last frame in an imaging sequence of an embryo with fragmentation (indicated by the white arrow) that appeared to be triploid based on imaging parameter assessment. FIG. 9B shows fluorescent in situ hybridization (FISH) analysis of a blastomere from the embryo pictured in FIG. 9A showing three copies of chromosome 18 (1; indicated by white arrows) to confirm the embryo as triploid and female since no signals for the Y-chromosome (2) were detected.

FIGS. 10A-10C show the parameter timing of aneuploid embryos with additional sub-chromosomal errors. FIG. 10A shows A-CGH profiles of an aneuploid embryo with a balanced partial loss and gain of chromosome 1q between two blastomeres that did not exhibit fragmentation. FIG. 10B shows an A-CGH profile of an individual blastomere from an embryo with fragmentation showing a partial loss of chromosome 1q, partial gain of chromosome 9q, partial loss of chromosome 10q and a partial loss chromosome 16q. FIG. 10C show a 3-dimensional (3-D) parameter plot showing the cell cycle parameter timing of low mitotic mosaic (circles), high mitotic mosaic (plus signs) and triploid embryos (squares) that also exhibited either balanced or unbalanced chromosomal losses and/or gains depicted in Table 3; N=10.

FIG. 11 shows the assessment of risk of embryonic euploidy versus aneuploidy with a risk tree showing the probability of embryonic euploidy (circles) if no morphological or parameter screening (diamond), high fragmentation screening (octagon), low fragmentation screening (downward pointing pentagon), cell cycle parameters that predict blastocyst formation (upward pointing pentagon) and cell cycle parameters that predict normal CGH (triangles) were used to assess developmental competence. The probability of embryonic euploidy in the absence of morphological or parameter assessment, or essentially the chances of obtaining an aneuploid embryo at random, is 17.8% (Table 4), which is in agreement with Vanneste et al. (Nat. Med. (2009)15:577-583). By triaging embryos via high and low fragmentation as a screening tool, this percentage increased to 26.7% and 60%, respectively. However, only 65% of the embryos assessed via fragmentation screening alone exhibited timing values that would be predictive of blastocyst formation (white hexagon), indicating that fragmentation alone is not sufficient for the prediction of embryo viability, especially when it is assessed at a single time point (FIG. 3C). Using our previously defined cell cycle parameter timing windows to predict blastocyst formation (Wong et al. (2010) Nat. Biotechnol. 28:1115-1121), the probability of embryonic euploidy in the present study is 40% and if the refined parameter timing windows that predict normal chromosome complement as determined in this study were applied, this percentage increased to 63.6%. Finally, when fragmentation assessment was implemented in conjunction with cell cycle parameter analysis, the probability of embryonic euploidy increased to 87.5% (Table 4).

FIGS. 12A-12C show a graphic representation of the probability of embryonic euploidy versus aneuploidy with single or multiple embryo transfer. Graphs representing the calculated results in Table 5 are used to illustrate the probability of embryonic euploidy versus aneuploidy when (FIG. 12A) 1 embryo (FIG. 12B) 2 embryo or (FIG. 12C) 3 embryos were selected for patient transfer using only high fragmentation assessment since according to current clinical practice, embryos with greater than 25% fragmentation by cytoplasmic volume on day 3 are graded as poor quality and associated with significantly decreased live birth rates (Zhang et al. (2009) PLoS One 4, e7844; Galan et al. (2010) PLoS ONE 5, e13615). Analogous to the findings observed with single embryos, fragmentation screening alone had minimal effect on the probability of embryonic aneuploidy in comparison to the cell cycle parameters or the parameters in combination with both high and low fragmentation assessment when a 2- or 3-embryo transfer scenario was used.

FIGS. 13A-13C shows the assistance of additional fragmentation criteria in the assessment of developmental competence. FIG. 13A shows a 3-dimensional (3-D) plot showing the relationship between euploid (circles), triploid (diamonds), high (triangles) or low (squares) mosaic mitotic embryos and fragmentation. Of the embryos with mitotic errors that exhibited fragmentation, 5 out of 9 and 1 out of 11 embryos that clustered in a region similar to that of euploid embryos had underlying low mosaic and high mosaic mitotic errors, respectively; N=23. FIG. 13B shows that an examination of high (circles) and low (diamonds) fragmentation by 3-D plotting reveals that all highly fragmented embryos exhibit abnormal parameter timing. A high degree of fragmentation was measured as more than 25% fragmentation by volume of cytoplasm and a low degree of fragmentation was measured as less than 25% fragmentation by volume of cytoplasm. FIG. 13C shows a 3-D parameter plot of embryos with symmetrical blastomeres plus (circles) or minus (plus signs) fragmentation and those embryos exhibiting blastomere asymmetry with (squares) and without (triangles) fragments. Note that asymmetrical embryos are more likely to be fragmented and have abnormal parameter timing; N=45.

FIGS. 14A-14C show further proof of nuclear DNA within cellular fragments of cleaving human embryos. FIG. 14A shows a single confocal image frame of a zona pellucida-free cleavage-stage embryo with cellular fragmentation (indicated by black arrows) visualized by Differential Interference Contrast (DIC) showing positive DAPI signals (indicated by white solid arrow) in a fragment (indicated by a solid black arrow) adjacent to a DAPI-negative fragment (indicated by a dashed black arrow). FIGS. 14B and 14C show 3-D modeling of an embryo with fragments imaged by DIC (left) and Z-stack confocal microscopy (right) exhibiting one and two DAPI positive fragments, respectively.

FIGS. 15A-15C show additional evidence of embryonic micronuclei in the blastomeres of only human embryos. FIG. 15A shows confocal imaging of LAMIN-B1 and CENP-A expression in DAPI-stained cleavage-stage human embryos showing the appearance of embryonic micronuclei and/or chromosome-containing fragments. FIG. 15B shows a similar imaging analysis for LAMIN-B1 expression in another DAPI-stained embryo also visualized by DIC. Note the presence of micronuclei enapasulated by LAMIN-B1 expression in the blastomeres of human embryos, but not (FIG. 15C) mouse embryos also stained with Mitotracker Red.

FIGS. 16A and 16B show the detection of micronuclei in embryos with normal and abnormal parameter timing. FIG. 16A shows a confocal imaging of LAMIN-B1 in additional DAPI-stained and DIC visualized human embryos with (FIG. 16A) abnormal and (FIG. 16B) normal cell cycle parameters plotted in FIG. 4E.

FIGS. 17A and 17B show additional evidence of embryonic micronuclei in the blastomeres of human embryos. Confocal imaging of LAMIN-B1 and CENP-A expression in DAPI-stained cleavage-stage human embryos without DIC image analysis shows the appearance of (FIG. 17A) embryonic micronuclei and/or (FIG. 17B) chromosome-containing fragments indicated by white arrows.

FIGS. 18A and 18B show non-invasive detection of embryonic micronuclei by time-lapse imaging. FIG. 18A shows a time-lapse image sequence taken by brightfield image analysis of a human embryo showing the appearance of embryonic micronuclei/fragments (FIG. 18B) distinct from the primary nuclei observed in the brightfield imaging sequence of another human embryo without fragmentation.

FIG. 19 shows embryonic micronuclei detection in human Germinal Vesicle (GV) oocytes. DIC and confocal imaging of LAMIN-B1 in a DAPI-stained human Germinal Vesicle (GV) oocyte shows that the GV oocyte failed to properly mature. Note the LAMIN-B1 encapsulated micronucleus protruding from the primary nucleus of the oocyte (indicated by white arrow), which suggests embryonic micronuclei can appear early in development even before oocyte maturation or fertilization and may have detrimental affects on meiotic progression and subsequent mitotic divisions following fertilization.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., Handbook of Experimental Immunology, Vols. I-IV (D. M. Weir and C.C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a blastomere” includes a mixture of two or more blastomeres, and the like.

The term “about”, particularly in reference to a given quantity, is meant to encompass deviations of plus or minus five percent.

The terms “developmental potential” and “developmental competence” are used herein to refer to the ability or capacity of a healthy embryo or pluripotent cell to grow or develop.

The term “embryo” is used herein to refer both to the zygote that is formed when two haploid gametic cells, e.g. an unfertilized secondary oocyte and a sperm cell, unite to form a diploid totipotent cell, e.g. a fertilized ovum, and to the embryo that results from the immediately subsequent cell divisions, i.e. embryonic cleavage, up through the morula, i.e. 16-cell stage and the blastocyst stage (with differentiated trophoectoderm and inner cell mass).

The term “embryonic micronuclei” is used herein to refer to embryonic structures, containing one or more encapsulated chromosomes, distinct from the primary nuclei or mitochondrial DNA in blastomeres.

The term “oocyte” is used herein to refer to an unfertilized female germ cell, or gamete. Oocytes of the subject application may be primary oocytes, in which case they are positioned to go through or are going through meiosis I, or secondary oocytes, in which case they are positioned to go through or are going through meiosis II.

By “meiosis” it is meant the cell cycle events that result in the production of gametes. In the first meiotic cell cycle, or meiosis I, a cell's chromosomes are duplicated and partitioned into two daughter cells. These daughter cells then divide in a second meiotic cell cycle, or meiosis II, that is not accompanied by DNA synthesis, resulting in gametes with a haploid number of chromosomes.

By the “germinal vesicle” stage it is meant the stage of a primary oocyte's maturation that correlates with prophase I of the meiosis I cell cycle, i.e. prior to the first division of the nuclear material. Oocytes in this stage are also called “germinal vesicle oocytes”, for the characteristically large nucleus, called a germinal vesicle. In a normal human oocyte cultured in vitro, germinal vesicle occurs about 6-24 hours after the start of maturation.

By the “metaphase I” stage it is meant the stage of a primary ooctye's maturation that correlates with metaphase I of the meiosis I cell cycle. In comparison to germinal vesicle oocytes, metaphase I oocytes do not have a large, clearly defined nucleus. In a normal human oocyte cultured in vitro, metaphase I occurs about 12-36 hours after the start of maturation.

By the “metaphase II” stage it is meant the stage of a secondary ooctye's maturation that correlates with metaphase II of the meiosis II cell cycle. Metaphase II is distinguishable by the extrusion of the first polar body. In a normal human oocyte cultured in vitro, metaphase II occurs about 24-48 hours after the start of maturation.

By a “mitotic cell cycle”, it is meant the events in a cell that result in the duplication of a cell's chromosomes and the division of those chromosomes and a cell's cytoplasmic matter into two daughter cells. The mitotic cell cycle is divided into two phases: interphase and mitosis. In interphase, the cell grows and replicates its DNA. In mitosis, the cell initiates and completes cell division, first partitioning its nuclear material, and then dividing its cytoplasmic material and its partitioned nuclear material (cytokinesis) into two separate cells.

By a “first mitotic cell cycle” or “cell cycle 1” it is meant the time interval from fertilization to the completion of the first cytokinesis event, i.e. the division of the fertilized oocyte into two daughter cells. In instances in which oocytes are fertilized in vitro, the time interval between the injection of human chorionic gonadotropin (HCG) (usually administered prior to oocyte retrieval) to the completion of the first cytokinesis event may be used as a surrogate time interval.

By a “second mitotic cell cycle” or “cell cycle 2” it is meant the second cell cycle event observed in an embryo, the time interval between the production of daughter cells from a fertilized oocyte by mitosis and the production of a first set of granddaughter cells from one of those daughter cells (the “leading daughter cell”, or daughter cell A) by mitosis. Upon completion of cell cycle 2, the embryo consists of 3 cells. In other words, cell cycle 2 can be visually identified as the time between the embryo containing 2-cells and the embryo containing 3-cells.

By a “third mitotic cell cycle” or “cell cycle 3” it is meant the third cell cycle event observed in an embryo, typically the time interval from the production of daughter cells from a fertilized oocyte by mitosis and the production of a second set of granddaughter cells from the second daughter cell (the “lagging daughter cell” or daughter cell B) by mitosis. Upon completion of cell cycle 3, the embryo consists of 4 cells. In other words, cell cycle 3 can be visually identified as the time between the embryo containing 3-cells and the embryo containing 4-cells.

By “first cleavage event”, it is meant the first division, i.e. the division of the oocyte into two daughter cells, i.e. cell cycle 1. Upon completion of the first cleavage event, the embryo consists of 2 cells.

By “second cleavage event”, it is meant the second set of divisions, i.e. the division of leading daughter cell into two granddaughter cells and the division of the lagging daughter cell into two granddaughter cells. In other words, the second cleavage event consists of both cell cycle 2 and cell cycle 3. Upon completion of second cleavage, the embryo consists of 4 cells.

By “third cleavage event”, it is meant the third set of divisions, i.e. the divisions of all of the granddaughter cells. Upon completion of the third cleavage event, the embryo typically consists of 8 cells.

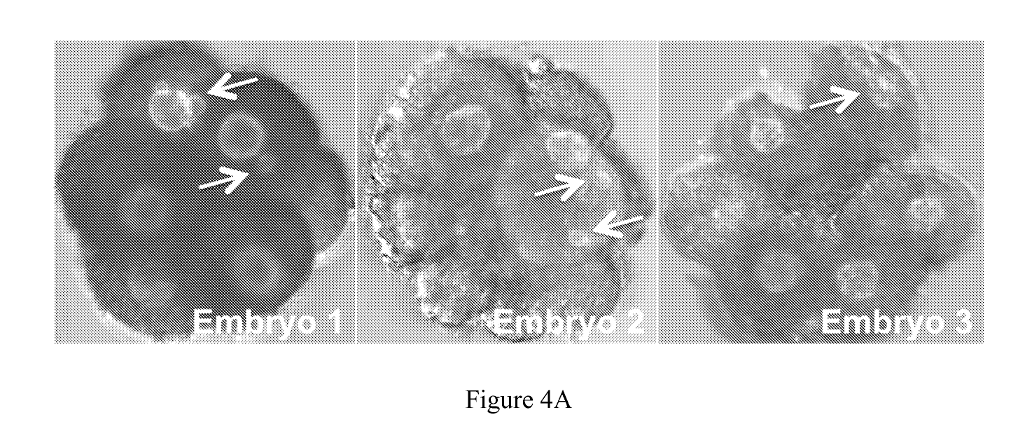

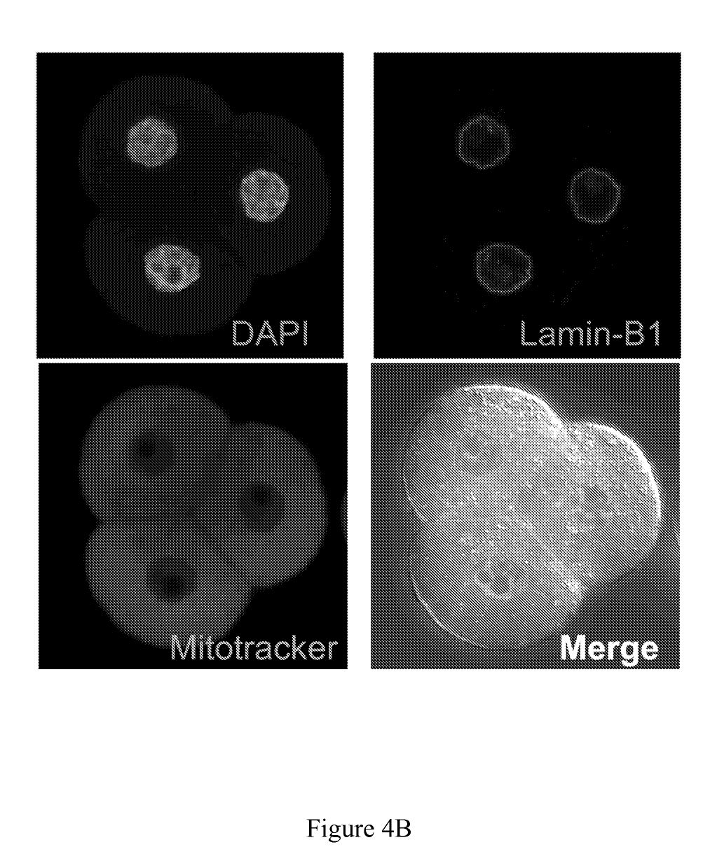

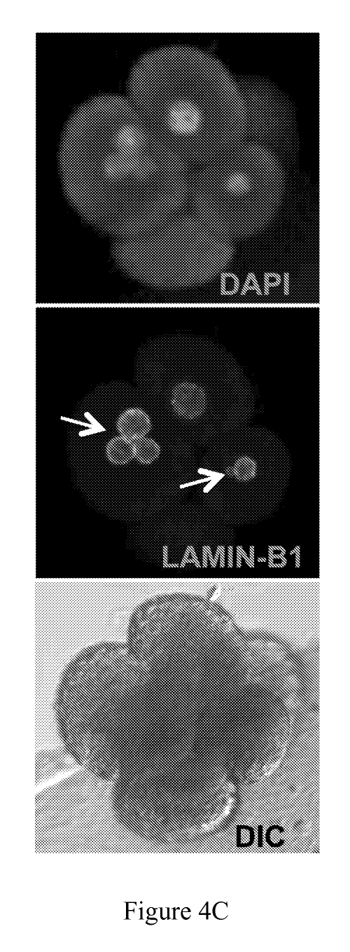

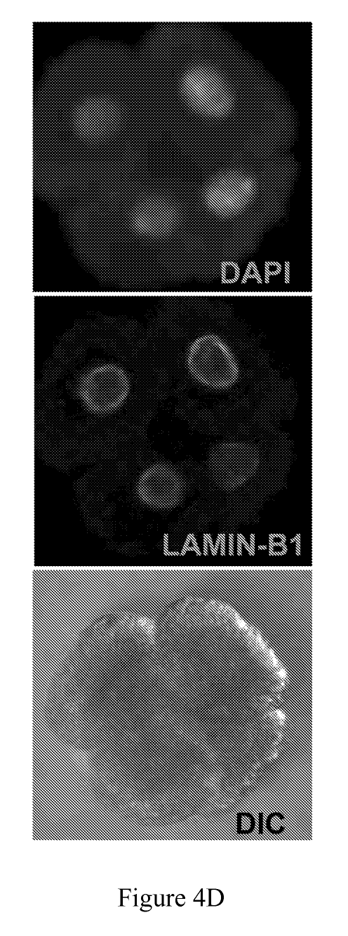

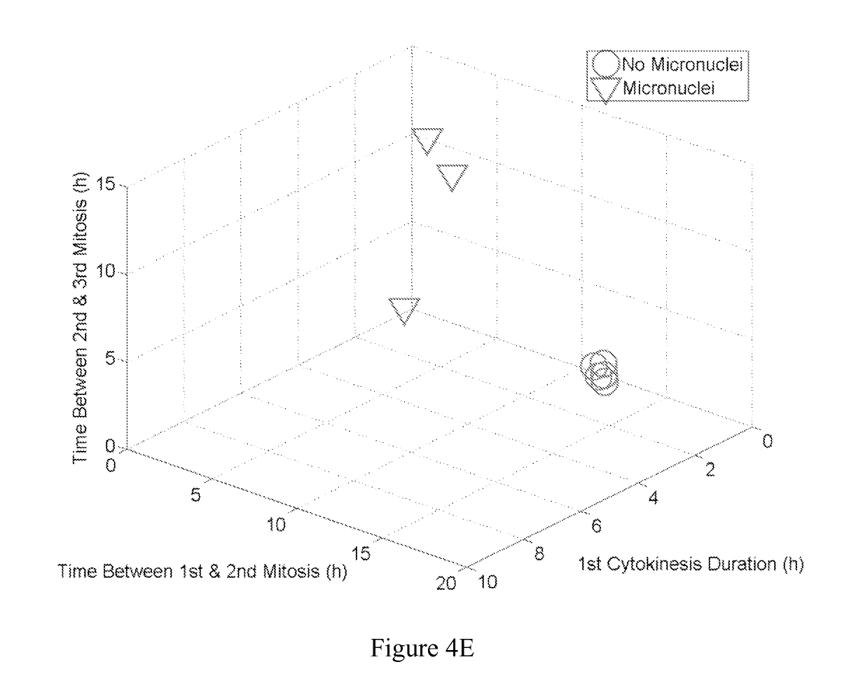

By “cytokinesis” or “cell division” it is meant that phase of mitosis in which a cell undergoes cell division. In other words, it is the stage of mitosis in which a cell's partitioned nuclear material and its cytoplasmic material are divided to produce two daughter cells. The period of cytokinesis is identifiable as the period, or window, of time between when a constriction of the cell membrane (a “cleavage furrow”) is first observed and the resolution of that constriction event, i.e. the generation of two daughter cells. The initiation of the cleavage furrow may be visually identified as the point in which the curvature of the cell membrane changes from convex (rounded outward) to concave (curved inward with a dent or indentation). This is illustrated in FIG. 4 top panel by white arrows pointing at 2 cleavage furrows. The onset of cell elongation may also be used to mark the onset of cytokinesis, in which case the period of cytokinesis is defined as the period of time between the onset of cell elongation and the resolution of the cell division.

By “first cytokinesis” or “cytokinesis 1” it is meant the first cell division event after fertilization, i.e. the division of a fertilized oocyte to produce two daughter cells. First cytokinesis usually occurs about one day after fertilization.

By “second cytokinesis” or “cytokinesis 2”, it is meant the second cell division event observed in an embryo, i.e. the division of a daughter cell of the fertilized oocyte (the “leading daughter cell”, or daughter A) into a first set of two granddaughters.

By “third cytokinesis” or “cytokinesis 3”, it is meant the third cell division event observed in an embryo, i.e. the division of the other daughter of the fertilized oocyte (the “lagging daughter cell”, or daughter B) into a second set of two granddaughters.

The term “fiduciary marker” or “fiducial marker,” is an object used in the field of view of an imaging system which appears in the image produced, for use as a point of reference or a measure. It may be either something placed into or on the imaging subject, or a mark or set of marks in the reticle of an optical instrument.

The term “micro-well” refers to a container that is sized on a cellular scale, preferably to provide for accommodating a single eukaryotic cell.

By “aneuploidy” is meant a type of chromosomal abnormality characterized by an abnormal number of chromosomes. Aneuploid embryos can have one or more missing chromosomes and/or one or more extra chromosomes. The aneuploidy can be a result of a “mitotic error” or a “meiotic error.” An “aneuploid embryo” is an embryo which contains an aneuploidy.

By “euploid” is meant an embryo that is characterized as being chromosomally normal. Euploid, or normal embryos have the proper number of chromosome pairs. A euploid human embryo for example has 23 pairs of chromosomes for a total of 46 chromosomes.

By “mitotic error” is meant a type of aneuploidy caused by an error of chromosome separation during mitosis.

By “meiotic error” is meant a type of aneuploidy caused by an error of chromosome separation during meiosis.

By “trisomy” is meant a type of aneuploidy in which there are three copies of a particular chromosome instead of two. A “trisomy embryo” is an embryo in which the chromosome count is abnormal and contains three copies of one or more chromosomes instead of two copies.

By “monosomy” is meant a type of aneuploidy in which there is one copy of a particular chromosome instead of two. A “monosomy embryo” is an embryo in which the chromosome count is abnormal and contains one copy of one or more chromosomes instead of two copies.

By “normal chromosome count” is meant two copies of each of the 23 chromosomes for humans.

By “mosaic” or “mosaicism” is meant a population of cells with different chromosomal content. A “mosaic embryo” is an embryo which contains populations of cells with different chromosomal content.

By “low mosaic” is meant a population of cells with different chromosomal content wherein four or less chromosomes that arose from a mitotic error are affected.

By “high mosaic” is meant a population of cells with different chromosomal content wherein more than four chromosomes that arose from a mitotic error are affected.

By “high levels of fragmentation” is meant more than about 25% fragmentation by volume of cytoplasm.

By “low levels of fragmentation” is meant about 25% or less fragmentation by volume of cytoplasm.

By “fragmentation” is meant portions of membrane bound cytoplasm that may or may not contain nuclear DNA.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that monitoring the dynamics of embryonic cellular fragmentation can be used to distinguish euploid and aneuploid embryos. In particular, the inventors have discovered that the timing of cellular fragmentation is diagnostic of blastomeric chromosomal composition (see Example 1). The inventors have further shown that monitoring embryos from the 1-cell stage to the 4-cell stage by non-invasive time lapse imaging may aid in the assessment of embryonic developmental potential and allow female subjects undergoing in vitro fertilization to avoid transfer of non-viable embryos (see Example 1). In order to further an understanding of the invention, a more detailed discussion is provided below regarding methods of evaluating embryonic developmental potential by monitoring embryonic fragmentation dynamics.

A. Assessment of Blastomere Fragmentation Dynamics

In one aspect, the invention includes methods for monitoring blastomere fragmentation dynamics during development of an embryo. The timing of the appearance of embryonic micronuclei or cellular fragmentation during development of an embryo is diagnostic of blastomeric chromosomal composition. Therefore, monitoring fragmentation dynamics is useful for detecting aneuploidy and evaluating developmental potential of an embryo. In one embodiment, the invention includes a method for detecting aneuploidy in an embryo, the method comprising: (i) observing the embryo optically during development from the 1-cell stage to the 4-cell stage; and (ii) detecting embryonic micronuclei or cellular fragmentation if present, wherein the presence of embryonic micronuclei or cellular fragmentation indicates the embryo is aneuploid.

More specifically, the timing of the appearance of embryonic micronuclei or cellular fragmentation during development of the embryo can be correlated with the type of aneuploidy and the severity of chromosomal errors (see Example 1). Embryos with meiotic errors and triploid embryos typically have embryonic micronuclei or exhibit fragmentation beginning at the 1-cell stage prior to the first cytokinesis. In contrast, embryos with mitotic errors typically have embryonic micronuclei or exhibit fragmentation following the division of 1-cell to 2-cells or later in development at the 3-cell or 4-cell stage. Accordingly, the timing of the appearance of embryonic micronuclei or cellular fragmentation can be correlated with the developmental potential of the embryo.

Thus, in another aspect, the invention includes a method for evaluating the developmental potential of an embryo, the method comprising: (a) observing the embryo during development optically and (b) detecting embryonic micronuclei or cellular fragmentation if present, wherein the timing of the appearance of the embryonic micronuclei or cellular fragmentation is correlated with the developmental potential of the embryo. In one embodiment, the appearance of embryonic micronuclei or cellular fragmentation at the 1-cell stage is correlated with the likelihood of embryonic lethality arising from meiotic chromosomal errors. In another embodiment, the appearance of embryonic micronuclei or cellular fragmentation at the 2-cell, 3-cell, or 4-cell stage is correlated with the likelihood of embryonic lethality arising from mitotic chromosomal errors.

In another aspect, the invention includes a method for selecting a human embryo with favorable developmental potential for transfer to a female subject. In one embodiment, the method comprises (a) culturing an embryo under conditions suitable for embryonic development, (b) observing the embryo during development from the 1-cell stage to the 4-cell stage optically, wherein observations of the embryo are made periodically at time intervals of no more than 30 minutes, and (c) selecting an embryo for transfer to the female subject, wherein the embryo has no detectable formation of embryonic micronuclei or cellular fragmentation during development from the 1-cell stage to the 4-cell stage.

In another embodiment, the method comprises (a) culturing one or more embryos under conditions suitable for embryonic development; (b) observing the embryo during development from the 1-cell stage to the 4-cell stage optically, wherein observations of the embryo are made periodically at time intervals of no more than 30 minutes; and (c) selecting an embryo for transfer to the female subject, wherein the embryo has no detectable cellular fragmentation at the 2-cell stage, 3-cell stage, or 4-cell stage in order to avoid transfer of an embryo with mitotic chromosomal errors.

In another embodiment, the method comprises (a) culturing one or more embryos under conditions suitable for embryonic development; (b) observing the embryo during development from the 1-cell stage to the 4-cell stage optically, wherein observations of the embryo are made periodically at time intervals of no more than 30 minutes; and (c) selecting an embryo for transfer to the female subject, wherein the embryo has no detectable cellular fragmentation at the 1-cell stage in order to avoid transfer of an embryo with meiotic chromosomal errors.

In another embodiment, the method comprises (a) culturing one or more embryos under conditions suitable for embryonic development; (b) observing the embryo during development from the 1-cell stage to the 4-cell stage optically, wherein observations of the embryo are made periodically at time intervals of no more than 30 minutes; and (c) selecting an embryo for transfer to the female subject, wherein the embryo has no detectable formation of embryonic micronuclei at the 2-cell stage, 3-cell stage, or 4-cell stage in order to avoid transfer of an embryo with mitotic chromosomal errors.

In another embodiment, the method comprises (a) culturing one or more embryos under conditions suitable for embryonic development; (b) observing the embryo during development from the 1-cell stage to the 4-cell stage optically, wherein observations of the embryo are made periodically at time intervals of no more than 30 minutes; and (c) selecting an embryo for transfer to the female subject, wherein the embryo has no detectable formation of embryonic micronuclei at the 1-cell stage in order to avoid transfer of an embryo with meiotic chromosomal errors.

The methods of the invention may be used alone or in combination with any other method of embryo assessment, including, but not limited to measuring one or more cell cycle parameters, measuring gene expression levels of one or more genes in the embryo, quantitating blastomere fragmentation, detecting blastomere asymmetry, or evaluating the morphology of the embryo in culture at days 2, 3, 4, 5, or 6, or any combination thereof.

Embryos may be derived from any organism, e.g. any mammalian species, e.g. human, primate, equine, bovine, porcine, canine, feline, etc. Preferable, they are derived from a human. They may be previously frozen, e.g. embryos cryopreserved at the 1-cell stage and then thawed, or frozen and thawed oocytes and stem cells. Alternatively, they may be freshly prepared, e.g., embryos that are freshly prepared from oocytes by in vitro fertilization or intracytoplasmic sperm injection techniques; oocytes that are freshly harvested and/or freshly matured through in vitro maturation techniques or that are derived from pluripotent stem cells differentiated in vitro into germ cells and matured into oocytes; stem cells freshly prepared from the dissociation and culturing of tissues by methods known in the art; and the like. They may be cultured under any convenient conditions known in the art to promote survival, growth, and/or development of the sample to be assessed, e.g. for embryos, under conditions such as those used in the art of in vitro fertilization or intracytoplasmic sperm injection; see, e.g., U.S. Pat. No. 6,610,543, U.S. Pat. No. 6,130,086, U.S. Pat. No. 5,837,543, the disclosures of which are incorporated herein by reference; for oocytes, under conditions such as those used in the art to promote oocyte maturation; see, e.g., U.S. Pat. No. 5,882,928 and U.S. Pat. No. 6,281,013, the disclosures of which are incorporated herein by reference; for stem cells under conditions such as those used in the art to promote proliferation, see, e.g. U.S. Pat. No. 6,777,233, U.S. Pat. No. 7,037,892, U.S. Pat. No. 7,029,913, U.S. Pat. No. 5,843,780, and U.S. Pat. No. 6,200,806, US Application No. 2009/0047263; US Application No. 2009/0068742, the disclosures of which are incorporated herein by reference. Often, the embryos cells are cultured in a commercially available medium such as KnockOut DMEM, DMEM-F12, or Iscoves Modified Dulbecco's Medium that has been supplemented with serum or serum substitute, amino acids, and growth factors tailored to the needs of the particular embryo being assessed.

The methods described herein may be used to guide clinical decisions, such as whether or not to transfer an in vitro fertilized embryo to a female subject and allow physicians to avoid transfer of aneuploid or non-viable embryos likely to result in miscarriage.

B. Imaging of Embryos

Embryos can be monitored during development optically by any suitable method. For example, images of embryos can be obtained using a microscope, such as a confocal microscope, a light microscope, a fluorescence microscope, a digital microscope, or other high magnification imaging system. In certain embodiments, a digital camera may be used to capture images of the embryo. The camera may be coupled to a computer for receiving and processing digital data from the digital camera. The image of the sample may be a still photo or a video in any format (e.g., bitmap, Graphics Interchange Format, JPEG file interchange format, TIFF, or mpeg). Alternatively, the image of the embryo may be captured by an analog camera and converted into an electronic form.

The embryos may be cultured in standard culture dishes or in custom culture dishes, e.g. custom culture dishes with optical quality micro-wells, as described herein. In such custom culture dishes, each micro-well holds a single embryo, and the bottom surface of each micro-well has an optical quality finish such that the entire group of embryos within a single dish can be imaged simultaneously by a single miniature microscope with sufficient resolution to follow the cell mitosis processes. The entire group of micro-wells shares the same media drop in the culture dish, and can also include an outer wall positioned around the micro-wells for stabilizing the media drop, as well as fiducial markers placed near the micro-wells. The hydrophobicity of the surface can be adjusted with plasma etching or another treatment to prevent bubbles from forming in the micro-wells when filled with media. Regardless of whether a standard culture dish or a custom culture dish is utilized, during culture, one or more developing embryos may be cultured in the same culture medium, e.g. between 1 and 25 embryos may be cultured per dish.

In certain embodiments, the embryos are monitored during development by time-lapse imaging. Images are acquired over time, and are then analyzed to determine the timing of the appearance of any embryonic micronuclei or cellular fragmentation, or optionally for measurement of one or more other cellular parameters. Time-lapse imaging may be performed with any computer-controlled microscope that is equipped for digital image storage and analysis, for example, inverted microscopes equipped with heated stages and incubation chambers, or custom built miniature microscope arrays that fit inside a conventional incubator. The array of miniature microscopes enables the concurrent culture of multiple dishes of samples in the same incubator, and is scalable to accommodate multiple channels with no limitations on the minimum time interval between successive image capture. Using multiple microscopes eliminates the need to move the sample, which improves the system accuracy and overall system reliability. The individual microscopes in the incubator can be partially or fully isolated, providing each culture dish with its own controlled environment. This allows dishes to be transferred to and from the imaging stations without disturbing the environment of the other samples.

The imaging system may employ brightfield illumination, darkfield illumination, phase contrast, Hoffman modulation contrast, differential interference contrast, or fluorescence. In some embodiments, darkfield illumination may be used to provide enhanced image contrast for subsequent feature extraction and image analysis. In addition, red or near-infrared light sources may be used to reduce phototoxicity and improve the contrast ratio between cell membranes and the inner portion of the cells.

Images that are acquired may be stored either on a continuous basis, as in live video, or on an intermittent basis, as in time-lapse photography, where a subject is repeatedly imaged in a still picture. Preferably, the time interval between images should be between 1 to 30 minutes in order to capture significant morphological events as described below, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes, or any time within this range. In an alternative embodiment, the time interval between images could be varied depending on the amount of cell activity. For example, during active periods images could be taken as often as every few seconds or every minute, while during inactive periods images could be taken every 5, 10 or 15 minutes or longer. Real-time image analysis on the captured images could be used to detect when and how to vary the time intervals. In our methods, the total amount of light received by the samples is estimated to be equivalent to approximately 24 minutes of continuous low-level light exposure for 5-days of imaging. The light intensity for a time-lapse imaging systems is significantly lower than the light intensity typically used on an assisted reproduction microscope due to the low-power of the LEDs (for example, using a 1 W LED compared to a typical 100 W Halogen bulb) and high sensitivity of the camera sensor. Thus, the total amount of light energy received by an embryo using the time-lapse imaging system is comparable to or less than the amount of energy received during routine handling at an IVF clinic. In addition, exposure time can be significantly shortened to reduce the total amount of light exposure to the embryo. For 2-days of imaging, with images captured every 5 minutes at 0.5 seconds of light exposure per image, the total amount of low-level light exposure is less than 5 minutes.

Following image acquisition, the images are extracted and analyzed to determine the degree and timing of fragmentation or appearance of embryonic micronuclei, and optionally other cellular parameters, such as the thickness of the zona pellucida, symmetry of daughter cells resulting from a cell division, time intervals between the first few mitoses, and duration of cytokinesis.

Cell parameters that may be measured by time-lapse imaging are usually morphological events. For example, in assessing embryos, time-lapse imaging may be used to measure the duration of a cytokinesis event, e.g. cytokinesis 1, cytokinesis 2, cytokinesis 3, or cytokinesis 4, where the duration of a cytokinesis event is defined as the time interval between the first observation of a cleavage furrow (the initiation of cytokinesis) and the resolution of the cleavage furrow into two daughter cells (i.e. the production of two daughter cells). Another parameter of interest is the duration of a cell cycle event, e.g. cell cycle 1, cell cycle 2, cell cycle 3, or cell cycle 4, where the duration of a cell cycle event is defined as the time interval between the production of a cell (for cell cycle 1, the fertilization of an ovum; for later cell cycles, at the resolution of cytokinesis) and the production of two daughter cells from that cell. Other cell parameters of interest that can be measured by time-lapse imaging include time intervals that are defined by these cellular events, e.g. (a) the time interval between cytokinesis 1 and cytokinesis 2, definable as any one of the interval between initiation of cytokinesis 1 and the initiation of cytokinesis 2, the interval between the resolution of cytokinesis 1 and the resolution of cytokinesis 2, the interval between the initiation of cytokinesis 1 and the resolution of cytokinesis 2; or the interval between the resolution of cytokinesis 1 and the initiation of cytokinesis 2; or (b) the time interval between cytokinesis 2 and cytokinesis 3, definable as any one of the interval between the initiation of cytokinesis 2 and the initiation of cytokinesis 3, or the interval between resolution of the cytokinesis 2 and the resolution of cytokinesis 3, or the interval between initiation of cytokinesis 2 and the resolution of cytokinesis 3, or the interval between resolution of cytokinesis 2 and the initiation of cytokinesis 3.

For the purposes of in vitro fertilization and intracytoplasmic sperm injection, it is considered advantageous that the embryo be transferred to the uterus early in development, e.g. by day 2 or day 3, i.e. up through the 8-cell stage, to reduce embryo loss due to disadvantages of culture conditions relative to the in vitro environment, and to reduce potential adverse outcomes associated with epigenetic errors that may occur during culturing (Katari et al. (2009) Hum Mol Genet. 18(20):3769-78; Sepulveda et al. (2009) Fertil Steril. 91(5):1765-70). Accordingly, it is preferable that the measurement of cellular parameters take place within 2 days of fertilization, although longer periods of analysis, e.g. about 36 hours, about 54 hours, about 60 hours, about 72 hours, about 84 hours, about 96 hours, or more, are also contemplated by the present methods.

Examples of cell parameters in a maturing oocyte that may be assessed by time-lapse imaging include, without limitation, changes in morphology of the oocyte membrane, e.g. the rate and extent of separation from the zona pellucida; changes in the morphology of the oocyte nucleus, e.g. the initiation, completion, and rate of germinal vesicle breakdown (GVBD); the rate and direction of movement of granules in the cytoplasm and nucleus; the cytokinesis of oocyte and first polar body and the movement of and/or duration of the extrusion of the first polar body. Other parameters include the duration of cytokinesis of the mature secondary oocyte and the second polar body.

Examples of cell parameters in a stem cell or population of stem cells that may be assessed by time-lapse imaging include, without limitation, the duration of cytokinesis events, time between cytokinesis events, size and shape of the stem cells prior to and during cytokinesis events, number of daughter cells produced by a cytokinesis event, spatial orientation of the cleavage furrow, the rate and/or number of asymmetric divisions observed (i.e. where one daughter cell maintains a stem cell while the other differentiates), the rate and/or number of symmetric divisions observed (i.e. where both daughter cells either remain as stem cells or both differentiate), and the time interval between the resolution of a cytokinesis event and when a stem cell begins to differentiate.

Parameters can be measured manually, or they may be measured automatically, e.g. by image analysis software. When image analysis software is employed, image analysis algorithms may be used that employ a probabilistic model estimation technique based on sequential Monte Carlo method, e.g. generating distributions of hypothesized embryo/pluripotent cell models, simulating images based on a simple optical model, and comparing these simulations to the observed image data. When such probabilistic model estimations are employed, cells may be modeled as any appropriate shape, e.g. as collections of ellipses in 2D space, collections of ellipsoids in 3D space, and the like. To deal with occlusions and depth ambiguities, the method can enforce geometrical constraints that correspond to expected physical behavior. To improve robustness, images can be captured at one or more focal planes.

C. Gene Expression Analysis

In some embodiments, fragmentation dynamics of embryos are measured in combination with gene expression. Determining the expression of one or more genes, i.e. obtaining an expression profile or expression evaluation, may be made by measuring nucleic acid transcripts, e.g. mRNAs, of the one or more genes of interest, e.g. a nucleic acid expression profile; or by measuring levels of one or more different proteins/polypeptides that are expression products of one or more genes of interest, e.g. a proteomic expression profile. In other words, the terms “expression profile” and “expression evaluation” are used broadly to include a gene expression profile at the RNA level or protein level.

In some embodiments, expression of genes may be evaluated by obtaining a nucleic acid expression profile, where the amount or level of one or more nucleic acids in the sample is determined, e.g., the nucleic acid transcript of the one or more genes of interest. In these embodiments, the sample that is assayed to generate the expression profile is a nucleic acid sample. The nucleic acid sample includes a plurality or population of distinct nucleic acids that includes the expression information of the genes of interest of the embryo or cell being assessed. The nucleic acid may include RNA or DNA nucleic acids, e.g., mRNA, rRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample may be prepared from a single cell, e.g. a pluripotent cell of a culture of pluripotent cells of interest, or a single cell (blastomere) from an embryo of interest; or from several cells, e.g. a fraction of a cultures of pluripotent cells, or 2, 3, or 4, or more blastomeres of an embryo of interest, using standard protocols.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array-based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the level of one or more nucleic acids in a sample may be employed, including those based on amplification protocols, e.g., Polymerase Chain Reaction (PCR)-based assays, including quantitative PCR, reverse-transcription PCR (RT-PCR), real-time PCR, and the like.

In some embodiments, expression of genes may be evaluated by obtaining a proteomic expression profile, where the amount or level of one or more proteins/polypeptides in the sample is determined, e.g., the protein/polypeptide encoded by the gene of interest. In these embodiments, the sample that is assayed to generate the expression profile employed in the methods is a protein sample. Where the expression profile is proteomic expression profile, i.e. a profile of one or more protein levels in a sample, any convenient protocol for evaluating protein levels may be employed wherein the level of one or more proteins in the assayed sample is determined.

While a variety of different manners of assaying for protein levels are known in the art, one representative and convenient type of protocol for assaying protein levels is ELISA. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Such conditions include diluting the sample with diluents such as BSA or bovine gamma globulin (BGG) in phosphate buffered saline (PBS)/Tween or PBS/Triton-X 100, which also tend to assist in the reduction of nonspecific background, and allowing the sample to incubate for about 2-4 hours at temperatures on the order of about 25°-27° C. (although other temperatures may be used). Following incubation, the antisera-contacted surface is washed so as to remove non-immunocomplexed material. An exemplary washing procedure includes washing with a solution such as PBS/Tween, PBS/Triton-X 100, or borate buffer. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. For example, a urease or peroxidase-conjugated anti-human IgG may be employed, for a period of time and under conditions which favor the development of immunocomplex formation (e.g., incubation for 2 hours at room temperature in a PBS-containing solution such as PBS/Tween). After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H2O2, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatograpic column or filter with a wash solution or solvent.

Alternatively, non-ELISA based-methods for measuring the levels of one or more proteins in a sample may be employed. Representative examples include but are not limited to mass spectrometry, proteomic arrays, xMAP™ microsphere technology, flow cytometry, western blotting, and immunohistochemistry.

The resultant data provides information regarding expression for each of the genes that have been probed, wherein the expression information is in terms of whether or not the gene is expressed and, typically, at what level, and wherein the expression data may be both qualitative and quantitative.

In generating the expression profile, in some embodiments a sample is assayed to generate an expression profile that includes expression data for at least one gene/protein, sometimes a plurality of genes/proteins, where by plurality is meant at least two different genes/proteins, and often at least about 3, typically at least about 10 and more usually at least about 15 different genes/proteins or more, such as 50 or more, or 100 or more, etc.

In the broadest sense, the expression evaluation may be qualitative or quantitative. As such, where detection is qualitative, the methods provide a reading or evaluation, e.g., assessment, of whether or not the target analyte, e.g., nucleic acid or expression product, is present in the sample being assayed. In yet other embodiments, the methods provide a quantitative detection of whether the target analyte is present in the sample being assayed, i.e., an evaluation or assessment of the actual amount or relative abundance of the target analyte, e.g., nucleic acid or protein in the sample being assayed. In such embodiments, the quantitative detection may be absolute or, if the method is a method of detecting two or more different analytes, e.g., target nucleic acids or protein, in a sample, relative. As such, the term “quantifying” when used in the context of quantifying a target analyte, e.g., nucleic acid(s) or protein(s), in a sample can refer to absolute or to relative quantification. Absolute quantification may be accomplished by inclusion of known concentration(s) of one or more control analytes and referencing, i.e. normalizing, the detected level of the target analyte with the known control analytes (e.g., through generation of a standard curve). Alternatively, relative quantification can be accomplished by comparison of detected levels or amounts between two or more different target analytes to provide a relative quantification of each of the two or more different analytes, e.g., relative to each other.

Examples of genes whose expression levels are predictive of zygote developmental potential include Cofillin (NM_{—}005507), DIAPH1 (NM_{—}001079812, NM_{—}005219), ECT2 (NM_{—}018098), MYLC2/MYL5 (NM_{—}002477), DGCR8 (NM_{—}022720), Dicer/DICER1 (NM_{—}030621, NM_{—}177438), TARBP2 (NM_{—}004178, NM_{—}134323, NM_{—}134324), CPEB1 (NM_{—}001079533, NM_{—}001079534, NM_{—}001079535, NM_{—}030594), Symplekin/SYMPK (NM_{—}004819), YBX2 (NM_{—}015982), ZAR1 (NM_{—}175619), CTNNB1 (NM_{—}001098209, NM_{—}001098210, NM_{—}001098210, NM_{—}001904), DNMT3B (NM_{—}006892, NM_{—}175848, NM_{—}175849, NM_{—}175850), TERT (NM_{—}198253, NM_{—}198255), YY1 (NM_{—}003403), IFGR2/IFNGR2 (NM_{—}005534), BTF3 (NM_{—}001037637, NM_{—}001207), and NELF (NM_{—}001130969, NM_{—}001130970, NM_{—}001130971, NM_{—}015537). In arriving at a gene expression level measurement, the expression level is often evaluated and then normalized to a standard control, e.g. the expression level in the sample of a gene that is known to be constant through development, e.g. GAPDH or RPLPO, or of a gene whose expression at that timepoint is known.

Gene expression levels may be determined from a single cell, e.g. a blastomere from an embryo of interest, or an isolated oocyte, or an isolated cell from a culture of stem cells, etc., or they may be determined from an embryo, e.g. 2, 3, or 4, or more blastomeres of an embryo of interest, up to and including the whole embryo of interest, or multiple cells from a culture of stem cells, up to and including the whole culture of stem cells, etc.

In other aspects, the present invention comprises a protocol for performing concurrent genotyping and gene expression analysis on a single cell. For embryos, this can be used to improve pre-implantation genetic diagnosis (PGD), a procedure where a single cell is removed from an embryo and its DNA is tested for karyotypic defects or the presence of specific disease genes. Our method allows for concurrent genetic and gene expression analysis. The method involves the following steps: (1) collecting a single cell into a small volume of medium or buffer, (2) performing one-step reverse transcription and polymerase chain reaction (PCR) amplification using a mixture of genotyping and gene expression analysis primers, (3) collecting an aliquot of the amplified cDNA after fewer than 18 cycles of PCR to preserve linearity of the amplification, (4) using the cDNA aliquot to perform gene expression analysis with standard techniques such as quantitative real-time PCR, (5) using the remaining sample to perform a second round of PCR to further amplify the genetic information for genotyping purposes, and (6) genotyping using standard techniques such as gel electrophoresis.

D. Methods of Detecting Aneuploidy

In some embodiments, the embryos are further assessed for their chromosomal content. In some embodiments, the embryos are assessed by monitoring cellular fragmentation dynamics and optionally by measuring one or more other cellular parameters to determine if an embryo is aneuploid. In addition, further evidence of aneuploidy, arising from chromosome sequestering within fragments, can be obtained by immunostaining embryos, e.g., with the centromeric marker, centromere protein-A (CENP-A), the kinetochore and mitotic marker, Aurora B kinase (AURKB), or the nuclear envelope marker, LAMIN-B1, as described herein (see Example 1).

In certain embodiments of the invention, the embryos are found to be aneuploid due to mitotic errors and in other embodiments, the embryos are found to be aneuploid due to meiotic errors. Accordingly, provided herein is a method for ranking embryos from normal to the most severe types of aneuploidy. More specifically, methods are provided for ranking embryos as containing a normal chromosome content, as aneuploid due to mitotic errors, or aneuploid due to meiotic errors based on monitoring fragmentation dynamics (i.e., timing of appearance of embryonic micronuclei or cellular fragmentation) and optionally other cellular parameter measurements.

In some embodiments methods are provided for determining the potential of an embryo to reach blastocyst followed by determining the presence or absence of embryonic micronuclei or fragmentation and/or the level of fragmentation in embryos which have been determined to have blastocyst potential. The presence of embryonic micronuclei or fragmentation, particularly with a high level of fragmentation, is indicative of an aneuploid embryo, and the absence of embryonic micronuclei or fragmentation is indicative of an embryo with a normal chromosome count. A low level of fragmentation is indicative of a lower risk of aneuploidy than embryos with a high level of fragmentation and a higher risk of aneuploidy than embryos with no embryonic micronuclei or fragmentation at all. That is, the lower the level of fragmentation, the less likely the embryo will be aneuploid and the higher the level of fragmentation, the more likely the embryo will be aneuploid. In one embodiment, high fragmentation is characterized by more than about 15% fragmentation by volume of cytoplasm. In still another embodiment, high fragmentation is characterized by more than about 20% fragmentation by volume of cytoplasm. In still another embodiment, high fragmentation is characterized by more than about 25% fragmentation by volume of cytoplasm. In still another embodiment, high fragmentation is characterized by more than about 30% fragmentation by volume of cytoplasm. In another embodiment, low fragmentation is characterized by less than about 30% fragmentation by volume of cytoplasm. In another embodiment, low fragmentation is characterized by less than about 25% fragmentation by volume of cytoplasm. In yet another embodiment, low fragmentation is characterized by less than about 20% fragmentation by volume of cytoplasm. In yet another embodiment, low fragmentation is characterized by less than about 15% fragmentation by volume of cytoplasm.

The method further comprises selecting an embryo with a normal chromosome count by first determining the potential of an embryo to reach the blastocyst stage and then measuring for the presence or absence of embryonic micronuclei or fragmentation and/or the level of fragmentation in an embryo with the potential to reach blastocyst and selecting embryos displaying an absence of embryonic micronuclei or fragmentation or a low level of fragmentation. In addition to the level of fragmentation, fragmentation dynamics of the embryos can also be assessed to determine the likelihood of selecting an embryo with a normal chromosome count. For example, additional fragmentation criteria such as the degree and developmental timing of cellular fragmentation, as measured by time lapse microscopy, or the inclusion of blastomere asymmetry aids in embryo assessment. The potential to reach blastocyst can be measured by determining one or more cellular parameters or by any other method known in the art to be predictive of blastocyst formation. In certain aspects, cellular fragmentation dynamics are measured in combination with one or more other cellular parameters including, the duration of the first cytokinesis, the time interval between cytokinesis 1 and cytokinesis 2, the time interval between cytokinesis 2 and cytokinesis 3, the time until the first cell division, embryo morphology, gene expression patterns, or any other method known in the art to determine the potential of an embryo to reach blastocyst and combinations thereof.

In some embodiments, the embryos determined to be aneuploid are trisomy embryos. A non-limiting list of exemplary trisomies detectable by the methods of the current invention include trisomy 21 (Down syndrome), trisomy 18 (Edwards syndrome), trisomy 13 (Patau syndrome); trisomy 8 (Warkany syndrome 2), trisomy 9, trisomy 16, and trisomy 22 (cat eye syndrome).

In some embodiments, the embryos determined to be aneuploid are monosomy embryos. A non-limiting list of exemplary monosomies detectable by the methods of the current invention include monosomy 22, monosomy 4, monosomy 5, monosomy 7, monosomy 11, monosomy 17 or monosomy X (Turner syndrome).

E. Determining Developmental Potential of an Embryo

Fragmentation dynamics may be used alone or in combination with other methods of embryo assessment, as described herein, to determine the developmental potential of embryos. As discussed above, the terms “developmental potential” and “developmental competence” refer to the ability or capacity of a pluripotent cell or tissue to grow or develop. For example, in the case of an oocyte or embryo, the developmental potential may be the ability or capacity of that oocyte or embryo to grow or develop into a healthy blastocyst. As another example, in the case of a stem cell, the developmental potential is the ability or capacity to grow or develop into one or more cells of interest, e.g. a neuron, a muscle, a B- or T-cell, and the like. In some embodiments, the developmental potential of an oocyte or embryo is the ability or capacity of that ooctye or embryo to develop into a healthy blastocyst; to successfully implant into a uterus; to go through gestation; and/or to be born live. In some embodiments, the developmental potential of a pluripotent cell is the ability or capacity of that pluripotent cell to develop into one or more cells of interest, e.g. a neuron, a muscle, a B- or T-cell, and the like; and/or to contribute to a tissue of interest in vivo.

By “good developmental potential” or “favorable developmental potential”, it is meant that the embryo/pluripotent cell is statistically likely to develop as desired, i.e., it has a 55%, 60%, 70%, 80%, 90%, 95% or more chance, e.g., a 100% chance, of developing as desired. In other words, 55 out of 100, 60 out of 100, 70 out of 100, 80 out of 100, 90 out of 100, 95 out of 100, or 100 out of 100 embryos or pluripotent cells demonstrating the cell parameter measurements used to arrive at the determination of favorable developmental potential do, in fact, go on to develop as desired. Conversely, by “poor developmental potential’ it is meant that the embryo/pluripotent cell is not statistically likely to develop as desired, i.e. it has a 50%, 40%, 30%, 20%, 10%, 5% or less chance, e.g. 0% chance, of developing as desired. In other words, only 50 out of 100, 40 out of 100, 30 out of 100, 20 out of 100, 10 out of 100, or 5 out of 100 or less of the embryos or pluripotent cells demonstrating the cell parameter measurements used to arrive at the determination of poor developmental potential do, in fact, go on to develop as desired. As used herein, “normal’ or “healthy’ embryos and pluripotent cells demonstrate favorable developmental potential, whereas “abnormal’ embryos and pluripotent cells display poor developmental potential.

In some embodiments, a cell parameter measurement is used directly to determine the developmental potential of the embryo. In other words, the absolute value of the measurement itself is sufficient to determine the developmental potential. Examples of this in embodiments using time-lapse imaging to measure cell parameters include, without limitation, the following, any of which alone or in combination are indicative of favorable developmental potential in a human embryo: (a) a cytokinesis 1 that lasts about 0-30 minutes, for example, about 6-20 minutes, on average about 14.3±6.0 minutes; (b) a cell cycle 1 that lasts about 20-27 hours, e.g. about 25-27 hours; (c) a time interval between the resolution of cytokinesis 1 and the onset of cytokinesis 2 that is about 8-15 hours, e.g. about 9-13 hours, with an average value of about 11.1+/−2.1 hours; (d) a time interval, i.e. synchronicity, between the initiation of cytokinesis 2 and the initiation of cytokinesis 3 that is about 0-5 hours, e.g. about 0-3 hours, with an average time of about 1.0+/−1.6 hours. Examples of direct measurements, any of which alone or in combination are indicative of poor developmental potential in a human embryo, include without limitation: (a) a cytokinesis 1 that lasts longer than about 30 minutes, for example, about 32, 35, 40, 45, 50, 55, or 60 minutes or more; (b) a cell cycle 1 that lasts longer than about 27 hours, e.g. 28, 29, or 30 or more hours; (c) a time interval between the resolution of cytokinesis 1 and the onset of cytokinesis 2 that last more than 15 hour, e.g. about 16, 17, 18, 19, or 20 or more hours, or less than 8 hours, e.g. about 7, 5, 4, or 3 or fewer hours; (d) a time interval between the initiation of cytokinesis 2 and the initiation of cytokinesis 3 that is 6, 7, 8, 9, or 10 or more hours.

In some embodiments, the cell parameter measurement is employed by comparing it to a cell parameter measurement from a reference, or control, embryo/pluripotent cell, and using the result of this comparison to provide a determination of the developmental potential of the embryo/pluripotent cell. The terms “reference” and “control” as used herein mean a standardized embryo or cell to be used to interpret the cell parameter measurements of a given embryo and assign a determination of developmental potential thereto. The reference or control may be an embryo/pluripotent cell that is known to have a desired phenotype, e.g., favorable developmental potential, and therefore may be a positive reference or control embryo. Alternatively, the reference/control embryo/pluripotent cell may be an embryo known to not have the desired phenotype, and therefore be a negative reference/control embryo.

In certain embodiments, the obtained cell parameter measurement(s) is compared to a comparable cell parameter measurement(s) from a single reference/control embryo to obtain information regarding the phenotype of the embryo/cell being assayed. In yet other embodiments, the obtained cell parameter measurement(s) is compared to the comparable cell parameter measurement(s) from two or more different reference/control embryos or pluripotent cells to obtain more in depth information regarding the phenotype of the assayed embryo/cell. For example, the obtained cell parameter measurements from the embryo(s) or pluripotent cell(s) being assessed may be compared to both a positive and negative embryo or pluripotent cell to obtain confirmed information regarding whether the embryo/cell has the phenotype of interest.

As an example, cytokinesis 1 in a normal human embryo, i.e. with favorable developmental potential, is about 0-30 minutes, more usually about 6-20 minutes, on average about 14.3±6.0 minutes, i.e. about 1, 2, 3, 4, or 5 minutes, more usually about 6, 7, 8, 9, 10, 11, 12, 13, 14, 15, 16, 17, 18, 19, or 20 minutes, in some cases 21, 22, 23, 24, 25, 26, 27, 28, 29, or up to about 30 minutes. A longer period of time to complete cytokinesis 1 in the embryo being assessed as compared to that observed for a normal reference embryo is indicative of poor developmental potential. As a second example, cell cycle 1 in a normal embryo, i.e. from the time of fertilization to the completion of cytokinesis 1, is typically completed in about 20-27 hours, more usually in about 25-27 hours, i.e. about 15, 16, 17, 18, or 19 hours, more usually about 20, 21, 22, 23, or 24 hours, and more usually about 25, 26 or 27 hours. A cell cycle 1 that is longer in the embryo being assessed as compared to that observed for a normal reference embryo is indicative of poor developmental potential. As a third example, the resolution of cytokinesis 1 and the onset of cytokinesis 2 in normal human embryos is about 8-15 hours, more often about 9-13 hours, with an average value of about 11.1+/−2.1 hours; i.e. 6, 7, or 8 hours, more usually about 9, 10, 11, 12, 13, 14 or up to about 15 hours. A longer or shorter cell cycle 2 in the embryo being assessed as compared to that observed for a normal reference embryo is indicative of poor developmental potential. As a fourth example, the time interval between the initiation of cytokinesis 2 and the initiation of cytokinesis 3, i.e. the synchronicity of the second and third mitosis, in normal human embryos is usually about 0-5 hours, more usually about 0, 1, 2 or 3 hours, with an average time of about 1.0+/−1.6 hours; a longer interval between the completion of cytokinesis 2 and cytokinesis 3 in the embryo being assessed as compared to that observed in a normal reference embryo is indicative of poor developmental potential. Finally, as an example of how this embodiment may be applied when using gene expression levels as parameters for assessing developmental potential, lower expression levels of Cofillin, DIAPH1, ECT2, MYLC2, DGCR8, Dicer, TARBP2, CPEB1, Symplekin, YBX2, ZAR1, CTNNB1, DNMT3B, TERT, YY1, IFGR2, BTF3 and/or NELF, i.e. 1.5-fold, 2-fold, 3-fold, 4-fold, 5-fold, 10-fold, 20-fold, 50-fold, or 100-fold lower expression, in 2-cell embryos being assessed as compared to that observed for a normal reference 2-cell embryo is indicative of poor developmental potential, whereas expression that is equal to or greater than that observed for a normal reference 2-cell embryo is indicative of favorable developmental potential. Other examples may be derived from empirical data, e.g. by observing one or more reference embryos alongside the embryo to be assessed. Any reference embryo may be employed, e.g. a normal reference sample with favorable developmental potential, or an abnormal reference sample with poor developmental potential. In some cases, more than one reference sample may be employed, e.g. both a normal reference sample and an abnormal reference sample may be used.

In some embodiments, it may be desirable to use cell parameter measurements that are arrived at by time-lapse microscopy or by expression profiling, but not by both time-lapse microscopy and expression profiling. In other embodiments, it may be desirable to use cell parameter measurements that are arrived at by time-lapse microscopy as well as cell parameter measurements that are arrived at by expression profiling.

As discussed above, fragmentation dynamics alone or in combination with one or more other parameters may be measured and employed to determine the developmental potential of an embryo. In some embodiments, a measurement of fragmentation dynamics alone may be sufficient to arrive at a determination of developmental potential. In some embodiments, it may be desirable to employ measurements of fragmentation dynamics in combination with one or more other cell parameters, for example, 1 cell parameter, 2 cell parameters, 3 cell parameters, or 4 or more cell parameters.

In certain embodiments, assaying for multiple parameters may be desirable as assaying for multiple parameters may provide for greater sensitivity and specificity. By sensitivity it is meant the proportion of actual positives which are correctly identified as being such. This may be depicted mathematically as:

$$Sensitivity = \frac{( Number of true positives )}{( Number of true positives + Number of false negatives )}$$

Thus, in a method in which “positives” are the embryos that have favorable developmental potential, i.e. that will develop into blastocysts, and “negatives” are the embryos that have poor developmental potential, i.e. that will not develop into blastocysts, a sensitivity of 100% means that the test recognizes all embryos that will develop into blastocysts as such. In some embodiments, the sensitivity of the assay may be about 70%, 80%, 90%, 95%, 98% or more, e.g. 100%. By specificity it is meant the proportion of negatives which are correctly identified as such. This may be depicted mathematically as

$$Specificity = \frac{( Number of true positives )}{( Number of true negatives + Number of false positives )}$$

Thus, in a method in which positives are the embryos that have favorable developmental potential, i.e. that will develop into blastocysts, and negatives are the embryos that have poor developmental potential, i.e. that will not develop into blastocysts, a specificity of 100% means that the test recognizes all embryos that will not develop into blastocysts, i.e. will arrest prior to the blastocyst stage, as such. In some embodiments, the specificity of the assay may be about 70%, 80%, 90%, 95%, 98% or more, e.g. 100%.

F. Determining Aneuploidy or Chromosomal Content from Image Analysis

Fragmentation dynamics may also be used alone or in combination with other methods of embryo assessment, as described herein, to determine the aneuploidy status and/or chromosomal content of the embryo. As stated above “aneuploidy” refers to embryos with an abnormal chromosome content, including but not limited to those caused by mitotic errors or meiotic errors, including for example, trisomies, monosomies and mosaicism.

By “normal” or “normal chromosome count” is meant that the embryo contains the proper number of pair chromosomes for the species. For example, a “normal” human embryo will contain a pair of 23 chromosomes for a total of 46 chromosomes.

In some embodiments, a cell parameter measurement is used in combination with fragmentation dynamics to determine the aneuploidy status and/or chromosomal count of an embryo. Examples of cell parameters that may be used to aid in the determination of aneuploidy status include, the following: (a) a cytokinesis 1 that is outside the normal range of about 0 to about 30 minutes; (b) the time interval between cytokinesis 1 and cytokinesis 2 that is outside the normal range of about 8 to about 15 hours; (c) the time interval between cytokinesis 2 and cytokinesis 3 that is outside the normal range of about 0 to about 5 hours. Specifically, any one of the following alone or in combination are indicative of aneuploidy: (a) a cytokinesis 1 that is greater than about 30 minutes; (b) a time interval between cytokinesis 1 and cytokinesis 2 that is less than about 8 hours; and/or (c) a time interval between cytokinesis 2 and cytokinesis 3 that is greater than about 90 minutes.

In some embodiments the cell parameter measurement can be used in addition to monitoring fragmentation dynamics to determine if the detected aneuploidy is a result of a mitotic or a meiotic error. For example, one or more of the following cellular measurements indicate that the detected aneuploidy arises due to mitotic errors: (a) a duration of cytokinesis that is longer than about 35 minutes, for example about 35.5, 36, 38, 40, 42, 44, 46, 48, 50, 55, 60, 65, 70, 75, 80, 85, 90, 95 or 100 minutes or more; (b) a time interval between cytokinesis 1 and cytokinesis 2 that is shorter than about 7 hours, for example about 6.5, 6, 5, 4, 3, 2, or 1 hour or less; and/or a (c) a time interval between cytokinesis 2 and cytokinesis 3 that is longer than about 2 hours, for example about 2.5, 3, 4, 5, 6, 7, 8, or 9 hours or more. Specifically, the following measurements either alone or in combination are particularly indicative of an aneuploidy due to mitotic error: (a) a duration of the first cytokinesis that is about 36.0±66.9 minutes; (b) a time interval between cytokinesis 1 and cytokinesis 2 that is about 6.4±6.6 hours; and/or a (c) a time interval between cytokinesis 2 and cytokinesis 3 that is about 2.0±3.9 hours. In another example, one or more of the following cellular measurements alone or in combination indicate that the detected aneuploidy arises due to meiotic errors: (a) a duration of cytokinesis that is longer than about 100 minutes, for example about 105, 125, 150, 175, 200, 225, 250, 275, 300, 325, 350, 375, or 400 minutes or more; (b) a time interval between cytokinesis 1 and cytokinesis 2 that is shorter than about 4 hours, for example about 3.5, 3, 2, or 1 hour or less; and/or a (c) a time interval between cytokinesis 2 and cytokinesis 3 that is longer than about 2 hours, for example about 2.5, 3, 4, 5, 6, 7, 8, or 9 hours or more. Specifically, the following measurements either alone or in combination are particularly indicative of an aneuploidy due to mitotic error: (a) a duration of the first cytokinesis that is about 117.2±166.5 minutes; (b) a time interval between cytokinesis 1 and cytokinesis 2 that is about 4.0±5.2 hours; and/or a (c) a time interval between cytokinesis 2 and cytokinesis 3 that is about 2.0±4.3 hours. In another example, the cellular parameter measurements are used to rank the embryos in increasing levels of severity from normal embryos, to aneuploid embryos where the aneuploidy is due to one or more mitotic errors, to aneuploid embryos where the aneuploidy is due to one or more meiotic errors.

In some embodiments, fragmentation dynamics are used in combination with one or more cell parameter measurements to select one or more embryos with a normal chromosome count. For example, time lapse imaging may be used to monitor fragmentation dynamics (i.e., to determine timing of the appearance of any embryonic micronuclei or fragmentation) in combination with measuring cell parameters. The absence of any embryonic micronuclei or fragmentation in combination with measurements indicating that cell parameters fall in a normal range can be used to select embryos having a normal chromosome count. Cell parameters indicating that an embryo has a normal chromosome count include the following: (a) a cytokinesis 1 that lasts about 0-30 minutes, for example, about 6-20 minutes, on average about 14.4±4.2 minutes; (b) a cell cycle 1 that lasts about 20-27 hours, e.g. about 25-27 hours; (c) a time interval between the resolution of cytokinesis 1 and the onset of cytokinesis 2 that is about 8-15 hours, e.g. about 9-13 hours, with an average value of about 11.8+/−0.71 hours; (d) a time interval, i.e. synchronicity, between the initiation of cytokinesis 2 and the initiation of cytokinesis 3 that is about 0-5 hours, e.g. about 0-3 hours, with an average time of about 0.96+/−0.84 hours. In another embodiment, the one or more embryos selected by the cell parameter measurement because of a normal chromosome count, is provided to a female in need thereof.

G. Reporting Developmental Potential and/or Chromosomal Abnormalities

In some embodiments, the assessment of an embryo includes generating a written report that includes the artisan's assessment of the subject embryo, e.g. a “developmental potential assessment,” an “assessment of chromosomal abnormalities,” etc. Thus, a subject method may further include a step of generating or outputting a report providing the results of such an assessment, which report can be provided in the form of an electronic medium (e.g., an electronic display on a computer monitor), or in the form of a tangible medium (e.g., a report printed on paper or other tangible medium).

A “report,” as described herein, is an electronic or tangible document which includes report elements that provide information of interest relating to an assessment arrived at by methods of the invention. A subject report can be completely or partially electronically generated. A subject report includes at least an assessment of the developmental potential of the subject embryo, an assessment of the probability of the existence of chromosomal abnormalities, etc. A subject report can further include one or more of: 1) information regarding the testing facility; 2) service provider information; 3) subject data; 4) sample data; 5) a detailed assessment report section, providing information relating to how the assessment was arrived at, e.g. a) analysis of fragmentation dynamics and optionally other cell parameter measurements taken, b) reference values employed, if any; and 6) other features.

The report may include information about the testing facility, which information is relevant to the hospital, clinic, or laboratory in which sample gathering and/or data generation was conducted. Sample gathering can include how the sample was generated, e.g. how it was harvested from a subject, and/or how it was cultured etc. Data generation can include how images were acquired or gene expression profiles were analyzed. This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the user.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample preparation and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include a subject data section, including medical history of subjects from which oocytes or pluripotent cells were harvested, patient age, in vitro fertilization or intracytoplasmic sperm injection cycle characteristics (e.g. fertilization rate, day 3 follicle stimulating hormone (FSH) level), and, when oocytes are harvested, zygote/embryo cohort parameters (e.g. total number of embryos). This subject data may be integrated to improve embryo assessment and/or help determine the optimal number of embryos to transfer. The report may also include administrative subject data (that is, data that are not essential to the assessment of developmental potential) such as information to identify the subject (e.g., name, subject date of birth (DOB), gender, mailing and/or residence address, medical record number (MRN), room and/or bed number in a healthcare facility), insurance information, and the like), the name of the subject's physician or other health professional who ordered the assessment of developmental potential and, if different from the ordering physician, the name of a staff physician who is responsible for the subject's care (e.g., primary care physician).

The report may include a sample data section, which may provide information about the biological sample analyzed in the assessment, such as the type of sample (embryo or pluripotent cell, and type of pluripotent cell), how the sample was handled (e.g. storage temperature, preparatory protocols) and the date and time collected. Report fields with this information can generally be populated using data entered by the user, some of which may be provided as pre-scripted selections (e.g., using a drop-down menu).

The report may include an assessment report section, which may include information relating to how the assessments/determinations were arrived at as described herein. The interpretive report can include, for example, time-lapse images of the embryo being assessed, and/or gene expression results. The assessment portion of the report can optionally also include a recommendation(s) section. For example, where the results indicate favorable developmental potential of an embryo, the recommendation can include a recommendation that a limited number of embryos be transplanted into the uterus during fertility treatment as recommended in the art.

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide the analysis requested by the user (e.g., an assessment of developmental potential).

H. Applications

As discussed above, methods of the invention may be used to assess embryos, to determine their developmental potential, to detect aneuploidy, to select embryos with normal chromosome counts and/or to rank embryos based on the type of aneuploidy. These determinations may be used to guide clinical decisions and/or actions. For example, in order to increase pregnancy rates, clinicians often transfer multiple embryos into patients, potentially resulting in multiple pregnancies that pose health risks to both the mother and fetuses. Using results obtained from the methods of the invention, the developmental potential of embryos being transferred to develop into fetuses is determined prior to transplantation, allowing the practitioner to decide how many embryos to transfer so as to maximize the chance of success of a full term pregnancy while minimizing risk. Additionally, the methods of the invention can be used to select embryos for implantation that have a normal chromosome count, that are not aneuploid, so as to not only increase pregnancy rates, but also to decrease miscarriage rates and decrease rates of non-lethal aneuploidy births, for example, by being able to select against embryos which may have lethal chromosomal abnormalities, such as, for example trisomy 16 or non-lethal abnormalities such as trisomy 21.

Assessments made by following methods of the invention may also find use in ranking embryos in a group of embryos for their developmental potential. For example, in some instances, multiple embryos may be capable of developing into blastocysts, i.e. will have favorable developmental potential. However, some embryos will be more likely to achieve the blastocysts stage or a higher-quality blastocyst than others, i.e. they will have better developmental potential than other embryos. In such cases, methods of the invention may be used to rank the embryos in the group. In such methods, cellular fragmentation dynamics and optionally one or more other cell parameters for each embryo are measured for each embryo, and used to determine the developmental potential of the embryos relative to one another. In some embodiments, the measurements for each of the embryos are compared directly to one another to determine the developmental potential of the embryos. In some embodiments, the measurements for each of the embryos are compared to a reference embryo to determine the developmental potentials for each embryo, and then developmental potentials for each embryo are compared to determine the developmental potential of the embryos relative to one another. In this way, a practitioner assessing, for example, multiple zygotes/embryos, can choose only the best quality embryos, i.e. those with the best developmental potential, to transfer so as to maximize the chance of success of a full term pregnancy while minimizing risk.

Similarly, the methods of the invention may also find use in ranking embryos based on their chromosomal content. For example, in some instances multiple embryos will be found to be aneuploid. However, some of the aneuploidies will be less severe than others. For example, aneuploidy caused by errors in mitotic cell division is generally less severe than an aneuploidy caused by errors in meiotic cell division. In such cases, methods of the invention may be used to rank embryos in the group. In such methods, cellular fragmentation dynamics and optionally one or more other cellular parameters for each embryo are measured for each embryo. The measurements from each of the embryos are used to determine whether or not the embryo is aneuploid and if it is aneuploid whether that aneuploidy is the less severe aneuploidy resulting from one or more mitotic errors or the more severe aneuploidy resulting from one or more meiotic errors. In some embodiments, the measurements for each of the embryos are compared directly to one another to determine the type/severity of the aneuploidy of the embryos. In some embodiments, the measurements for each of the embryos are compared to a reference embryo to determine the aneuploidy type/severity for each embryo, and then the determined aneuploidy for each embryo is compared to determine the aneuploidy type/severity of the embryos relative to one another. In this way, a practitioner assessing, for example, multiple zygotes/embryos, can choose only the best quality embryos, i.e. those that are normal or with less severe types of aneuploidy, to transfer so as to maximize the chance of success of a full term pregnancy while minimizing risk.

Assessments made by following the methods of the invention may also find use in determining the developmental potential of oocytes that are matured in vitro and stem cells that are cultured in vitro. Information on the developmental potential of oocytes obtained by the methods of the invention can guide the practitioner's selection of ooctyes to fertilize, resulting in higher probability of success in deriving blastocysts from these oocytes. Likewise, information on the developmental potential of stem cells can inform the practitioner's selection of stem cells to use in procedures to, e.g. reconstitute or replace a tissue in vivo in a subject in need thereof.

I. Reagents, Devices and Kits

Also provided are reagents, devices and kits thereof for practicing one or more of the above-described methods. The subject reagents, devices and kits thereof may vary greatly. Reagents and devices of interest include those mentioned above with respect to the methods of measuring any of the aforementioned cell parameters, where such reagents may include culture plates, culture media, microscopes, imaging software, imaging analysis software, nucleic acid primers, arrays of nucleic acid probes, antibodies, signal producing system reagents, etc., depending on the particular measuring protocol to be performed. For example, reagents may include PCR primers that are specific for one or more of the genes Cofillin, DIAPH1, ECT2, MYLC2/MYL5, DGCR8, Dicer/DICER1, TARBP2, CPEB1, Symplekin/SYMPK, YBX2, ZAR1, CTNNB1, DNMT3B, TERT, YY1, IFGR2/IFNGR2, BTF3, and NELF, as described above. Other examples of reagents include arrays that comprise probes that are specific for one or more of the genes of interest, or antibodies to the proteins encoded by these genes of interest.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Dynamic Blastomere Behavior Reflects Human Embryo Ploidy by the Four-Cell Stage

Introduction

Efforts to elucidate key pathways of normal human embryo development and the factors that contribute to abnormal development, embryo arrest and aneuploidy have benefited from recent advances in imaging, molecular and genetic analyses of single blastomeres and whole embryos (Wong et al. (2010) Nature Biotechnol. 28:1115-1121; Dobson et al. (2004) Hum. Mol. Genet. 13:1461-1470; Zhang et al. (2009) PLoS One 4: e7844; Galan et al. (2010) PLoS ONE 5:e13615; Vanneste et al. (2009) Nature Med. 15:577-583). Previously, we and our collaborators used non-invasive time-lapse imaging to identify three dynamic cell cycle parameters that predict success or failure of human embryos to develop to blastocyst stage, by the four cell stage (Wong et al., supra). The three predictive parameters are the duration of the first cytokinesis, the time from the two- to three-cell stage and the synchronicity in the appearance of the third and fourth embryonic blastomeres. Analysis of gene expression profiles indicated that embryos that are predicted to develop to the blastocyst stage differ significantly in gene expression from those that arrest prior to blastocyst formation, suggesting that non-invasive imaging is an indicator of molecular health in embryos (Wong et al., supra).

Considering that the incidence of human embryonic aneuploidy is high (approximately 50-80%) and in light of the dynamic nature of the cell cycle parameters that predict successful development to the blastocyst stage, we contemplated whether non-invasive time-lapse imaging of dynamic human embryonic blastomere behavior might provide a means to distinguish euploid from aneuploid embryos. To assess the feasibility of non-invasive imaging for the detection of human embryonic aneuploidy, we focused on the analysis of aneuploidy generated prior to embryonic genome activation, in the first four cells of the human embryo. We analyzed a unique set of embryos that were cryopreserved at the 1-cell stage prior to assessment of quality and thus, likely to be representative of embryos typically obtained in the IVF clinic. Such embryos have also been shown to have similar potential for successful development, implantation, pregnancy and delivery (Wong et al., supra; Veeck et al. (1993) Feral. Steril. 59:1202-1207; Miller & Goldberg (1995) Obst. Gynecol. 85:999-1002; El-Toukhy et al. (2003) Hum. Reprod. 18:1313-1318). We focused on human embryo imaging given that mouse embryos do not commonly exhibit widespread aneuploidy during early development (Lightfoot et al. (2006) Dev. Biol. 289:384-394). By correlating noninvasive time-lapse imaging with the chromosomal composition of all blastomeres in each human embryo, we demonstrate that cell cycle parameters in conjunction with dynamic fragmentation analysis largely reflect underlying ploidy to the 4-cell stage and that the high frequency of human embryonic aneuploidy (Vanneste et al., supra; Johnson et al. (2010) Hum. Reprod. 25:1066-1075) may have contributions from chromosome-containing fragments/micronuclei that arise during the cleavage stage of human development.

Methods

Sample Source and Procurement

Approximately 85 2 pronuclear (2PN) and 25 cleavage-stage supernumerary human embryos from successful IVF cycles, subsequently donated for non-stem research, were obtained with written informed consent from the Stanford University RENEW Biobank. De-identification and molecular analysis was performed according to the Stanford University Institutional Review Board-approved protocol #10466 entitled “The RENEW Biobank”. No protected health information was associated with individual embryos; approximately 25% of couples used donor gametes. The average maternal age was 33.5 years old, and the most common cause of infertility was unexplained for 35.4% of couples.

Human Embryo Thawing and Culture

Human embryos frozen at the zygotic or 2PN stage were thawed by a 2-step rapid thawing protocol using Quinn's Advantage Thaw Kit (Cooper Surgical, Trumbull, Conn.) as previously described (Wong et al., supra). In brief, either cryostraws or vials were removed from the liquid nitrogen and exposed to air before incubating in a 37° C. water bath. Once thawed, embryos were transferred to a 0.5 mol/L sucrose solution for 10 minutes followed by a 0.2 mol/L sucrose solution for an additional 10 minutes. The embryos were then washed in Quinn's Advantage Medium with Hepes (Cooper Surgical) plus 5% Serum Protein Substitute (SPS; CooperSurgical) and each transferred to a 60 μl microdrop of Quinn's Advantage Cleavage Medium (CooperSurgical) supplemented with 10% SPS under mineral oil (Sigma, St. Louis, Mo.) and cultured at 37° C. with 6% CO₂, 5% O₂ and 89% N₂, standard human embryo culture conditions in accordance with current clinical IVF practice. Embryos were cultured in custom polystyrene petri dishes with 25 individual microwells located in the center to help track embryo identity during imaging and subsequent handling. Each microwell is 250 microns wide and 100 microns deep and accommodates a single developing embryo. To maintain group culture, all of the micro-wells share a common media drop, which is stabilized by an extruded ring. Small fiducial markers (letters and numbers) are located near the micro-wells for embryo identification.

Time-Lapse Imaging and Parameter Analysis

Embryo development was monitored using a custom-built miniature microscope system that can be placed on the shelf of a conventional incubator. The system consists of 2 inverted digital microscopes, each with LED illumination, 10× Olympus objective, manual focus knob and 5 megapixel CMOS camera. The microscopes were modified for darkfield illumination by placing a darkfield aperture between the collimated white LED and the condenser lens as previously described (Wong et al, supra). Images were taken at a 0.6 second exposure time every 5 minutes for up to 2 days (approximately 30 hours) until the majority of embryos reached the 4-cell stage. The microscopes were connected to an external PC via USB cables that were passed through the rear access port of the incubator. A custom software program (written in C++) was used to control the microscopes, provide a user interface and save the images to file. After each experiment, images were compiled into a time-lapse movie with well identification labels and timestamps that allowed manual measurement of the imaging parameters. The time interval for each cell cycle parameter was measured by three independent evaluators prior to the completion of the ACGH and micronuclei analysis to ensure blinded parameter measurements.

Array-Comparative Genomic Hybridization (A-CGH)

Following imaging, the embryos were transferred to Acidified Tyrode's Solution (Millipore) to remove the zona pellucida (ZP) and ZP-free embryos were disaggregated in Quinn's Advantage Calcium and Magnesium free Medium with Hepes plus 10% Human Albumin (Cooper Surgical). Once disaggregated, each embryonic blastomere was washed three times in 10 μl drops of nonstick wash buffer and transferred to a sterile PCR tube. DNA extraction and pre-amplification was accomplished using the SurePlex Kit according to the manufacturer's instructions (BlueGnome). In brief, the DNA was extracted from each sample as well as the reference sample with Cell Extraction Enzyme at 75° C. for 10 minutes and the DNA was denatured and pre-amplified with PicoPlex Pre-Amp Enzyme by a 95° C. hotstart for 2 minutes and 12 cycles of gradient PCR and then with PicoPlex Amplification Enzyme at 95° C. for 2 minutes and 14 cycles of 95° C. for 15 seconds, 65° C. for 1 minute and 75° C. for 1 minute. Following whole genome amplification, each sample was fluorescently labeled with either Cy3 or Cy5 and hybridized to the BlueGnome CytoChip, which is a BAC array with greater than 5000 replicated clones designed to detect submicroscopic copy number variations, and covers approximately 30% of the human genome (cytochip.com). We chose A-CGH instead of alternatives for several reasons: First, ACGH with the BlueGnome platform has now been validated in a large number of studies in reproductive clinics and genetic testing centers, including the detection of admixtures of chromosomally-normal and aneuploidy cell lines, single cell analysis of embryos that were ultimately transferred and comparisons with other standard STR methods (Mamas et al. (2012) Fertil. Steril. 97:943-947; Fiorentino et al. (2011) Hum. Reprod. 26:1925-1935; Fishel et al. (2011) J. Fertil. In Vitro 1). Second, although the use of SNP arrays would also be very applicable, there is less information regarding the reliability of single cell analysis and in many cases, inclusion of parental DNA is used to interpret many of the SNPs and eliminate potential complications such as allele drop out. Third, the genome coverage provided by A-CGH appeared sufficient to detect aneuploidies of every chromosome reliably. Scanned images were analyzed and chromosomal copy number ratios quantified and reported using the CytoChip algorithm and BlueFuse software (BlueGnome). Threshold levels and whole chromosomal losses or gains were determined by 3 times the standard deviation, greater than ±0.3 log₂ ratio call, or both as previously described (Gutierrez-Mateo et al. (2011) Fertil. Steril. 95:953-958).

Confocal Imaging Analysis and 3-Dimensional Modeling

The ZP was removed by treatment with Acidified Tyrode's Solution and ZP-free embryos were washed three times in Phosphate Buffered Solution (PBS; Invitrogen, Carlsbad) with 0.1% Bovine Serum Albumin (BSA; Sigma-Aldrich) and 0.1% Tween-20 (PBS-T; Sigma-Adrich;) before fixation in 4% paraformaldehyde in PBS (USB Corp., Cleveland, Ohio) for 20 minutes at Room Temperature (RT). Once fixed, the embryos were washed three times in PBS-T to remove any residual fixative and stained with 1 μg/ml DAPI and 0.5 μg/ml MitoTracker Red CMXRos (Invitrogen) for 15 minutes at RT. For the visualization of CENP-A and LAMIN-B1, zona-free embryos were fixed in 100% cold methanol at −20° C. for 15 minutes to preserve each epitope, washed and then permeabilized in 1% Triton X-100 (Sigma-Aldrich) for 1 hour at RT. Following permeabilization, the embryos were washed three times in PBS-T and then blocked in 4% normal donkey serum (Jackson ImmunoReasearch Laboratories, Inc., West Grove, Pa.) in PBS-T overnight at 4° C. The embryos were incubated w/ primary antibodies in PBS-T with 1% donkey serum sequentially for 1 hour each at RT at the following dilutions: 1 μg/ml CENP-A [a kind gift from Ben Moree in Aaron Straight's laboratory at Stanford University] and 1:1,000 rabbit LAMIN-B1 (Abcam; catalog #ab16048). Primary signals were detected using the appropriate 488, 568 or 647-conjugated donkey Alexa Fluor secondary antibody (Invitrogen) at a 1:250 dilution at RT for 1 hour in the dark. Immunofluorescence was visualized by sequential imaging, whereby the channel track was switched at the same focal plane each frame to avoid cross-contamination between channels set at 1 airy unit, using a Zeiss LSM510 Meta inverted laser scanning confocal microscope described here: nisms.stanford.edu/Equipment/LSM510Meta01v01.html. Confocal sections were captured at 1 mm intervals throughout the whole embryo and processed in ImageJ (NIH) for Z-stack imaging analysis. Three-dimensional reconstructions of embryos were accomplished with IMARIS (Bitplane).

Fluorescent In Situ Hybridization (FISH)

FISH was performed by first incubating in a 1% tri-sodium citrate hypotonic buffer prior to fixation in Carnoy's solution (3:1 ratio of methanol to glacial acetic acid) and then mounting on poly-L-lysine treated slides (Fisher Scientific, Pittsburgh, Pa.) matured overnight at 60° C. Hybridization to chromosome 18 (CEP 18/D18Z1/Spectrum Orange) and the Y-chromosome (CEP Y/DYZ1/Spectrum Aqua) was completed using fluorescently labeled DNA probes (Abbott Molecular, Des Plaines, Ill.) for 2 days in a 37° C. humidified chamber and positive signals were visualized by epifluorescent microscopy and DAPI staining by confocal microscopy. FISH signals for chromosome 16 (CEP 16/D16Z3/Spectrum Green) and chromosome 21 (21q22.13-22.2/LSI 21/Spectrum Orange) were visualized with DAPI staining by confocal microscopy.

Statistical Analysis

The individual parameter data is represented as the average±the standard deviation and analyzed for statistical significance (p<0.05) using both one-way analysis of variance (ANOVA) with the Bonferonni multiple comparisons post-test and the non-parametric test, Kruskal-Wallis, with Dunn's post-test. Fisher's Exact Test was used to calculate the sensitivity and specificity of predicting aneuploidy with the parameters.

Results

Experimental Design for Assessing Human Blastomere Behavior

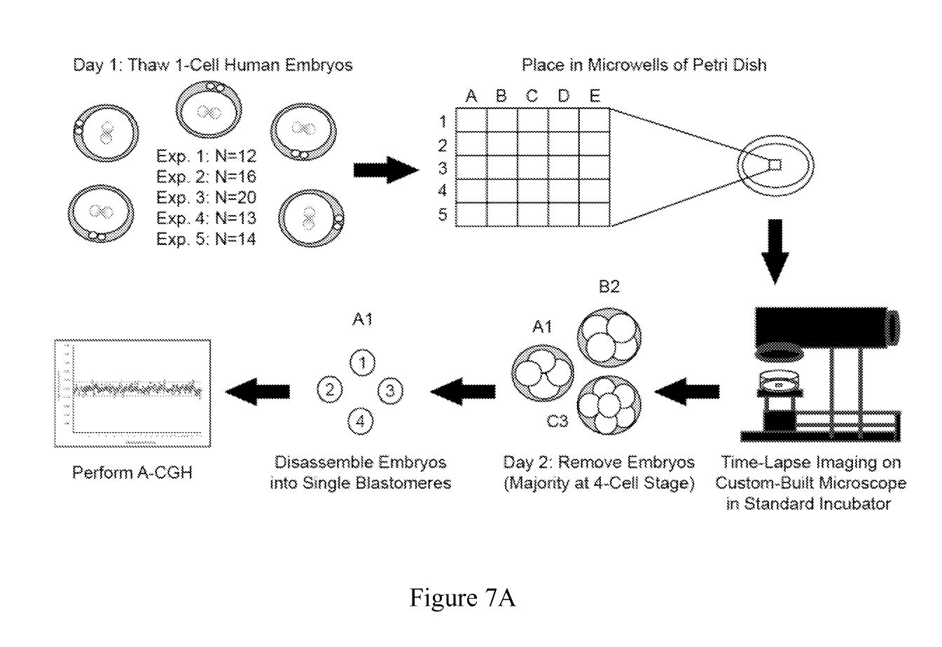

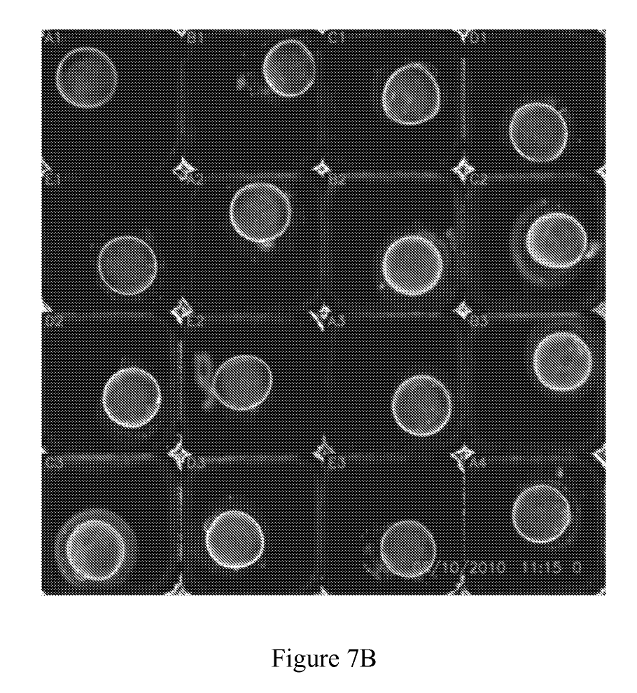

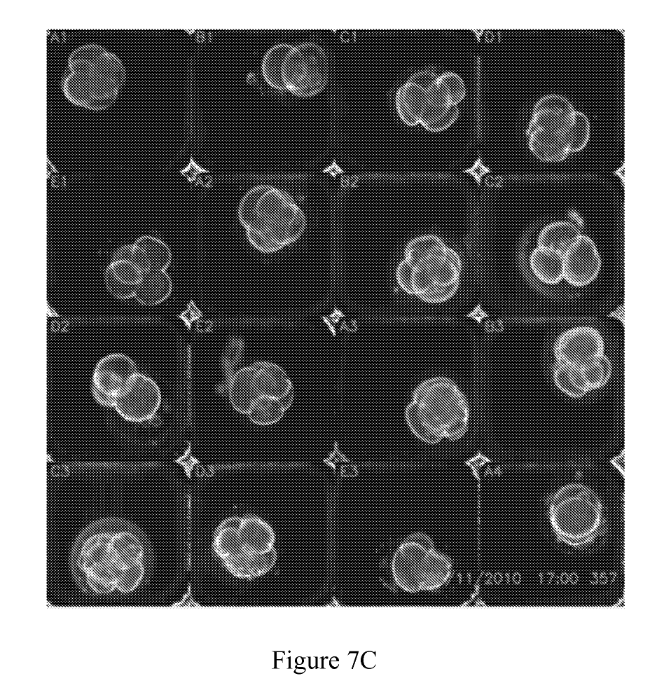

The experimental design used to investigate the relationship between cell cycle parameters and ploidy in human embryos up to the 4-cell stage is as shown (FIG. 7A). Seventy-five human zygotes were cultured for 2 days and embryonic development was recorded to 4-cells by time-lapse imaging with image collection every 5 minutes. This experimental design allowed us to capture key cell cycle parameters and reconstruct embryo karyotypes, a task that is difficult given the complex aneuploidy and high rates of mosaicism in human blastomeres by the 8-cell stage (Vanneste et al., supra; Johnson et al. (2010) Hum. Reprod. 25, 1066-1075; Kuo et al. (1998) J. Assist. Reprod. Genet. 15:276-280; Baart et al. (2006) Hum. Reprod. 21:223-233). Images from each experiment were then compiled into time-lapse movies with microwell labels and timestamps that allowed manual measurement of imaging parameters (FIGS. 7B and 7C). Time intervals for each cell cycle parameter were measured by three independent evaluators.

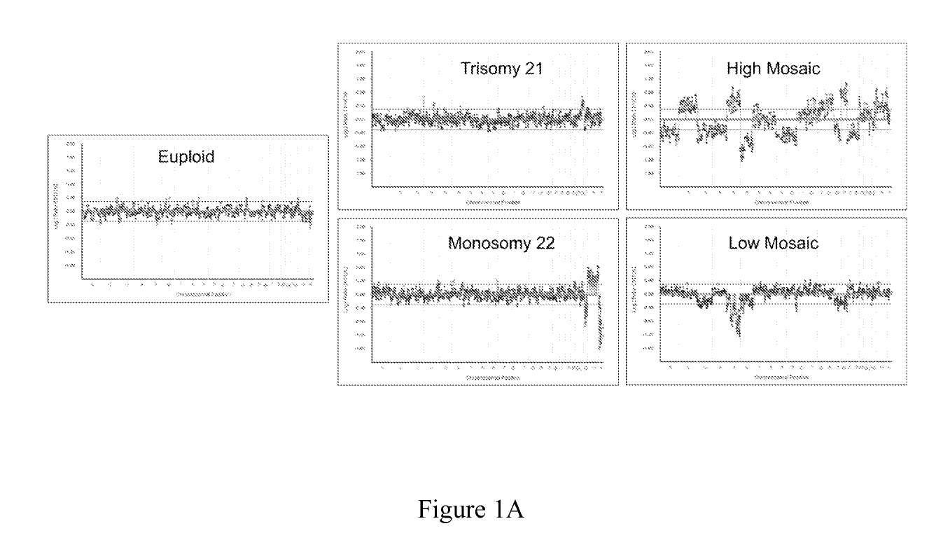

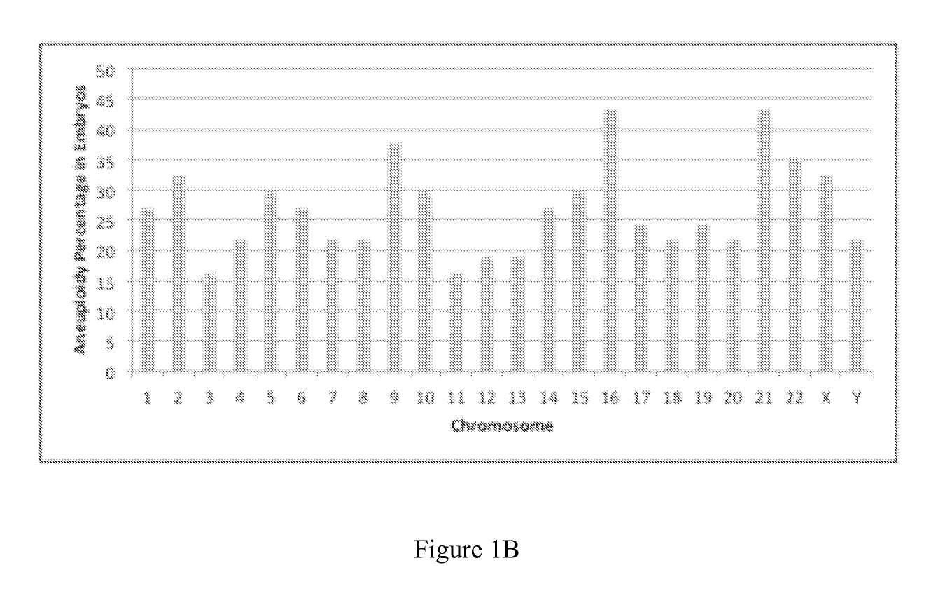

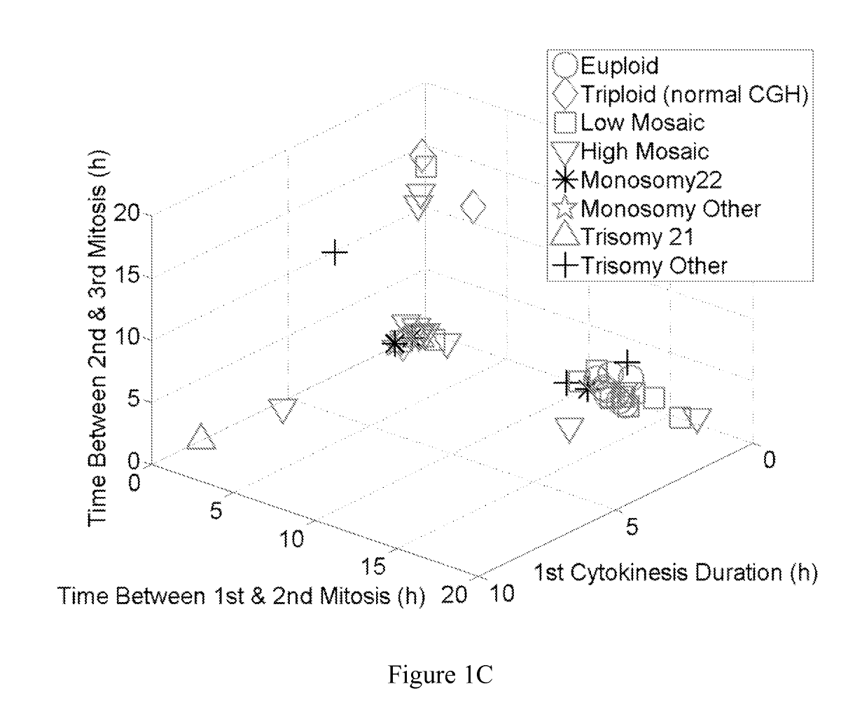

We observed that 53 of the 75 embryos progressed beyond the zygote stage as is typical of human development (Table 1a). These embryos were disassembled for analysis of ploidy in each individual blastomere via 24-chromosome Array-Comparative Genomic Hybridization (A-CGH; FIG. 7A). Eight embryos were excluded from analysis for technical reasons such as cell lysis (Table 1a), resulting in the analysis of 185 blastomeres from 45 embryos: 8 euploid, 34 aneuploid and 3 embryos which appeared to be triploid (Table 1a) based on previous observations of a distinct dynamic morphological signature of dividing from a single cell to three daughter cells in the first cleavage division (Wong et al., supra). The observation that approximately 75% of the human embryos analyzed by A-CGH were aneuploid is consistent with previous studies using whole genome approaches (Vanneste et al., supra; Johnson et al., supra). We also observed that the surprisingly high frequency of aneuploidy was not preferentially associated with a subset of chromosomes—all 22 pairs of autosomes and both sex chromosomes were affected (FIG. 1B). We also observed that in most cases (29 of 34), aneuploid embryos did not contain a single euploid blastomere capable of contributing to the embryo proper later in development (Table 1a).

Detection of Meiotic and Mitotic Errors by the 4-Cell Stage

Based on the results of chromosome analysis, we next sought to differentiate between types of errors in each aneuploid embryo. For this purpose, we inferred that: 1) aneuploid 4-cell embryos characterized by different chromosome compositions between blastomeres had incurred mitotic errors and 2) embryos with identical aneuploidies in all blastomeres to the 4-cell stage had most likely inherited meiotic errors. From these inferences, we concluded that 20% of 4-cell embryos ( 9/45) exhibited meiotic errors and >50% of the embryos ( 25/45) displayed mitotic errors, suggesting that the generation of chromosomal errors during early human embryo development may be most frequently mitotic in origin (Table 1a). Of the nine embryos with meiotic errors, we identified two embryos with trisomy 21, one of the most common types of autosomal trisomy that survives to birth, resulting in Down syndrome, and three embryos with monosomy 22, which is incompatible with live birth (FIG. 1A). In the embryos with mitotic errors (Table 2), we observed simple mitotic mosaicism, in which the same single chromosome was affected in the blastomeres of some embryos, but the majority ( 20/25) exhibited complex mosaicism, or an abnormal number of multiple chromosomes within one or more blastomeres of an embryo (Table 1a)

Cell Cycle Parameter Timing in Euploid and Aneuploid Embryos

Next, we examined the mean values and standard deviations of cell cycle parameters in embryos that were determined to be chromosomally normal and observed complete overlap with previous reports (Wong et al., supra) but with smaller standard deviations: (i) 14.4±4.2 minutes duration of the first cytokinesis from the visualization of the cleavage furrow to the appearance of two distinct blastomeres, (ii) 11.8±0.71 hours from 2-cells to the appearance of a 3^{rd} cell, and (iii) 0.96±0.84 hours time from 3-cells to 4-cells (Table 1b). When we examined aneuploid embryos, we determined that embryos with meiotic errors (N=9) exhibited a much greater standard deviation than euploid embryos in all three parameters (*=p<0.05 by ANOVA; p=0.06 by Kruskal-Wallis), including the duration of the first cytokinesis. In contrast, a higher standard deviation in the time interval from 2- to 3-cells and the synchronicity in the appearance of the 3^{rd} and 4^{th} cells (*=p<0.05 by ANOVA; P=0.08 by Kruskal-Wallis) was observed in embryos with mitotic errors (N=25), especially those with high mitotic mosacism (N=13, **=p<0.01 by ANOVA; *=p<0.05 by Kruskal-Wallis). The calculated sensitivity and specificity of the parameters when used to predict euploidy by Fisher's Exact Test was 100% and 66%, respectively, given that all embryos predicted to be aneuploid by imaging behavior were actually chromosomally abnormal (p<0.001).

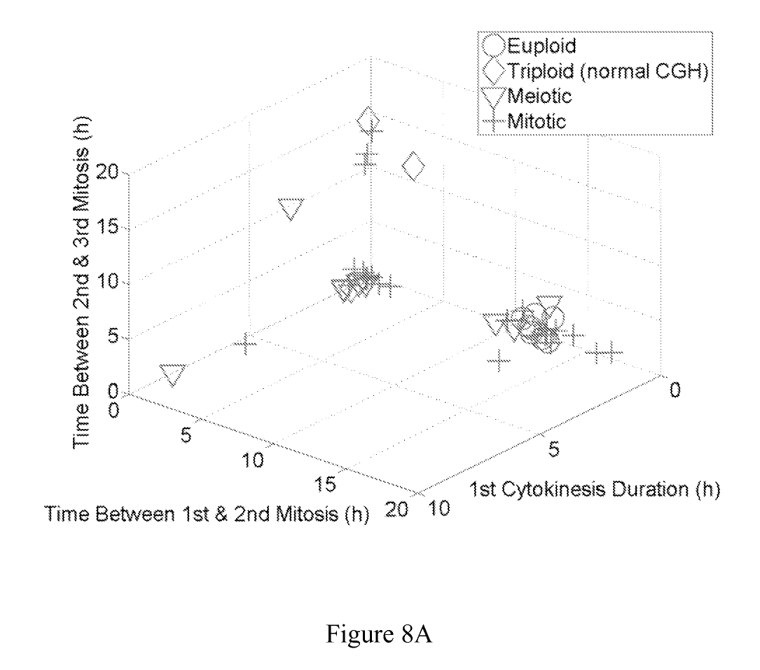

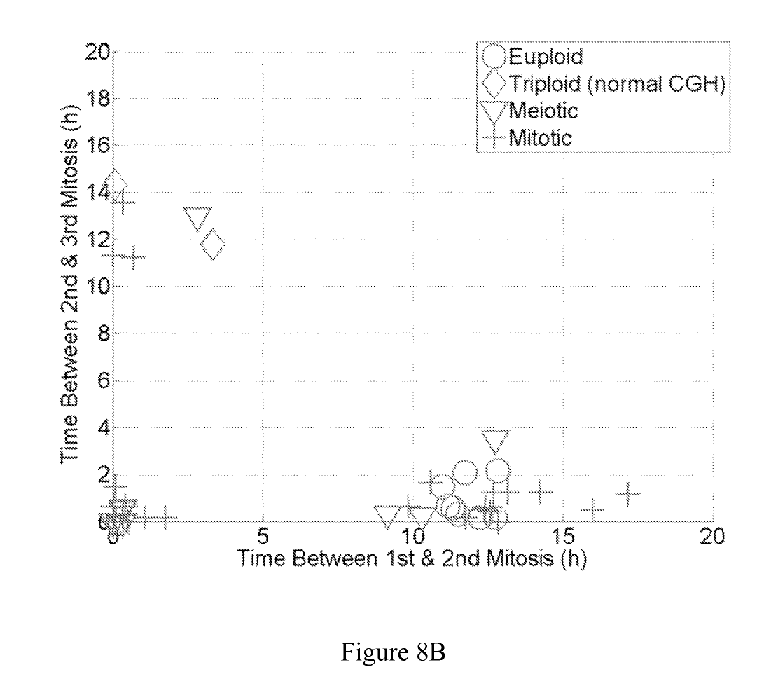

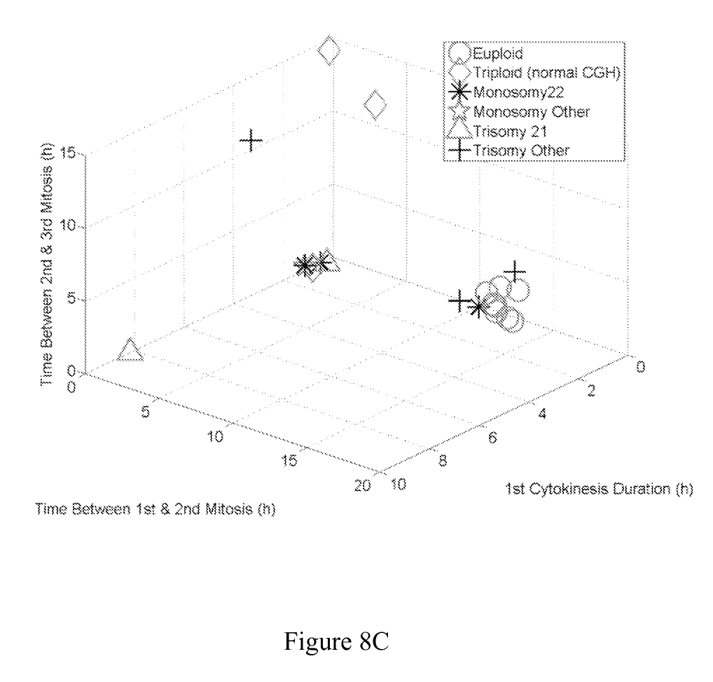

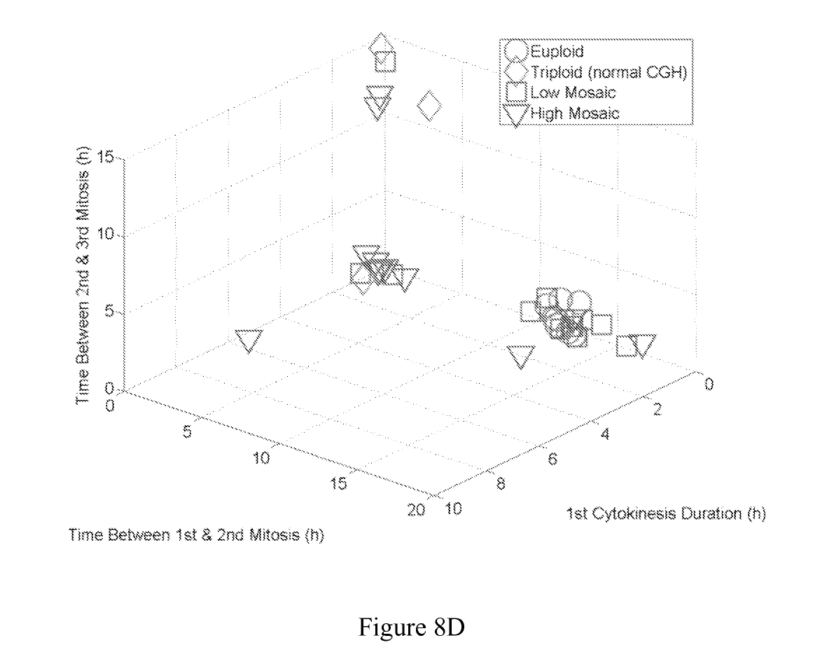

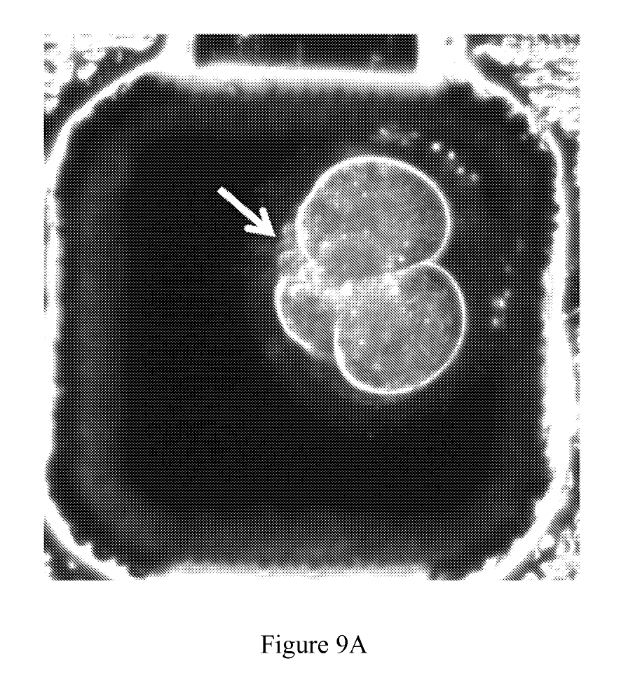

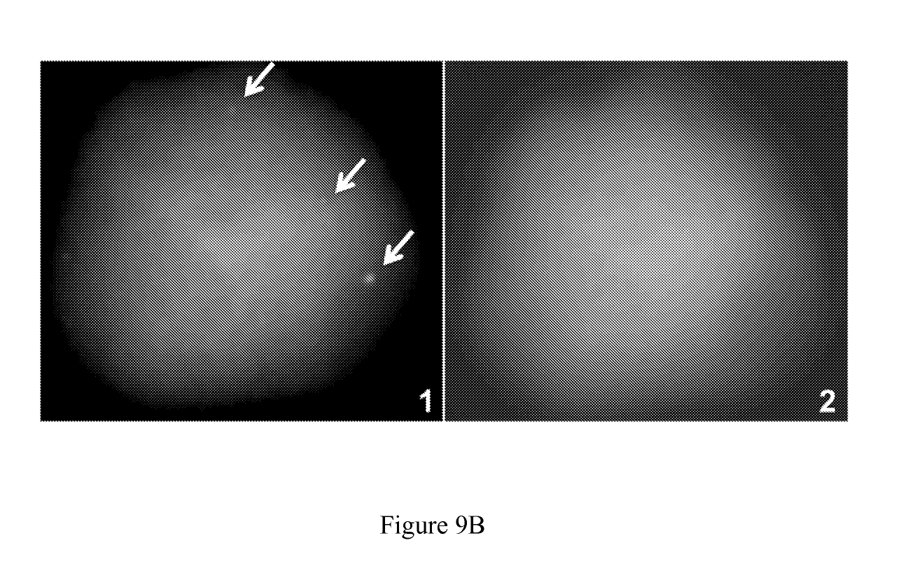

We next graphed each embryo as a point in a 3-dimensional plot, where each dimension represented a cell cycle parameter. As shown (FIG. 1C), the majority of embryos with normal karyotypes clustered together in a region nearly identical to non-arrested or developmentally normal embryos as previously observed (Wong et al., supra). In contrast, approximately 70% of the aneuploid embryos exhibited parameter values outside the timing windows of euploid embryos and, therefore, concentrated in other areas away from the euploid embryo clustering when graphed. While 11 of 25 embryos with mitotic errors concentrated in a similar two- and three-dimensional area as embryos with normal A-CGH profiles, the set of embryos with meiotic errors exhibited more sporadic parameter clustering in plots (FIG. 1C and FIG. 8A-8C). Embryos that appeared to be triploid (Table 1a), on the other hand, accumulated close to zero for the second parameter in 3-D plots (FIG. 1C). We verified triploidy via fluorescent in situ hybridization (FISH) of chromosome 18 in a single blastomere of an embryo that appeared to be triploid during time-lapse imaging analysis (FIGS. 9A and 9B). This observation further indicated that alterations in ploidy are reflected in cell cycle parameters of developing human embryos.

Distinction Between Low/High-Degree Mitotic Mosaicism

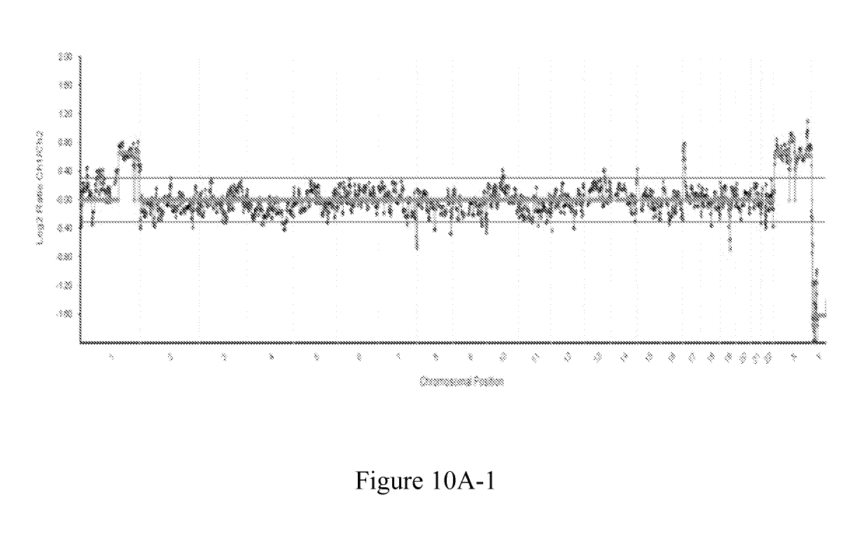

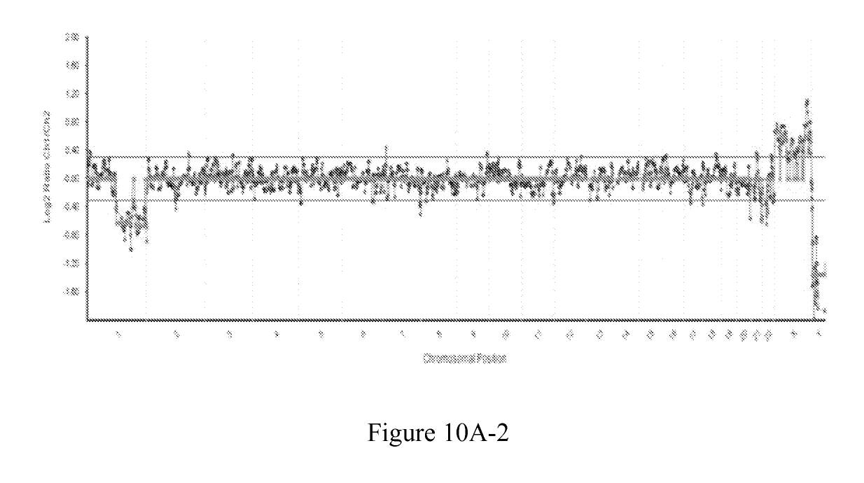

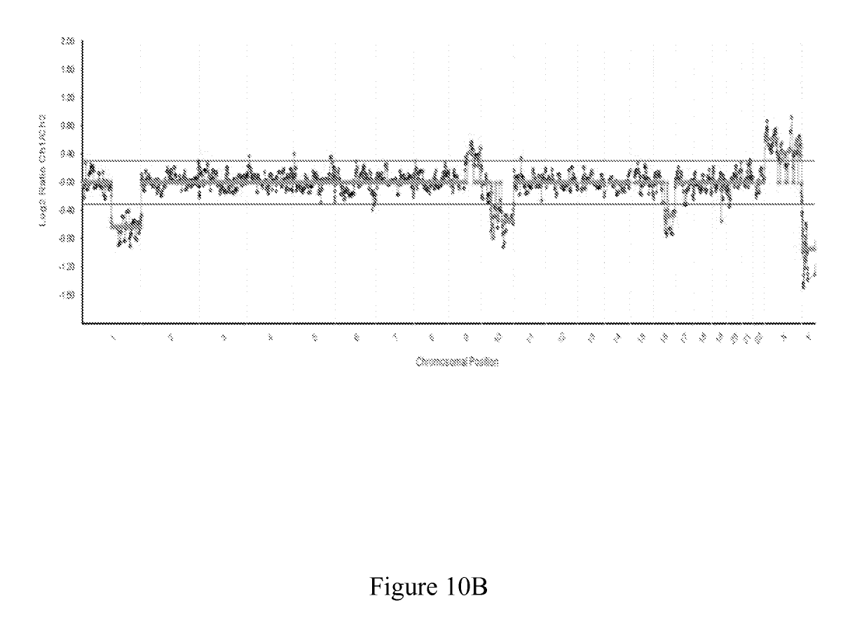

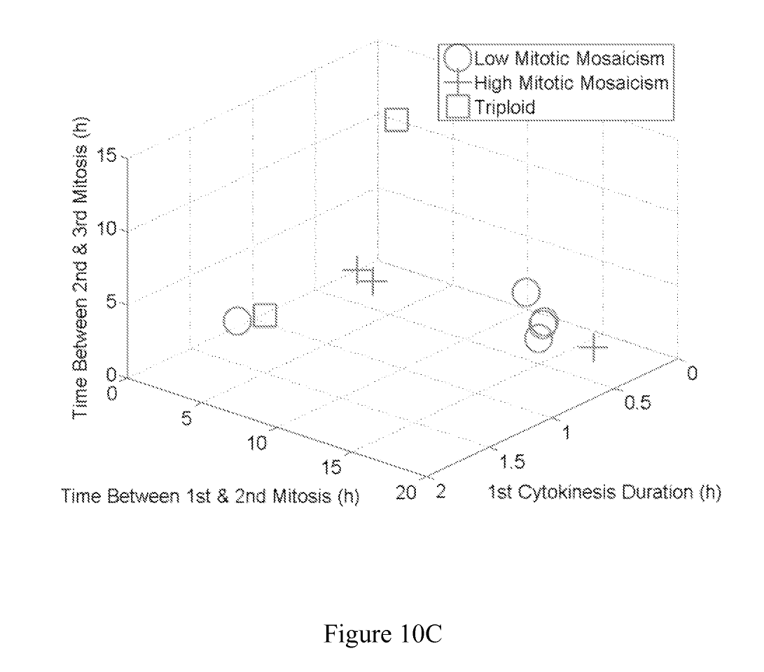

Given the overlap of parameters of a subset of embryos with mitotic errors with those of euploid embryos (FIG. 8B), we further examined the relationship between the chromosomal composition of each blastomere and cell cycle characteristics. Embryos that had blastomeres with a low degree of mitotic mosaicism were associated with tighter clustering of cell cycle parameters near euploid embryos; a shift in parameter timing appeared to occur when four or fewer chromosomes were segregated abnormally (FIG. 1C, FIG. 7D and Table 2). Thus, we classified embryos with defects in four chromosomes or fewer as “low mosaic” and designated embryos with more than four chromosomes affected as “high mosaic” (FIG. 1A). The term “mosaic” is used here to refer to the presence of multiple aneuploidies within a single blastomere rather than mosaicism between blastomeres. Individual parameter analysis revealed that while low mosaic embryos had cell cycles more similar to embryos with normal karyotypes, high mosaic embryos deviated significantly, particularly in the time between the first and second mitosis (Table 1b). Of the embryos with mitotic errors, we also determined that eight, in addition to two triploid embryos, had sub-chromosomal losses and/or gains, most of which were unbalanced (Table 3 and FIG. 10). Thus, our findings to this point indicated differences in cell cycle parameters associated with the generation of different types of chromosomal abnormalities, including partial chromosome losses and/or gains.

Correlation Between Cellular Fragmentation and Aneuploidy

Under current IVF clinical practice, developmental competence of human embryos is assessed most commonly on Day 3 or Day 5 of culture based on relatively simple morphological characteristics that may include blastomere number, blastomere symmetry and the degree of cellular fragmentation (Antczak & Van Blerkom (1999) Hum. Reprod. 14:429-447; Alikani et al. (1999) Fertil. Steril. 71, 836-842; Ebner et al. (2001) Fertil. Steril. 76, 281-285; Racowsky et al. (2011) Fertil. Steril. 95, 1985-1989). Cellular fragmentation, or the generation of what is commonly thought to be cytoplasmic fragments, occurs frequently in human embryos (Antczak & Van Blerkom, supra), and is distinct from DNA fragmentation associated with cell death late in pre-implantation development (Hardy et al. (2001) Proc. Natl. Acad. Sci. 98, 1655-1660; Spanos et al. (2002) Reproduction 124:353-363). There is evidence to suggest that embryo fragmentation occurs in vivo, not just in vitro (Pereda & Croxatto (1978) Biol. Reprod. 18:481-489; Buster et al. (1985) Am. J. Obstet. Gyn. 153:211-217), and that it may be negatively correlated with implantation potential (Alikani et al. (1999) Fertil. Steril. 71:836-842; Pelinck et al. (2010) Fertil. Steril. 94:527-534)

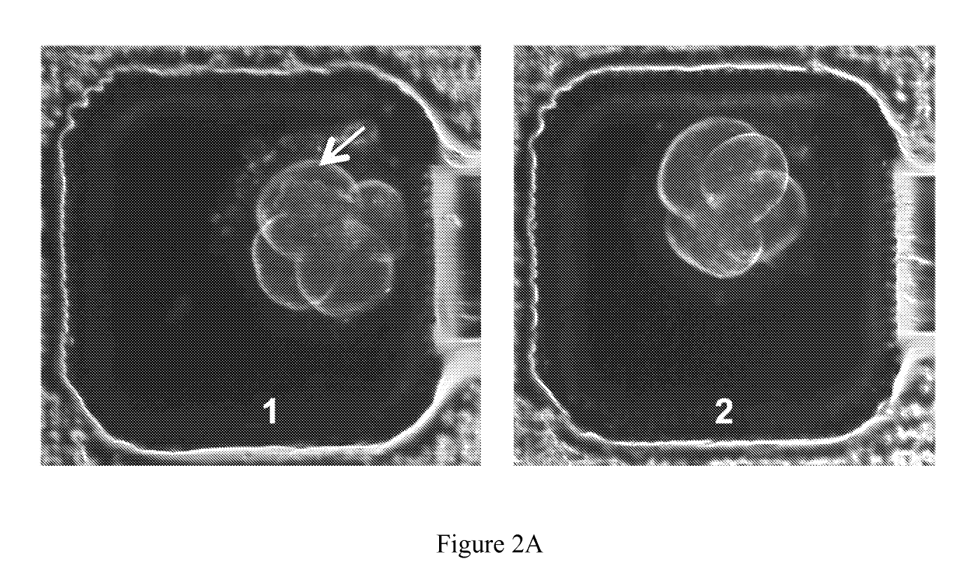

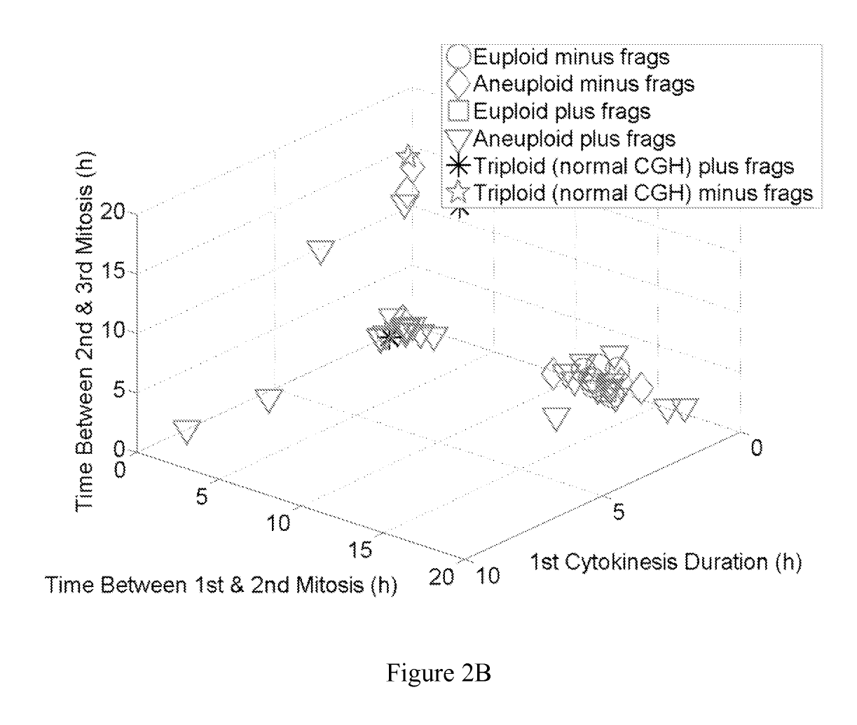

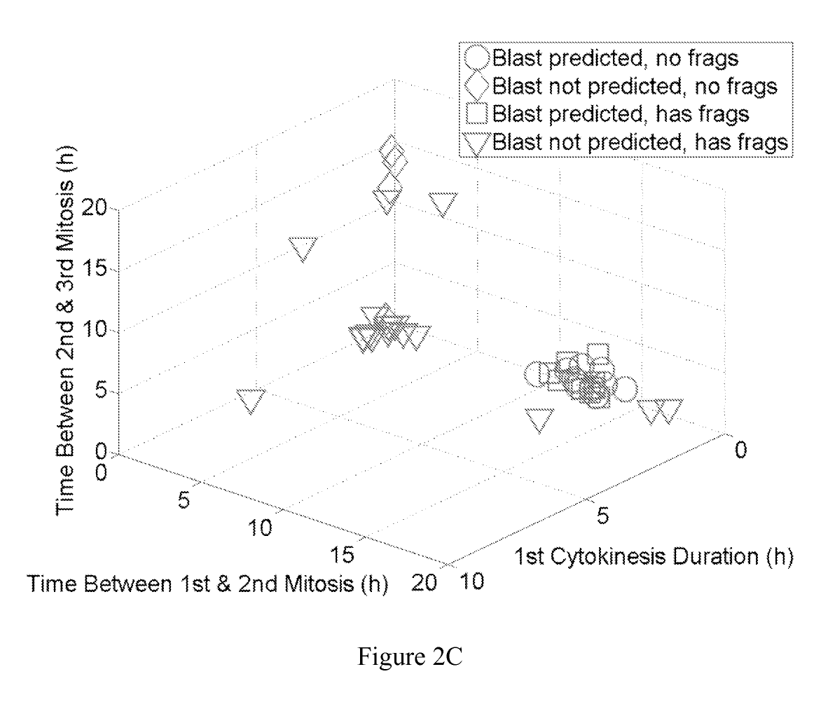

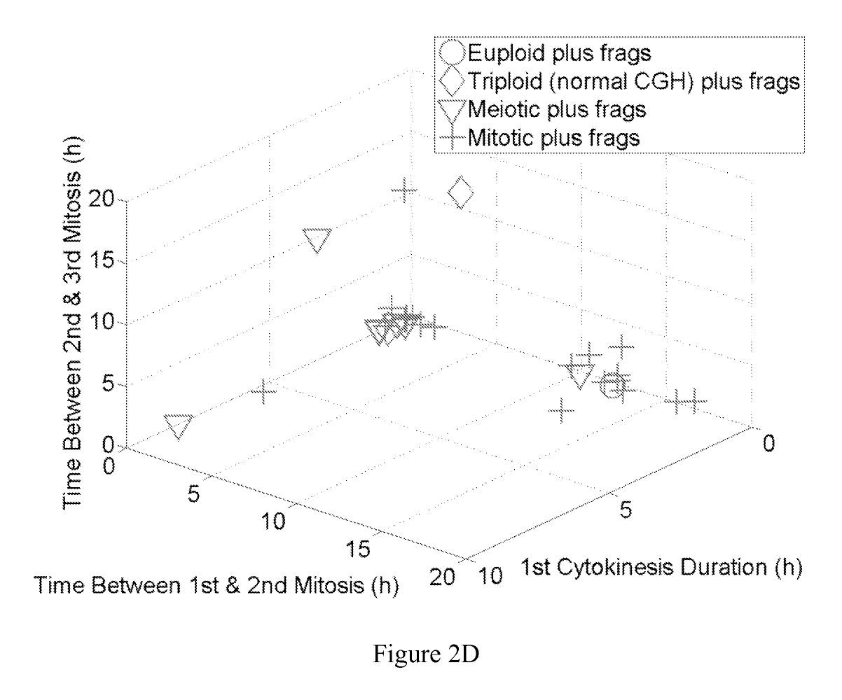

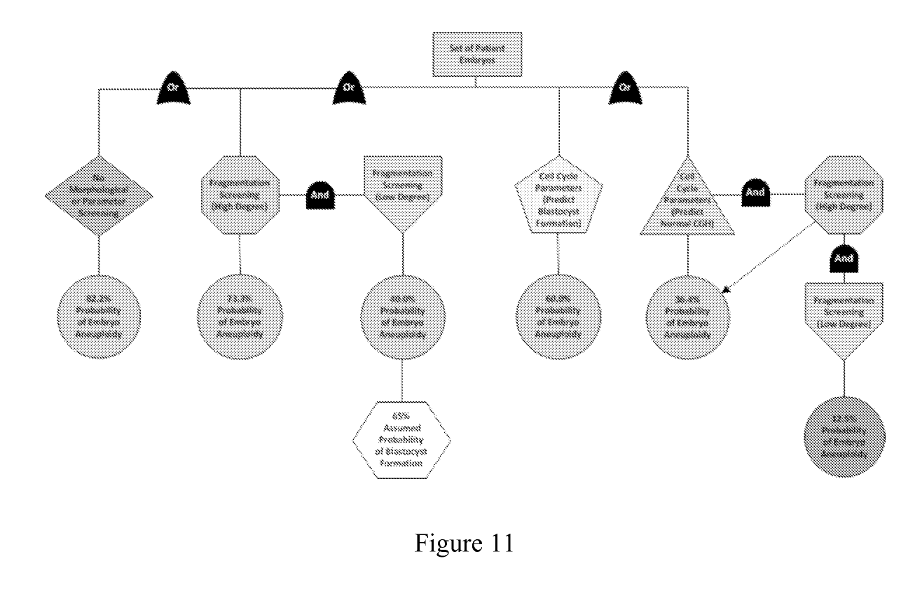

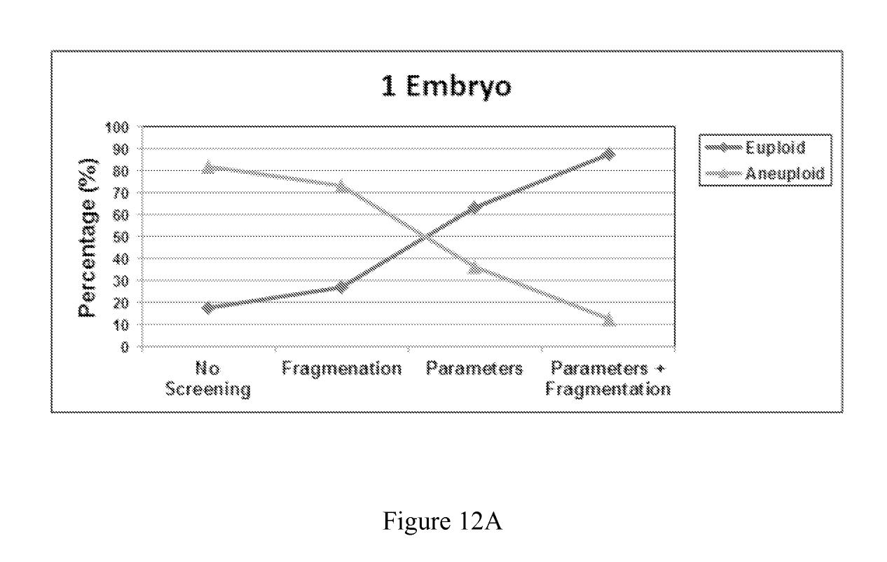

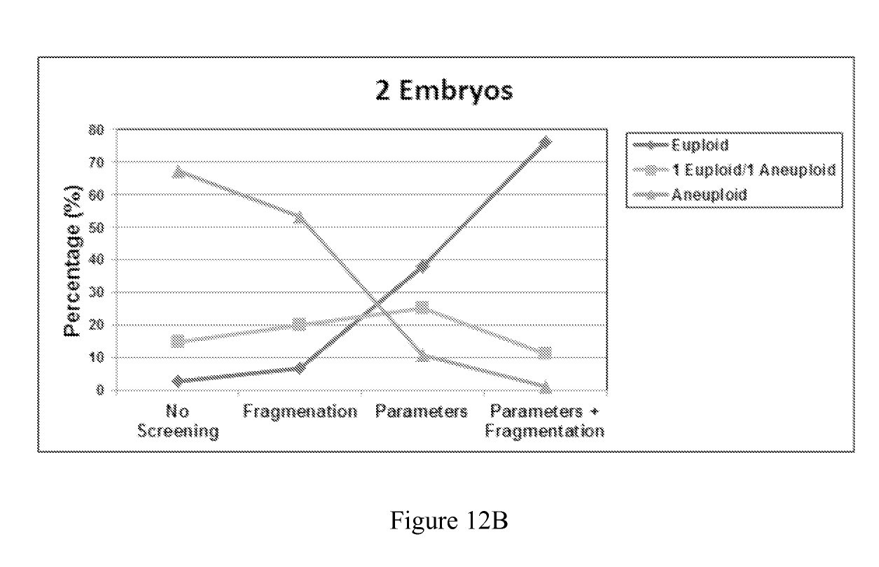

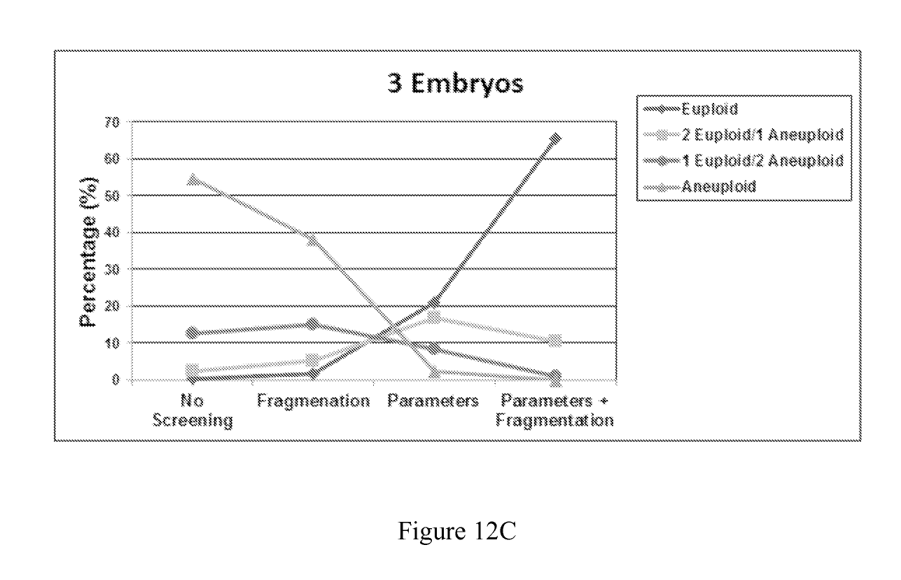

In examining the time-lapse images and dynamic behaviors of human embryos, we observed that a large proportion of aneuploid and triploid, but not euploid embryos, exhibited cellular fragmentation (Table 1c). When we analyzed cell cycle imaging parameters in embryos that did and did not exhibit fragmentation (FIGS. 2A1 and 2A2), we determined that dynamic assessment of fragmentation might assist in distinguishing euploid and aneuploid embryos with parameters that clustered together (FIG. 2B). However, only 65% of the embryos characterized by the presence of fragments would be predicted to form blastocysts (FIG. 2C), a value in accordance with the findings of Wong et al. (Nature Biotechnol. (2010) 28:1115-1121). In addition, fragmentation screening in the absence of other morphological and/or dynamic criteria had minimal effect on the probability of embryonic euploidy in comparison to inclusion of analysis of cell cycle parameters (FIG. 11 and Table 4). This was exemplified by calculating the probability of embryonic euploidy versus aneuploidy using fragmentation and/or parameter screening criteria if 1, 2 or 3 embryos were selected (FIG. 12 and Table 5).

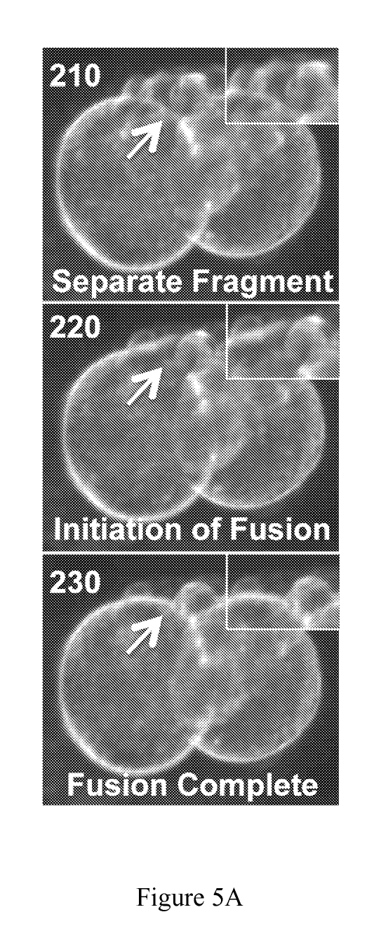

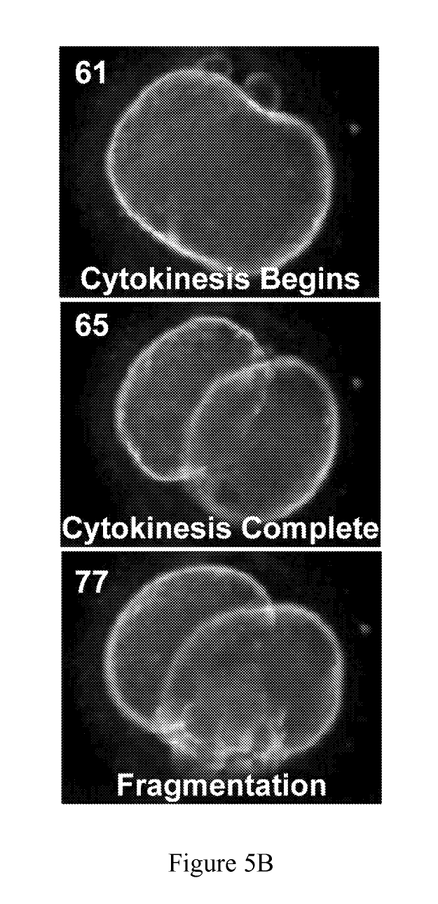

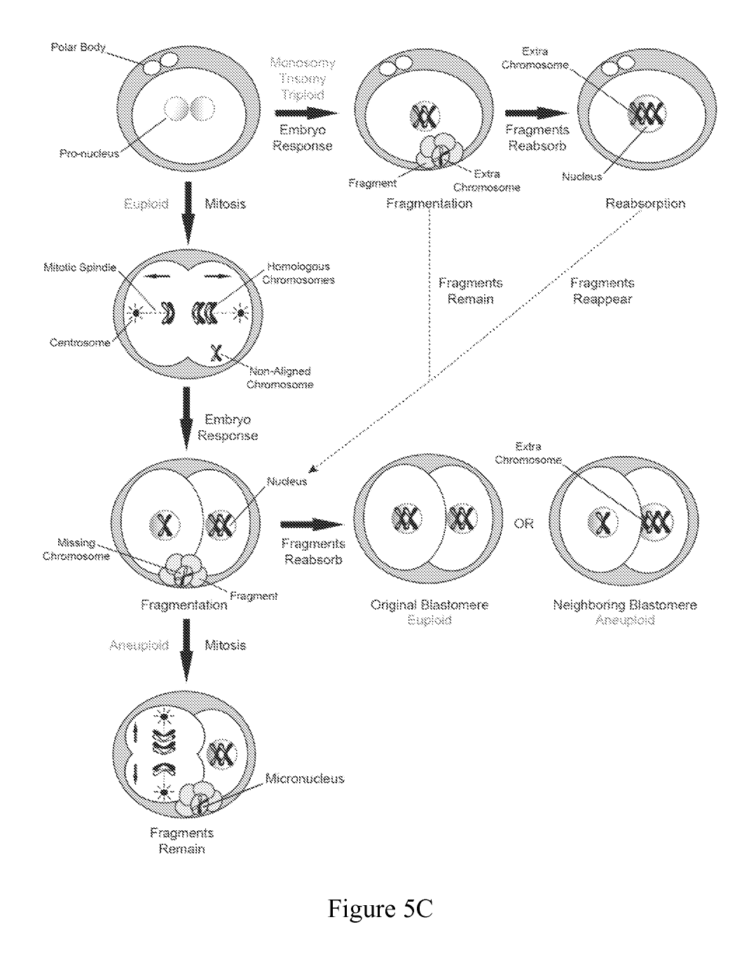

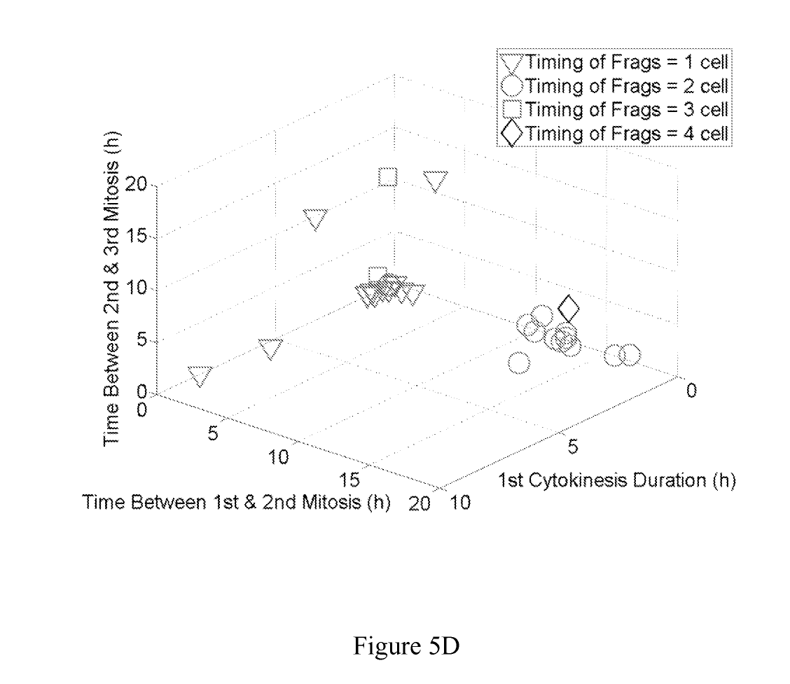

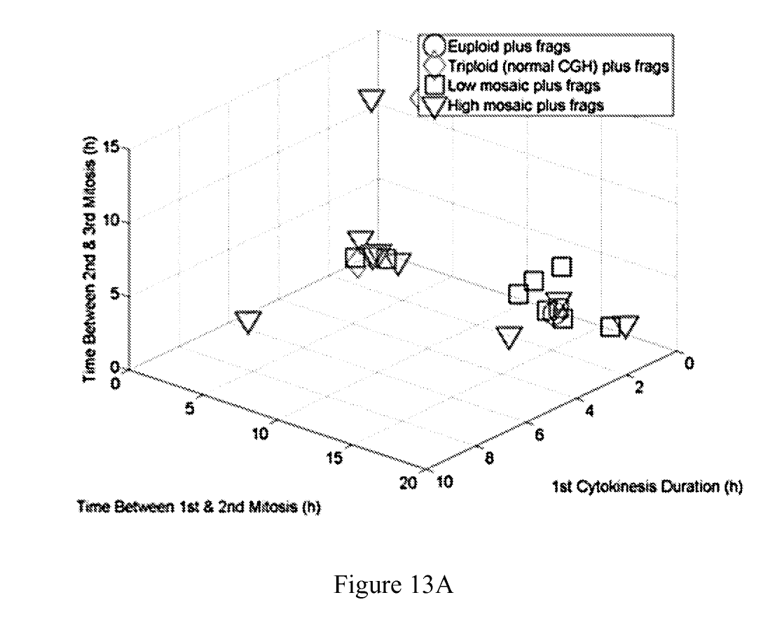

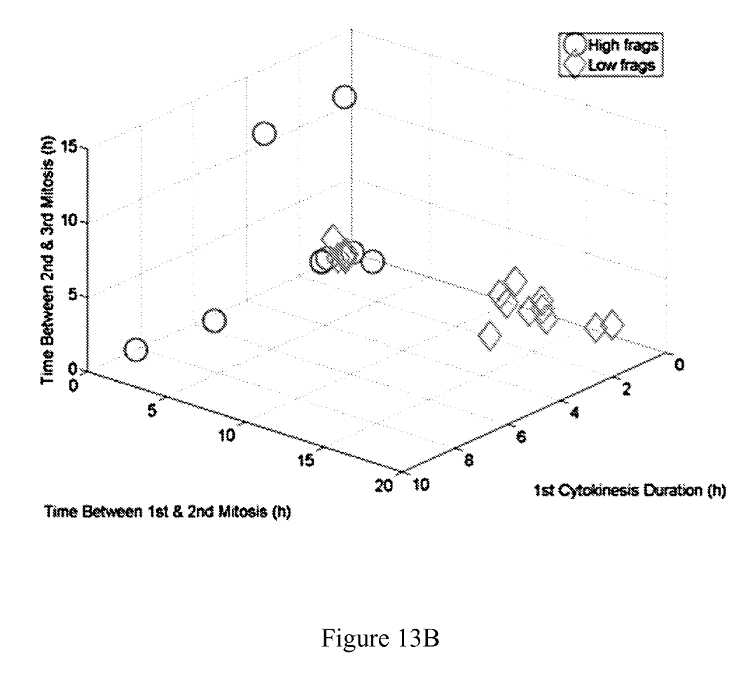

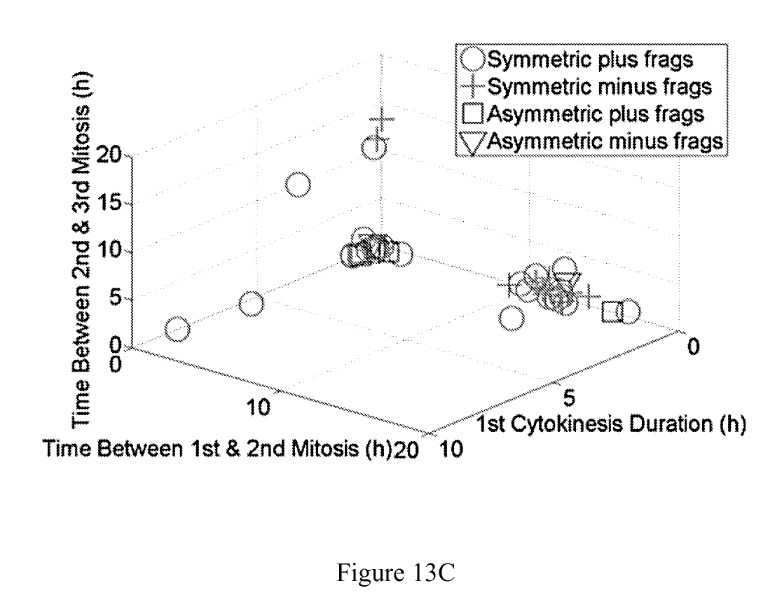

We next focused cell cycle parameter analysis on the embryos that exhibited fragmentation and determined the number of embryos with underlying meiotic and mitotic errors, high and low mosaicism, sub-chromosomal errors and those that appeared to be triploid (FIG. 2D, FIG. 13A and Table 1c). We observed that all of the aneuploid embryos with additional unbalanced sub-chromosomal errors exhibited fragmentation, whereas the one embryo with a balanced translocation between two blastomeres did not (Table 3). Moreover, we observed that additional fragmentation criteria such as the degree and developmental timing of cellular fragmentation or the inclusion of blastomere asymmetry in combination with cell cycle parameter analysis might also aid in embryo assessment (FIG. 5D and FIGS. 13B and 13C). Altogether, our findings suggest that while fragmentation alone is not able to predict developmental potential in embryos, especially when it is assessed at a single time point as is common in IVF clinics, time-lapse imaging in conjunction with dynamic fragmentation screening can detect embryonic aneuploidies of diverse origins.

Containment of Chromosomes within Fragments

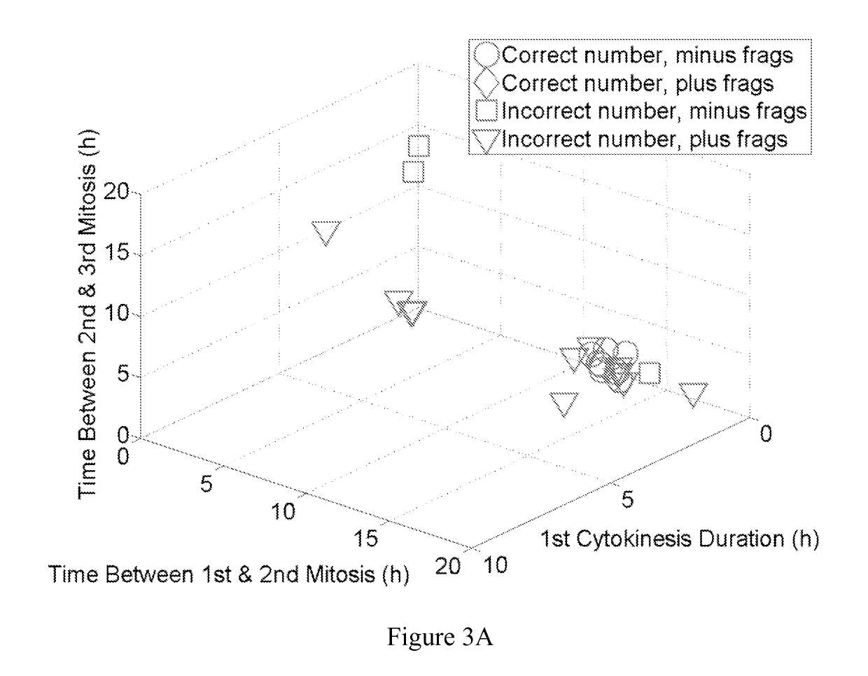

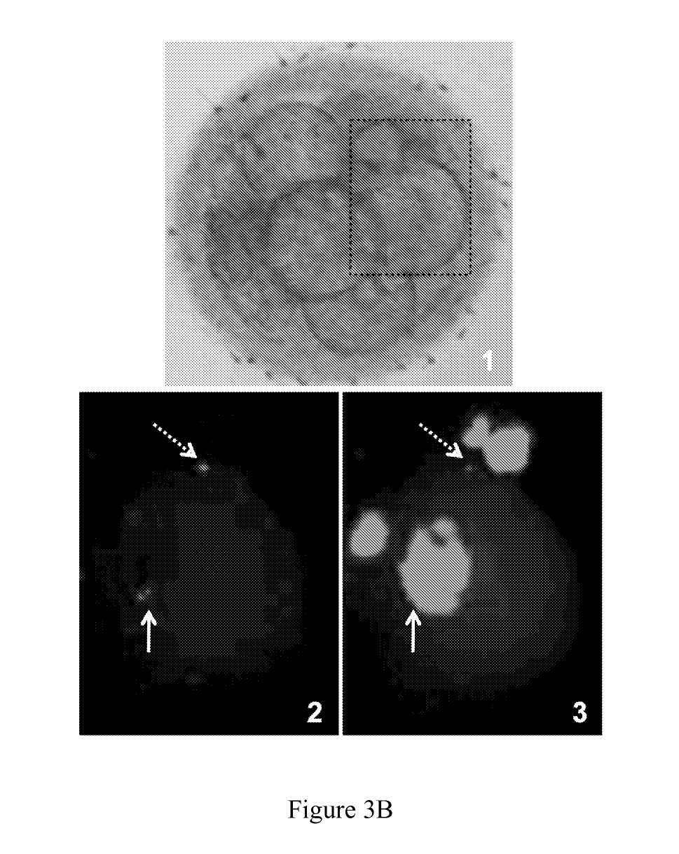

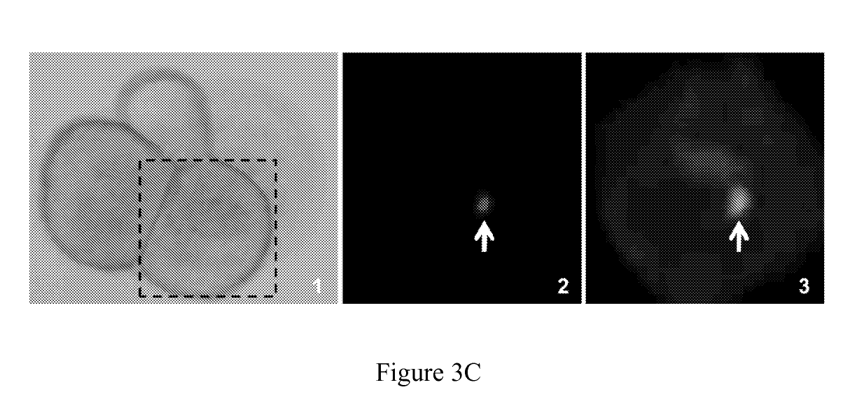

While assessing the chromosome composition of each embryo, we observed that individual blastomeres of aneuploid embryos often exhibited either chromosomal losses or gains, the sum of which did not always add-up to 2 copies of each chromosome per blastomere in each embryo (eg., 8 copies of each autosome in a 4-cell embryo; Table 2). More detailed analysis of chromosome composition (Table 2) also revealed complex genotypes in some embryos that were not in agreement with the typical 3:1 chromosomal ratios detected following mitotic non-disjunction even though A-CGH has been validated repeatedly and probe selection provides redundant coverage of all human chromosomes (Gutierrez-Mateo et al. (2011) Feral. Steril. 95:953-958). Thus, given the observation that aneuploid embryos appeared to be associated with fragmentation (FIG. 2), we explored whether missing chromosomes may have been sequestered into fragments during development. Indeed, we determined that fragmentation was observed in the majority of embryos (11 of 14) that did not have the correct total number of copies of a given chromosome (FIG. 3A). We suspect that the lack of observed fragmentation in the three embryos that displayed the incorrect chromosome copy number may be explained by the previous finding that some fragments may not be easily detected by light microscopy, including time-lapse microscopic imaging, but can be recognized at a higher magnification using alternative optics (Antczak, M. & Van Blerkom, supra).

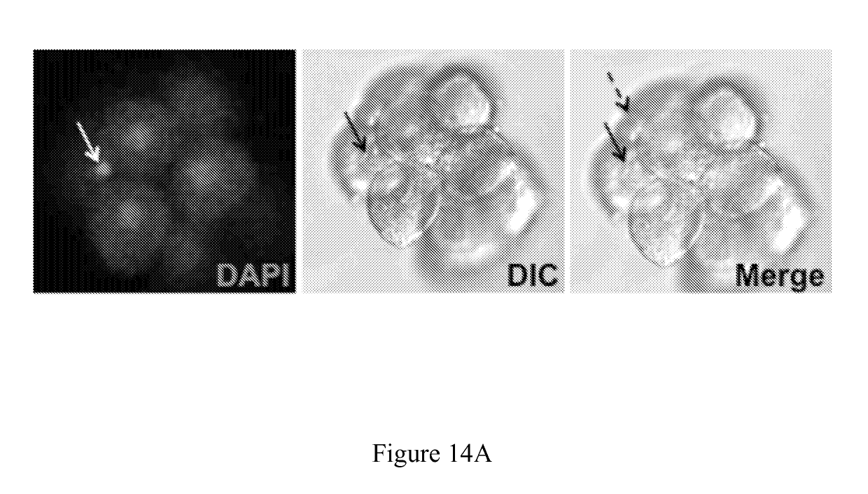

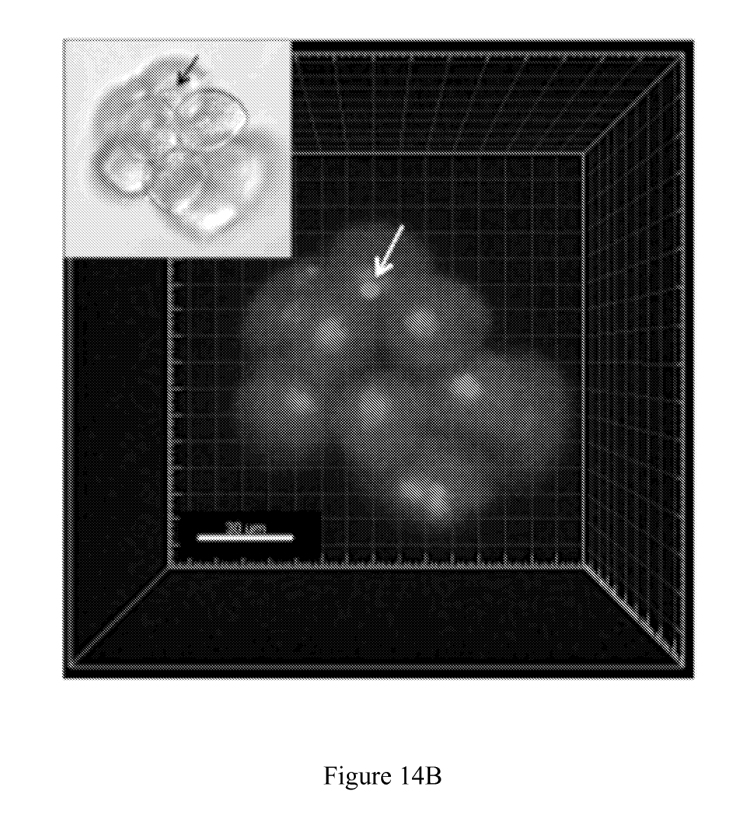

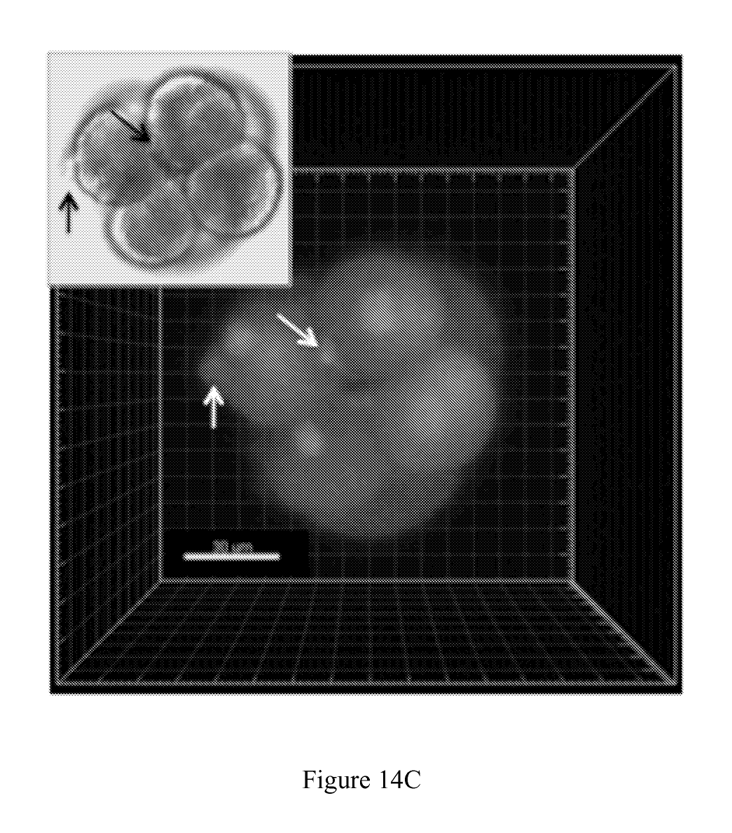

In order to further test whether fragments may contain missing chromosomes, we stained zona pellucida-free cleavage stage human embryos with the nucleic acid dye, 4′,6-diamidino-2-phenylindole (DAPI). As shown by confocal imaging (FIG. 14A) and 3-dimensional modeling (FIGS. 14B and 14C), we observed localization of DNA within fragments (indicated by white arrow in DAPI images and solid black arrow in DIC/merged images). However, we also observed a fragment negative for DAPI staining (shown by dashed black arrow) adjacent to a DAPI-positive fragment, indicating clearly that not all fragments contain nuclear DNA and eliminating the possibility that these fragments were polar bodies, which should have degenerated or initiated degeneration by this stage of development (Munne et al. (1995) Hum. Reprod. 10:1014-1020) and usually contain only a small amount of cytoplasm (FIG. 14A). These findings were supported by DNA-FISH for some of the most commonly affected chromosomes in human embryonic development (Grifo et al. (1996) Curr. Opin. Obstet. Gyn. 8:135-138) in single blastomeres from dissembled embryos as demonstrated in FIGS. 4B and 4C.

Single Blastomere DNA-Fluorescent In Situ Hybridization

To confirm the findings of chromosomal signals distinct from primary nuclei in the blastomeres of intact embryos, we performed DNA-Fluorescent In Situ Hybridization (FISH) on single blastomeres from dissembled embryos with and without fragmentation. While we observed two copies of chromosome 16 in the primary nucleus of a blastomere from a fragmented embryo (indicated by white solid arrow; FIG. 3B), an additional chromosome 16 FISH signal also appeared separate from the primary nucleus (shown by white dashed arrow; FIG. 3B). In another example, we detected 1-2 copies of chromosome 21 (indicated by white solid arrow; FIG. 3C) in a smaller nuclear structure distinct from the primary nucleus of a blastomere taken from an embryo that did not exhibit fragmentation. This data was analogous to the FISH results obtained with non-disassembled embryos and reinforced our findings of nuclear DNA distinct from primary nuclei in human blastomeres.

Chromosome-Containing Fragments May Arise from Micronuclei

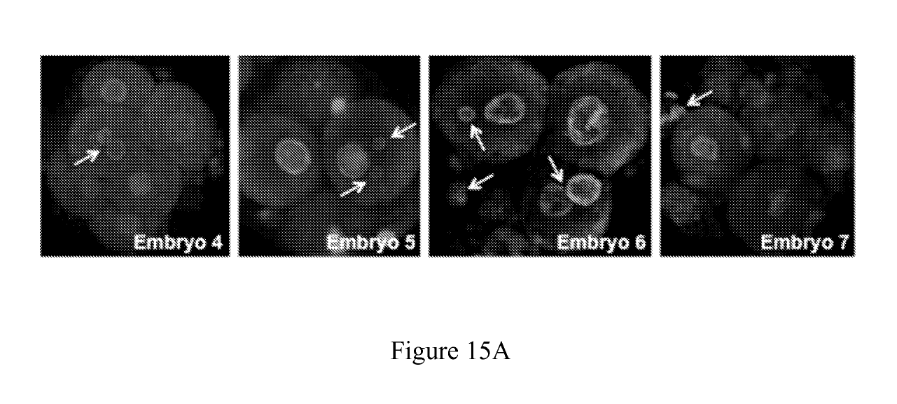

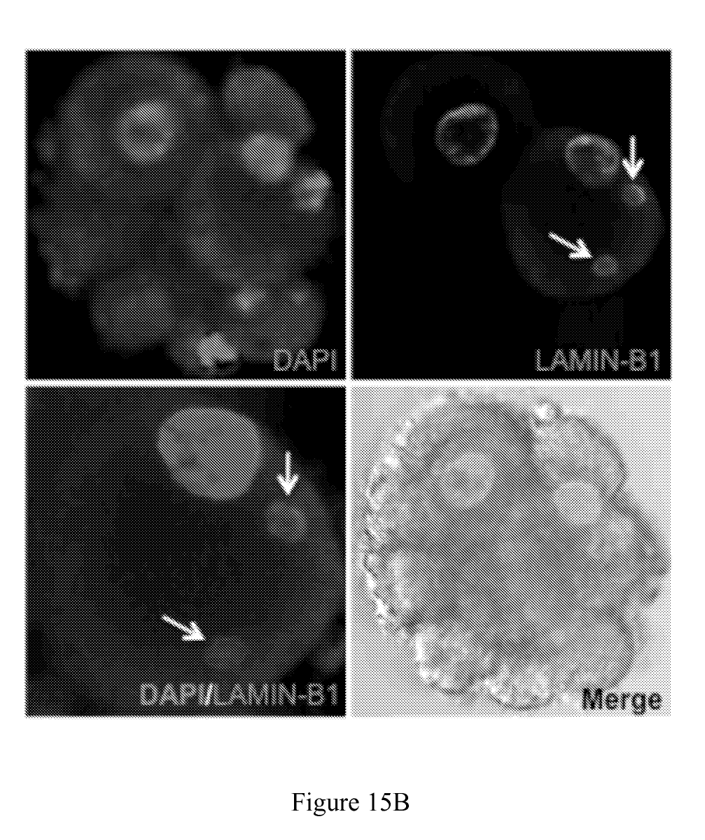

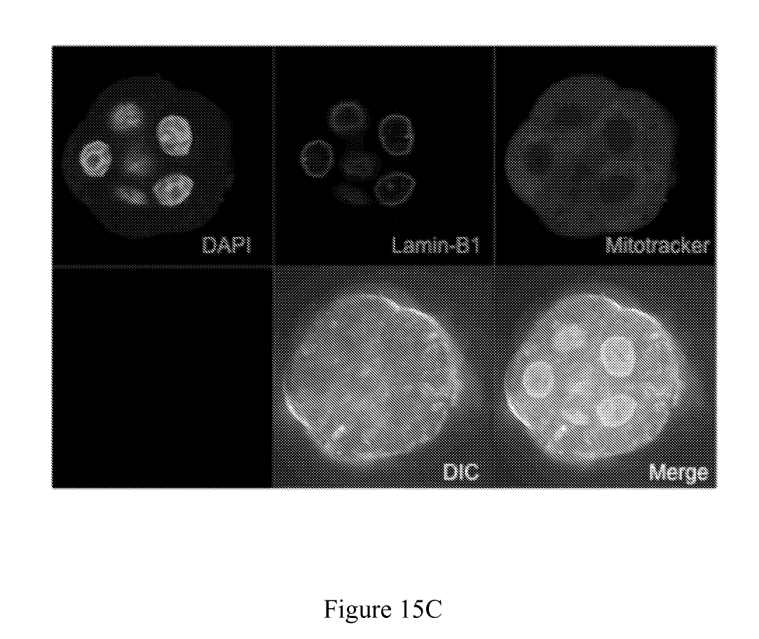

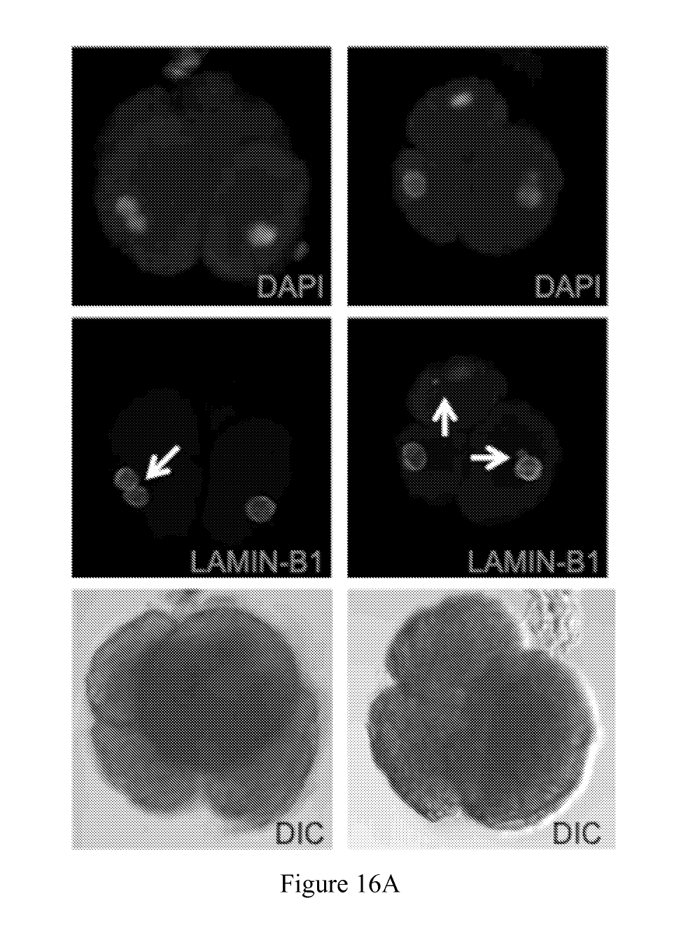

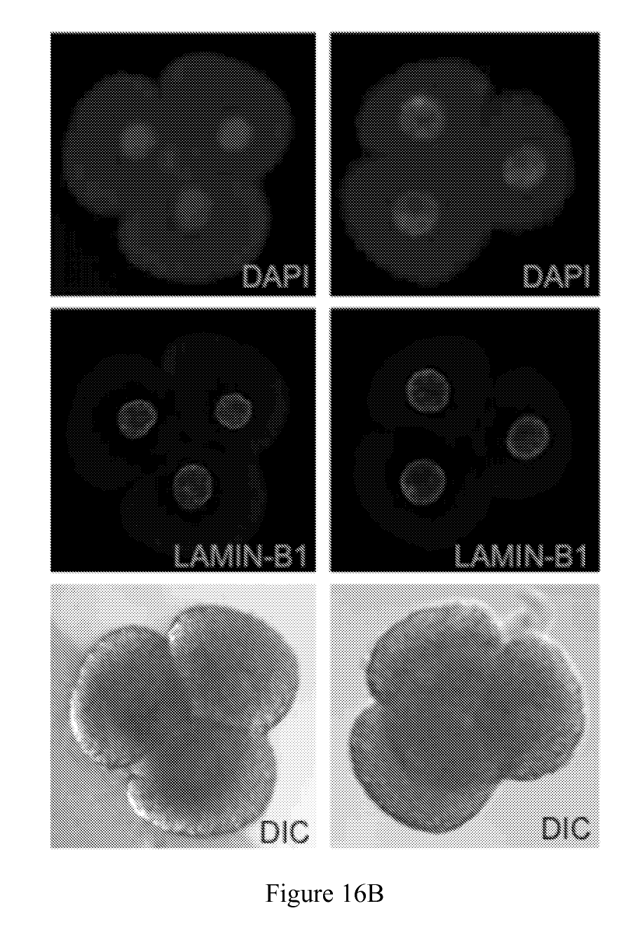

As further evidence of chromosome sequestering within fragments, we immunostained cleavage-stage human embryos with the centromeric marker, centromere protein-A (CENP-A) and the nuclear envelope marker, LAMIN-B1. Not only did we observe positive CENP-A expression in LAMIN-B1 encapsulated nuclei of blastomeres, but we also detected CENP-A immunosignals indicative of multiple missing chromosomes in cellular fragments as well (indicated by white arrows; FIG. 4A and FIGS. 16A-16B). Notably, CENP-A expression was also observed in small structures enveloped by LAMIN-B1 expression (FIG. 4A and FIGS. 15A-15B) that resembled those detected by DNA-FISH (FIG. 3C). We termed these structures embryonic micronuclei and based on their distinct expression pattern, suggest that missing chromosomes are encapsulated in micronuclei prior to being sequestered into fragments (shown by white arrows; FIG. 4A and FIGS. 15A-15B). This was in contrast to our results with mouse embryos, which only exhibited Lamin-B1 expression around the primary nucleus of each blastomere in cleavage-stage embryos (FIG. 4B and FIG. 15C). We also found that encapsulated micronuclei were observed in greater than 50% (13 out of 25) of cleaving human embryos, which is concordant with the percentage of mitotic chromosomal errors detected in this study (FIG. 1A and FIGS. 8A and 8B).

In order to determine whether the appearance of embryonic micronuclei had any effects on developmental potential, we cultured an additional set of human embryos from the zygote to approximately the 4-cell stage and monitored embryonic development by time-lapse imaging. Once the cell cycle parameter values were determined for each embryo, we immunostained for LAMIN-B1; blinded results were then scored for normal and abnormal parameter timing. While LAMIN-B1 expression was confined to the primary nucleus of each blastomere in embryos with normal parameter timing windows, multiple LAMIN-B1 encapsulated micronuclei were detected in one or more embryonic blastomeres of all embryos with abnormal parameter timing (FIGS. 4C, 4D, and 16). When each embryo was graphed as a point in a 3-dimensional parameter plot, the embryos without micronuclei concentrated in a similar three-dimensional area as embryos with normal A-CGH profiles, whereas the embryos with micronuclei exhibited more sporadic parameter clustering (FIG. 4E). Thus, contrary to notions that chromosomal errors are only due to meiotic or mitotic non-disjunction, which manifests as the chromosomes are congressing and then segregating from one another on the spindle (Harrison et al. (2000) Zygote 8, 217-224), our analysis suggests that there is a contribution to aneuploidy that may arise from fragmentation of blastomeres carrying human chromosomes.

Image Analysis of Embryonic Micronuclei in Cleavage-Stage Human Embryos

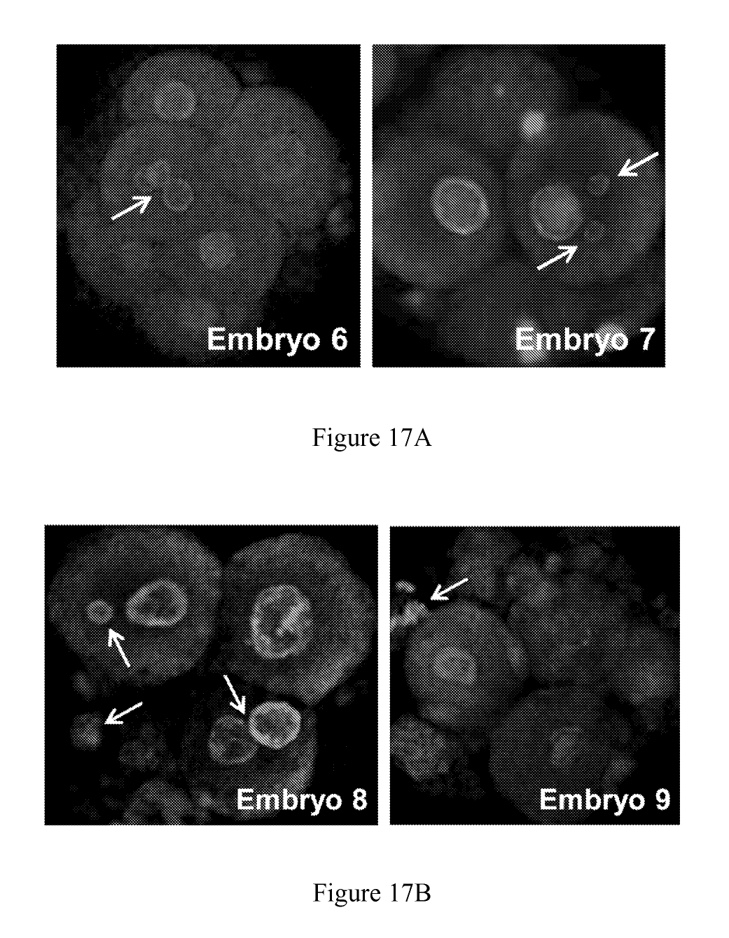

For improved visualization of embryonic micronuclei in the blastomeres and cellular fragments of human embryos, we immunostained additional cleavage-stage human embryos with LAMIN-B1 and CENP-A, but did not perform DIC imaging. As FIGS. 17A demonstrates, we observed multiple micronuclei of varying sizes shown by LAMIN-B1 and DAPI staining (indicated by white arrows) in the blastomeres of embryos that did not exhibit fragmentation. Moreover, we also detected positive CENP-A immunosignals indicative of chromosome sequestering into cellular fragments in embryos with fragmentation (shown by white arrows; FIG. 17B). These results further supported the findings of chromosome sequestering into micronuclei/fragments during human embryo development.

Detection of Embryonic Micronuclei by Brightfield Time-Lapse Imaging

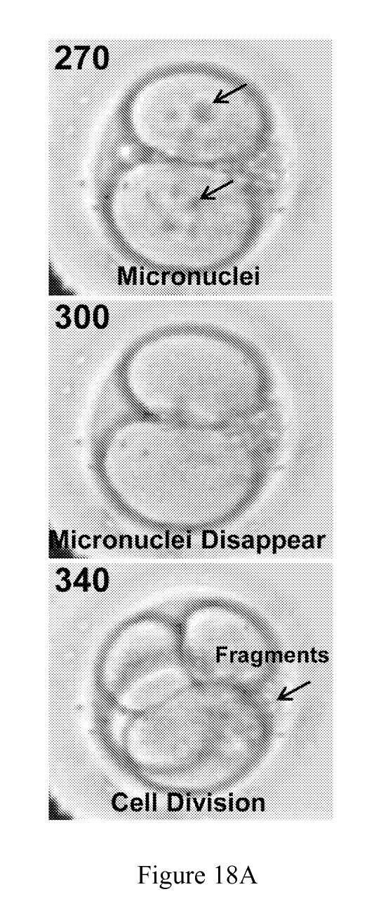

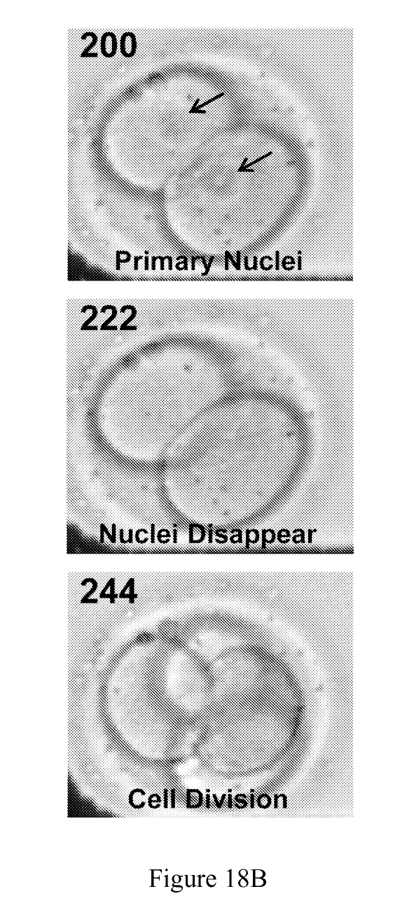

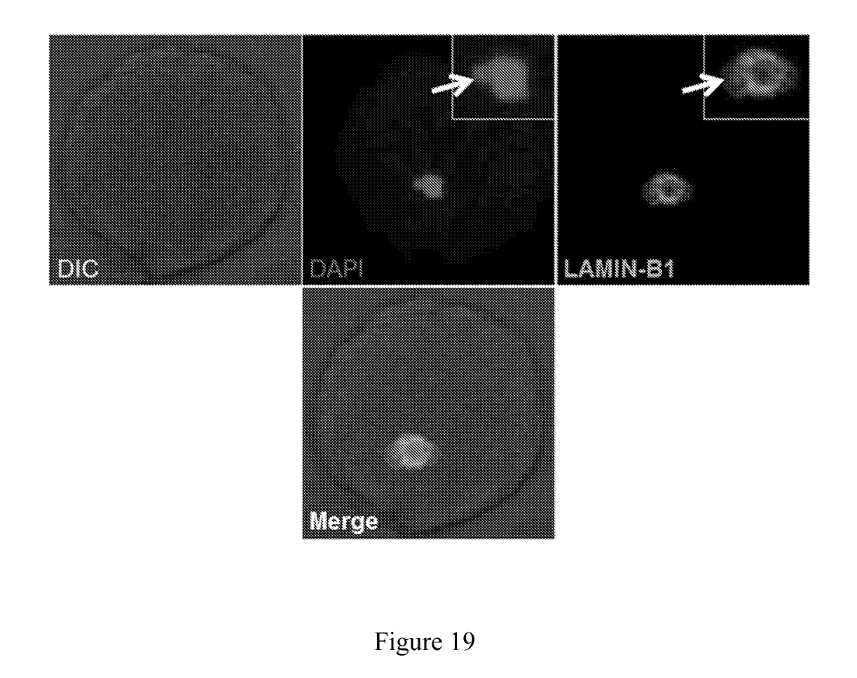

Given that the visualization of embryonic micronuclei in human embryos by LAMIN-B1 immunostaining requires that the embryos be fixed, we explored whether we could non-invasively detect micronuclei by time-lapse imaging as the embryos developed. For this purpose, we utilized brightfield rather than darkfield image analysis to provide the necessary illumination contrast to visualize the nuclear composition within each blastomere. Indeed, we observed the appearance of several dark nuclear structures consistent with micronuclei in the blastomeres of some embryos with fragmentation by brightfield imaging, which simultaneously disappeared prior to cell division (FIG. 18A) in a similar manner to embryos with only intact primary nuclei in their blastomeres that did not exhibit fragmentation (FIG. 18B). This indicates that brightfield image analysis of unstained human embryos can be used to non-invasively detect micronuclei as the embryos develop and assist in the determination of which embryos are aneuploid and less likely to progress in development.

Additional Sub-Chromosomal Losses and Gains in Aneuploid Embryos

Besides detecting whole chromosomal losses and gains in several human embryos, we also observed additional sub-chromosomal errors in 10 of the aneuploid embryos (Table 6). This was in sharp contrast to euploid embryos, which did not exhibit either whole or partial losses and gains. Interestingly, all of the aneuploid embryos with unbalanced sub-chromosomal translocations (FIG. 11B) also exhibited fragmentation, whereas the one embryo with a balanced translocation between two blastomeres (FIG. 11A) did not have fragmentation (Table 6). These findings suggested that portions of chromosomes may also be sequestered into embryonic micronuclei and lost to cellular fragments during development. Furthermore, we also observed that the generation of partial chromosomal gains and losses was restricted primarily to embryos with mitotic errors (Table 6). When we measured the cell cycle parameters in these embryos we determined that the majority of the embryos exhibited abnormal parameter timing, particularly those that appeared to be triploid by image analysis and embryos with high mitotic mosaicism (FIG. 11C). Altogether, this suggests that there is a relationship between sub-chromosomal instability and aneuploidy in the human embryo, which may be reflected in the incidence of cellular fragmentation and cell cycle parameter timing.

Proposed Model of Aneuploidy Generation in Human Embryos

Our time-lapse image analysis suggests that as development proceeds, embryonic fragments may remain as separate units that carry chromosomal DNA and cytoplasm, or alternatively, fragments may also be reabsorbed by the same blastomere from which they were produced or fuse with a neighboring blastomere(s) (FIG. 5A) as previously suggested (Van Blerkom et al. (2001) Hum. Reprod. 16:719-729; Hardarson et al. (2002) Reprod. Biomed. Online 5, 36-38; Lemmen et al. (2008) Reprod. Biomed. Online 17:385-391). If a chromosome-containing fragment fuses with the blastomere from which it originated, it could potentially restore embryonic euploidy status following nuclear envelope breakdown (FIG. 5C), which may explain the single euploid embryo with fragmentation observed in this study (FIG. 2B) and also previous findings of occasional chromosomal correction during embryo development (Barbash-Hazan et al. (2009) Fertil. Steril. 92:890-896; Munne et al. (2005) Fertil. Steril. 84, 1328-1334). However, equally likely is the fusion of a fragment with sequestered chromosome(s) to a neighboring blastomere (FIG. 5C), resulting in the complex genotypes observed in this study (Table 2) and others (Vanneste et al., supra; Johnson et al., supra)

After further evaluating the correlation between the timing of fragmentation and the cell cycle imaging parameters, we observed that the most significant effects on parameter windows were observed in embryos in which fragmentation occurred either at the 1-cell stage or to a lesser extent at the 3 to 4-cell stage (FIG. 5D). Following analysis of embryonic chromosomal composition, we determined that embryos with meiotic errors or those that appeared to be triploid by imaging consistently exhibited fragmentation at the 1-cell stage prior to the first cytokinesis (Table 1c). In contrast, fragmentation was typically observed later, following the division of 1-cell to 2-cells in embryos with mitotic errors (Table 1c) and also often during interphase, or following the completion of the first cytokinesis (FIG. 5B). It is important to note that the blastomeres of the embryos with mitotic errors, which began fragmenting at the 1-cell stage (Table 1c), also exhibited complex mitotic mosaicism with multiple missing chromosomes, indicating that these embryos may have inherited a meiotic error followed by generation of a mitotic error. For those embryos with mitotic errors, we also propose in our model that these embryos likely divided before all the chromosomes had a chance to properly align on the mitotic spindle (FIG. 5C), which is supported by findings of lagging chromosomes during anaphase in human embryos (Coonen et al. (2004) Hum. Reprod. 19:316-324). Whether the generation of micronuclei and cellular fragmentation represents a potential means to correct the embryonic aneuploidy (FIG. 5C) or is the initiation of eventual demise later in development (Hardy et al. (2001) Proc. Natl. Acad. Sci. 98, 1655-1660; Spanos et al. (2002) Reproduction 124:353-363) remains to be determined. The generation of aneuploidy in human embryos, as defined here, precedes the major wave of embryonic genome activation that occurs at approximately the 8-cell stage (Wong et al. (2010) Nature Biotechnol. 28:1115-1121; Dobson et al. (2004) Hum. Mol. Genet. 13:1461-1470; Zhang et al. (2009) PLoS One 4:e7844; Galan et al. (2010) PLoS ONE 5:e13615; Vanneste et al. (2009) Nature Med. 15:577-583; Veeck et al. (1993) Fertil. Steril. 59:1202-1207; Miller et al. (1995) Obst. Gynecol. 85:999-1002). Thus, the human embryo has limited ability to respond with a transcriptional cascade that could culminate with apoptosis, but must rely on translational programs.

Discussion

Over the last few decades, elegant studies in model systems including yeast, flies, worms, fish and frogs have compared chromosome dynamics in wildtype and mutant cells that display diverse phenotypes (Lieb et al. (1998) Cell 92:265-277; Stear & Roth (2002) Genes Dev. 16:1498-1508; Hartwell & Smith (1985) Genetics 110:381-395; Hartwell et al. (1974) Science 183:46-51; Albertson et al. (1984) Dev. Biol. 101:61-72; Gonczy et al. (1999) J. Cell Biol. 144:927-946; Abrams et al. (2012) Cell In Press; Montag et al. (1988) Chromosoma 96:187-196). Particularly in Caenorhabditis elegans, studies of mutations in genes that affect the first embryonic cell divisions are enlightening. For example, in 1994 Gonczy and colleagues reported a collection of 48 maternal-effect embryonic lethal mutations on chromosome III that they characterized phenotypically by time-lapse DIC video microscopy (Gonczy et al., supra). The mutations mapped to 34 loci and were characterized by defects in pronuclear migration, rotation of centrosomes and associated pronuclei, spindle assembly, chromosome segregation, anaphase spindle positioning and cytokinesis. Subsequent studies have examined the molecular mechanisms that underlie these phenotypic classes and others (Lieb et al., supra; Stear & Roth, supra; Fox et al. (2010) Genes Dev. 24:2294-2302; Mendiburo et al. (2011) Science 334:686-690).

In other species such as the zebrafish, Danio Rerio and the frog, Xenopus laevis, karyomeres form to accommodate the large cells formed following fertilization (Abrams et al., supra; Montag et al., supra). Karyomeres are thought to provide mitotic intermediates that are comprised of chromatin masses surrounded by nuclear envelope, which then fuse to form a single nucleus as recently described in more detail by Mullins and colleagues (Abrams et al., supra). When we compare these studies to our data, we observe that a subset of images from these organisms clearly resemble those we observed with formation of multiple micronuclei in human embryos. This suggests the possibility that embryonic micronuclei observed in human embryos may be formed via conserved pathways that allow encapsulation of chromosomes in nuclear envelope either under normal developmental conditions or in response to chromosome detachment from the spindle during cleavage divisions.

In this study, we observed that chromosomally normal embryos display strict and tightly-clustered cell cycle parameters, whereas chromosomally abnormal embryos exhibit more diverse parameters that may or may not overlap those of euploid embryos. By the 4-cell stage of development, we observed that dynamic assessment of cell cycle parameters in conjunction with fragmentation analysis and blastomere asymmetry assists in the differentiation between the type of error (meiotic vs. mitotic), detects chromosomal duplications (trisomies) and deletions (monosomies) and provides a reliable readout of the degree of mitotic mosaicism (high vs. low) in human embryos. Furthermore, we observed that the generation of partial chromosomal gains and losses was restricted primarily to embryos with mitotic errors, suggesting a relationship between sub-chromosomal instability and aneuploidy in the human embryo. We also demonstrate that human embryonic aneuploidy and mosaicism of chromosome content between blastomeres (Vanneste, supra; Johnson, supra; Kuo, supra; Baart et al., supra) may have contributions from a phenomenon that encompasses the formation of embryonic micronuclei, cellular fragmentation and resorption. Thus, practices that were once promoted such as embryo surgery for the removal of fragments are likely to result in the removal of micronuclei and perhaps genetic information and may potentially be deleterious (Keltz et al. (2006) Feral. Steril. 86:321-324; Eftekhari-Yazdi et al. (2006) Reprod. Biomed. Online 13:823-832).

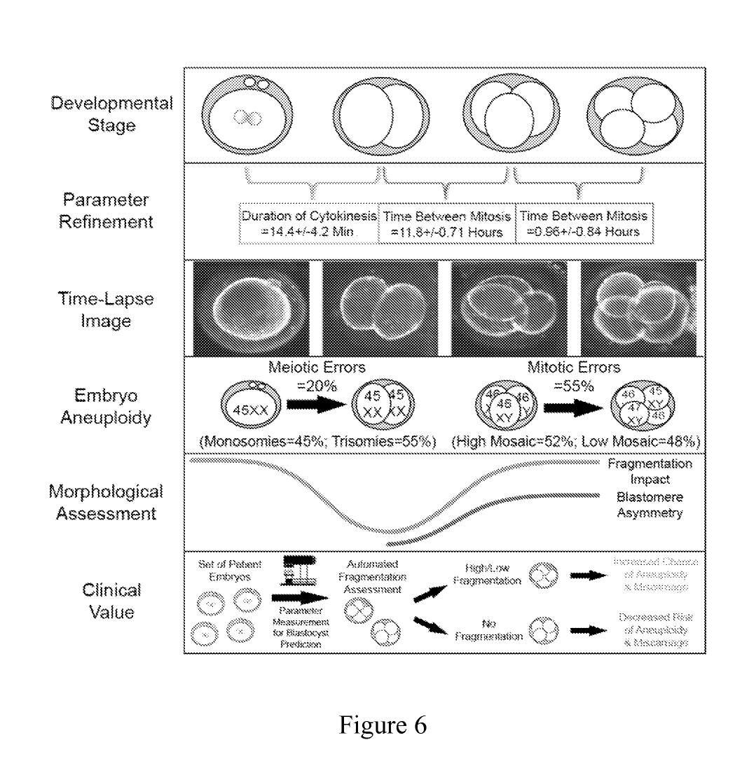

Based on the timing of fragmentation and our time-lapse image analysis, we suggest that the human embryo may initially undergo fragmentation, rather than cell death, in response to aneuploidy. We do not currently understand the mechanisms underlying the increased aneuploidy rates and frequent fragmentation observed in cleaving human embryos. However, we relate our data here (FIG. 6) to that in other organisms where genetic instability may be associated with the unique epigenetic programs of pre-implantation development (Monk et al. (1987) Development 99:371-382; Howlett & Reik (1991) Development 113:119-127; Sanford et al. (1987) Genes Dev. 1:1039-1046). Alternatively, the generation of human embryonic aneuploidy may be related to the paternal contribution of the centrosome (and other spindle components) by the sperm for the first mitotic division (Sathananthan et al. (1991) Proc. Natl. Acad. Sci. 88:4806-4810; Palermo et al. (1994) Hum. Reprod. 9:1220-1225). Regardless of mechanism, it is likely that non-invasive assessment of development of human embryos as previously described (Wong et al, supra) and further confirmed in retrospective studies of clinical embryos will assist in the prediction of embryo viability (Meseguer et al. (2011) Hum. Reprod. 26:2658-2671). Moreover, distinction between euploid and aneuploid embryos prior to transfer may contribute to improvements in IVF outcomes by potentially reducing the inadvertent transfer of embryos that would most-likely result in embryonic lethality and spontaneous miscarriage.

TABLE 1
Distinction between euploid, aneuploid and triploid embryos by A-CGH, cell cycle parameter analysis and dynamic
fragmentation assessment. (a) A table showing the number and percentage of human embryos used in this study, which developed
beyond the 1-cell stage and were categorized into the different ploidy groups (euploid, aneuploid and triploid) based on the results of
the A-CGH and time-lapse image analysis of individual blastomeres. (b) Comparison of the individual parameter values and standard
deviations between embryos with normal CGH profiles, meiotic and mitotic errors and embryos with high or low mitotic mosaicism.
(c) A table depicting the correlation between the number and percentage of euploid, aneuploid or triploid human embryos and the
incidence and timing of cellular fragmentation.
a

| | | | | | | Aneuploid | | | | |
|---|---|---|---|---|---|---|---|---|---|---|
| | Progressed | | | | | (Contains | | | | |
| | Beyond | Conclusive | Euploid | Aneuploid | Triploid | No | Meiotic | Mitotic | Complex | Simple |
| | 1-Cell | A-CGH | (Normal | (Abnormal | (Normal | Euploid | Chromosomal | Chromosomal | Mitotic | Mitotic |
| | Stage | Data | A-CGH) | A-CGH) | A-CGH) | Blastomeres) | Errors | Errors | Mosaicism | Mosaicism |
| Number of | 53 | 45 | 8 | 34 | 3 | 29 | 9 | 25 | 20 | 5 |
| Embryos | | | | | | | | | | |
| Total | 75 | 53 | 45 | 45 | 45 | 34 | 45 | 45 | 25 | 25 |
| Number | | | | | | | | | | |
| of Embryos | | | | | | | | | | |
| Percentage | 70.7% | 84.9% | 17.8% | 75.5% | 6.7% | 85.3% | 20% | 55% | 80% | 20% |

Note the high percentage of aneuploid embryos that did not contain a single euploid blastomere capable of contributing to the embryo
proper later in development as well as the number and percentage of embryos that acquired meiotic or mitotic errors and those with
individual blastomeres characterized by complex or simple mitotic mosaicism in this study.

b

| | | | | High Mosaic | Low Mosaic |
|---|---|---|---|---|---|
| Parameter | Normal CGH | Meiotic Error | Mitotic Error | Mitotic Error | Mitotic Error |
| Measurements | (N = 8) | (N = 9) | (N = 25) | (N = 13) | (N = 12) |
| Duration of first | 14.4 ± 4.2 Min. | 117.2 ± 166.5 Min. | 36.0 ± 66.9 Min. | 52.7 ± 89.8 Min. | 17.9 ± 16.8 Min. |
| cytokinesis | | | | | |
| Time between | 11.8 ± 0.71 Hours | 4.0 ± 5.2 Hours* | 6.4 ± 6.6 Hours* | 3.5 ± 6.2 Hours** | 9.6 ± 5.6 Hours |
| first and second | | | | | |
| mitosis | | | | | |
| Time between | 0.96 ± 0.84 Hours | 2.0 ± 4.3 Hours | 2.0 ± 3.9 Hours | 2.2 ± 4.1 Hours | 1.8 ± 3.8 Hours |
| second and third | | | | | |
| mitosis | | | | | |

The mean values and standard deviations of embryos that were determined to be chromosomally normal by A-CGH can be
used to potentially refine the parameters predictive of blastocyst formation; * = p < 0.05; ** = p < 0.01

c

| | | | | | | | | | | | Mitotic |
|---|---|---|---|---|---|---|---|---|---|---|---|
| | | | | | | | Meiotic | | | | Error |
| | | | | Meiotic | Meiotic | Meiotic | Error | Mitotic | Mitotic | | Begin |
| | Euploid | Aneuploid | Triploid | Error | Error | Error | Begin | Error | Error | Mitotic Error | Frag- |
| | With | With | With | With | Without | Begin | Fragmenting | With | Without | Begin | menting |
| | Cellular | Cellular | Cellular | Cellular | Cellular | Fragmenting | 2-4 Cell | Cellular | Cellular | Fragmenting | 2-4 Cell |
| | Fragments | Fragments | Fragments | Fragments | Fragments | 1-Cell Stage | Stage | Fragments | Fragments | 1-Cell Stage | Stage |
| Number of | 1 | 28 | 3 | 9 | 0 | 8 | 1 | 19 | 6 | 5^{#} | 14 |
| Embryos | | | | | | | | | | | |
| Total | 8 | 34 | 3 | 9 | 9 | 9 | 9 | 25 | 25 | 19 | 19 |
| Number | | | | | | | | | | | |
| of Embryos | | | | | | | | | | | |
| Percentage | 12.5% | 82.4% | 100% | 100% | 0% | 89% | 11% | 76% | 24% | 26% | 74% |

^{#}Note that the 5 embryos, which begin fragmenting at the 1-cell stage, were also characterized by complex mitotic mosaicism, suggesting
that these embryos likely acquired a meiotic error followed by a mitotic error.

TABLE 2
Comparison of chromosome composition in aneuploid embryos with simple and complex mitotic errors. Full
chromosome analysis of a (a) fragmented and (b) non-fragmented 4-cell embryo pictured in FIG. 2a1 and 2, respectively,
showing that the aneuploid embryo with fragmentation is missing 2 copies of chromosome 6 from two of its blastomeres.
(c)-(e). The chromosomal status of all blastomeres in additional embryos with mitotic errors that exhibit chromosomal
losses or gains as well as atypical chromosomal ratios that are inconsistent with mitotic non-disjunction. Note that only
2 chromosomes are unaffected in the blastomeres of the embryo depicted in e, which is likely to have incurred both
meiotic and mitotic errors.

| | | | | | | | | | | | | | | | | | | | | | | | |
|---|---|---|---|---|---|---|---|---|---|---|---|---|---|---|---|---|---|---|---|---|---|---|---|
| Ch. | 1 | 2 | 3 | 4 | 5 | 6 | 7 | 8 | 9 | 10 | 11 | 12 | 13 | 14 | 15 | 16 | 17 | 18 | 19 | 20 | 21 | 22 | Sex |

a

| | | | | | | | | | | | | | | | | | | | | | | | |
|---|---|---|---|---|---|---|---|---|---|---|---|---|---|---|---|---|---|---|---|---|---|---|---|
| B1 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | XX |
| B2 | 2 | 2 | 2 | 2 | 2 | 1 | 2 | 2 | 2 | 2 | 2 | 2 | 2 | 2 | 2 | 2 | 2 | 2 | 2 | 2 | 2 | 2 | XX |
| B3 | 2 | 2 | 2 | 2 | 2 | 1 | 2 | 2 | 2 | 2 | 2 | 2 | 2 | 2 | 2 | 2 | 2 | 2 | 2 | 2 | 2 | 2 | XX |
| B4 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | XX |
| Sum | 8 | 8 | 8 | 8 | 8 | 6 | 8 | 8 | 8 | 8 | 8 | 8 | 8 | 8 | 8 | 8 | 8 | 8 | 8 | 8 | 8 | 8 | 8 |

b

| | | | | | | | | | | | | | | | | | | | | | | | |
|---|---|---|---|---|---|---|---|---|---|---|---|---|---|---|---|---|---|---|---|---|---|---|---|
| B1 | 2 | 2 | 2 | 2 | 2 | 2 | 2 | 2 | 2 | 2 | 2 | 2 | 2 | 2 | 2 | 2 | 2 | 2 | 2 | 2 | 3 | 2 | XX |
| B2 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | XX |
| B3 | 2 | 2 | 2 | 2 | 2 | 2 | 2 | 2 | 2 | 2 | 2 | 2 | 2 | 2 | 2 | 2 | 2 | 2 | 2 | 2 | 2 | 2 | XX |
| B4 | 2 | 2 | 2 | 2 | 2 | 2 | 2 | 2 | 2 | 2 | 2 | 2 | 2 | 2 | 2 | 2 | 2 | 2 | 2 | 2 | 1 | 2 | XX |
| Sum | 8 | 8 | 8 | 8 | 8 | 8 | 8 | 8 | 8 | 8 | 8 | 8 | 8 | 8 | 8 | 8 | 8 | 8 | 8 | 8 | 8 | 8 | 8 |

c

| | | | | | | | | | | | | | | | | | | | | | | | |
|---|---|---|---|---|---|---|---|---|---|---|---|---|---|---|---|---|---|---|---|---|---|---|---|
| B1 | 2 | 2 | 2 | 2 | 1 | 2 | 2 | 2 | 2 | 2 | 2 | 2 | 2 | 2 | 2 | 2 | 2 | 2 | 2 | 2 | 2 | 2 | XY |
| B2 | 2 | 2 | 1 | 2 | 1 | 2 | 2 | 2 | 2 | 2 | 2 | 2 | 2 | 2 | 1 | 1 | 2 | 2 | 2 | 2 | 2 | 2 | XY |
| B3 | 2 | 2 | 2 | 2 | 3 | 2 | 2 | 2 | 2 | 2 | 2 | 2 | 2 | 2 | 2 | 2 | 2 | 2 | 2 | 2 | 2 | 2 | XY |
| B4 | 2 | 2 | 2 | 2 | 2 | 2 | 2 | 2 | 2 | 2 | 2 | 2 | 2 | 2 | 3 | 3 | 2 | 2 | 2 | 2 | 2 | 2 | XY |
| Sum | 8 | 8 | 7 | 8 | 7 | 6 | 8 | 8 | 8 | 8 | 8 | 8 | 8 | 8 | 8 | 8 | 8 | 8 | 8 | 8 | 8 | 8 | 8 |

d

| | | | | | | | | | | | | | | | | | | | | | | | |
|---|---|---|---|---|---|---|---|---|---|---|---|---|---|---|---|---|---|---|---|---|---|---|---|
| B1 | 1 | 2 | 2 | 2 | 2 | 2 | 2 | 2 | 2 | 1 | 2 | 2 | 2 | 2 | 2 | 1 | 2 | 2 | 2 | 2 | 2 | 2 | XX |
| B2 | 3 | 2 | 2 | 2 | 2 | 2 | 2 | 2 | 2 | 1 | 2 | 2 | 2 | 2 | 2 | 1 | 2 | 2 | 2 | 2 | 2 | 2 | XX |
| B3 | 1 | 2 | 2 | 2 | 2 | 2 | 2 | 2 | 2 | 1 | 2 | 2 | 2 | 2 | 2 | 1 | 2 | 2 | 2 | 2 | 2 | 2 | XX |
| B4 | 1 | 2 | 2 | 2 | 2 | 2 | 2 | 2 | 2 | 3 | 2 | 2 | 2 | 2 | 2 | 3 | 2 | 2 | 2 | 2 | 2 | 2 | XX |
| Sum | 6 | 8 | 8 | 8 | 8 | 8 | 8 | 8 | 8 | 6 | 8 | 8 | 8 | 8 | 8 | 6 | 8 | 8 | 8 | 8 | 8 | 8 | 8 |

e

| | | | | | | | | | | | | | | | | | | | | | | | |
|---|---|---|---|---|---|---|---|---|---|---|---|---|---|---|---|---|---|---|---|---|---|---|---|
| B1 | 3 | 1 | 3 | 1 | 2 | 2 | 2 | 2 | 1 | 2 | 2 | 2 | 3 | 2 | 2 | 2 | 2 | 3 | 2 | 3 | 2 | 1 | X |
| B2 | 2 | 3 | 2 | 2 | 2 | 3 | 1 | 1 | 2 | 1 | 2 | 2 | 2 | 2 | 2 | 2 | 2 | 1 | 2 | 2 | 2 | 2 | XX |
| B3 | 2 | 2 | 1 | 1 | 1 | 3 | 2 | 2 | 2 | 2 | 2 | 2 | 2 | 2 | 2 | 2 | 1 | 3 | 2 | 1 | 2 | 2 | XX |
| B4 | 1 | 1 | 2 | 3 | 2 | 2 | 2 | 2 | 2 | 2 | 1 | 3 | 2 | 2 | 3 | 3 | 3 | 1 | 2 | 3 | 1 | 2 | XX |
| B5 | 2 | 1 | 3 | 2 | 2 | 2 | 2 | 2 | 1 | 2 | 2 | 2 | 3 | 2 | 2 | 1 | 2 | 3 | 2 | 3 | 2 | 2 | X |
| B6 | 2 | 2 | 1 | 2 | 2 | 2 | 2 | 2 | 2 | 2 | 2 | 2 | 2 | 2 | 2 | 2 | 1 | 3 | 2 | 1 | 2 | 2 | XX |
| Sum | 12 | 10 | 12 | 11 | 11 | 14 | 11 | 11 | 10 | 11 | 11 | 13 | 14 | 12 | 13 | 12 | 11 | 14 | 12 | 13 | 11 | 11 | 10 |

TABLE 3
Several aneuploid embryos also exhibit sub-chromosomal losses
and gains. A table depicting the sub-chromosomal analysis of certain
low/high mitotic mosaic and triploid embryos with cell cycle parameter
timing illustrated in FIG. 10. Note that one of these embryos had a
balanced translocation between blastomeres and it is the only
embryo that did not exhibit cellular fragmentation.

| Embryo | Aneuploidy | | Fragmentation |
|---|---|---|---|
| Number | Type | Sub-Chromosomal Analysis | Analysis |
| 1 | Low mitotic | Unbalanced partial gain Ch. 10p | Yes |
| | mosaicism | | |
| 2 | Low mitotic | Balanced partial loss/gain Ch. 1q | No |
| | mosaicism | | |
| 3 | High mitotic | Unbalanced partial loss Ch. 10q | Yes |
| | mosaicism | | |
| 4 | Low mitotic | Unbalanced partial loss | Yes |
| | mosaicsim | Ch.1q, 10q and 16q and | |
| | | partial gain Ch.9q | |
| 5 | High mitotic | Unbalanced partial loss Ch.8p, | Yes |
| | mosaicism | 1q and 12q | |
| 6 | High mitotic | Unbalanced partial loss Ch.9q | Yes |
| | mosaicism | and partial gain Ch.1q, 7p, | |
| | | 10q and 16q | |
| 7 | Low mitotic | Unbalanced partial gain Ch. 11q | Yes |
| | mosaicsim | | |
| 8 | Low mitotic | Unbalanced partial loss Ch. 6q | Yes |
| | mosaicism | and 7p | |
| 9 | Triploid | Unbalanced partial gain Ch. 22q | Yes |
| 10 | Triploid | Unbalanced partial gain Ch. 19p | Yes |

TABLE 4
Calculation of embryonic euploidy versus aneuploidy risk. The table
shows the calculation (number of euploid embryos/total number of
embryos), the resulting probability of embryonic euploidy expressed
as a percentage and the supporting literature reference for each
morphological and/or parameter assessment illustrated in FIG. 11.
Note that the highest percentage of embryonic euploidy would be
obtained with the combination of cell cycle parameters that predict
normal CGH and high/low fragmentation analysis.

| | Calcula- | Percentage | |
|---|---|---|---|
| | tion (# | (proba- | |
| Measurement | of euploid/ | bility | |
| (morphological and/or | total # | of embryo | |
| parameter analysis) | of embryos) | euploidy) | Literature Reference |
| No morphological or | 8/45 | 17.8% | Present study and |
| parameter screening | | | Vanneste et al. 2009 |
| Fragmentation | 12/45 | 26.7% | Present study and |
| (high degree) | | | Antczak & Van |
| | | | Blerkom, 1999 |
| Fragmentation | 27/45 | 60% | Present study and |
| (high & low | | | Antczak & Van |
| degree) | | | Blerkom, 1999 |
| Cell cycle parameters | 8/20 | 40% | Present study and |
| that predict blastocyst | | | Wong/Loewke et al. |
| formation | | | 2010 |
| Cell cycle parameters | 7/11 | 63.6% | Present study |
| that predict normal CGH | | | |
| Cell cycle parameters | 7/11 | 63.6% | Present study |
| that predict normal CGH | | | |
| plus fragmentation (high | | | |
| degree) | | | |
| Cell cycle parameters | 7/8 | 87.5% | Present study |
| that predict normal CGH | | | |
| plus fragmentation | | | |
| (high & low degree) | | | |

TABLE 5
Probability of embryonic euploidy versus aneuploidy with single or multiple embryo transfer. A table demonstrating the
probability of embryonic euploidy and aneuploidy in a scenario, whereby 1, 2 or 3 embryos were selected for patient transfer based on
no or different morphological and/or parameter screening. The percentages in the table correspond to FIG. 12. Note that the
calculated values of embryonic euploidy with high fragmentation screening for 1, 2 or 3 embryos are congruent with observed values
as reported percentages of both single and multi-fetus pregnancies (cdc.gov/art) and based on our parameter and fragmentation
analysis, suggest that the transfer of multiple embryos may not be necessary to achieve IVF success.

| # of | | Fragmentation | | Cell Cycle Parameters that |
|---|---|---|---|---|
| Embryos | No Morphological | Assessment in Current | Cell Cycle | Predict Normal CGH Plus |
| Trans- | or Parameter | Clinical Practice | Parameters that | Fragmentation Assessment |
| ferred | Screening | (High Degree) | Predict Normal CGH | (High and Low Degree) |
| 1 | Euploid = 17.8% | Euploid = 26 7% | Euploid = 63.6% | Euploid = 87.5% |
| 2 | Both Euploid = 2.8% | Both Euploid = 6.7% | Both Euploid = 38.2% | Both Euploid = 76.1% |
| | Euploid/1 Aneuploid = 14.9% Both | 1 Euploid/1 Aneuploid = 20.0% | 1 Euploid/1 Aneuploid = 25.4% | 1 Euploid/1 Aneuploid = 11.4% |
| | Aneuploid = 67.3% | Both Aneuploid = 53.3% | Both Aneuploid = 10.9% | Both Aneuploid = 1.1% |
| 3 | All Euploid = 0.4% | All Euploid = 1.6% | All Euploid = 21.2% | All Euploid = 65.7% |
| | 2 Euploid/1 Aneuploid = 2.4% | 2 Euploid/1 Aneuploid = 5.1% | 2 Euploid/1 Aneuploid = 17.0% | 2 Euploid/1 Aneuploid = 10.4% |
| | 1 Euploid/2 Aneuploid = 12.5% | 1 Euploid/2 Aneuploid = 14.9% | 1 Euploid/2 Aneuploid = 8.5% | 1 Euploid/2 Aneuploid = 1.0% |
| | All Aneuploid = 54.7% | All Aneuploid = 38.4% | All Aneuploid = 2.4% | All Aneuploid = 0.05% |

TABLE 6
Several aneuploid embryos also exhibit sub-chromosomal losses and
gains. A table depicting the sub-chromosomal analysis of certain
low/high mitotic mosaic and triploid embryos with cell cycle parameter
timing illustrated in FIG. 11. Note that one of these embryos had a
balanced translocation between blastomeres and it is the only
embryo that did not exhibit cellular fragmentation.

| Embryo | Aneuploidy | Sub-Chromosomal | Fragmentation |
|---|---|---|---|
| Number | Type | Analysis | Observed |
| 1 | Low mitotic | Unbalanced partial gain Ch.10p | Yes |
| | mosaicism | | |
| 2 | Low mitotic | Balanced partial loss/gain Ch.1q | No |
| | mosaicism | | |
| 3 | High mitotic | Unbalanced partial loss Ch.10q | Yes |
| | mosaicism | | |
| 4 | Low mitotic | Unbalanced partial loss Ch.1q, | Yes |
| | mosaicsim | 10q and 16q and partial gain | |
| | | Ch.9q | |
| 5 | High mitotic | Unbalanced partial loss Ch.8p, | Yes |
| | mosaicism | 1q and 12q | |
| 6 | High mitotic | Unbalanced partial loss Ch.9q | Yes |
| | mosaicism | and partial gain Ch.1q, 7p, | |
| | | 10q and 16q | |
| 7 | Low mitotic | Unbalanced partial gain Ch.11q | Yes |
| | mosaicsim | | |
| 8 | Low mitotic | Unbalanced partial loss Ch.6q | Yes |
| | mosaicism | and 7p | |
| 9 | Triploid | Unbalanced partial gain Ch.22q | Yes |
| 10 | Triploid | Unbalanced partial gain Ch.19p | Yes |

Acknowledgement: This invention was made with support under contract RB3-02209 awarded by the California Institute for Regenerative Medicine.

While the preferred embodiments of the invention have been illustrated and described, it will be appreciated that various changes can be made therein without departing from the spirit and scope of the invention.

What is claimed is:

1. A method for evaluating the developmental potential of an embryo, the method comprising:
a) observing the embryo during development from the 1-cell stage to the 4-cell stage optically, wherein observations of the embryo are made periodically at time intervals of no more than 30 minutes; and
b) detecting embryonic micronuclei or cellular fragmentation if present, wherein the timing of the appearance of the embryonic micronuclei or cellular fragmentation is correlated with the developmental potential of the embryo.

2. The method of claim 1, wherein the appearance of embryonic micronuclei or cellular fragmentation at the 1-cell stage is correlated with the likelihood of embryonic lethality arising from meiotic chromosomal errors.

3. The method of claim 1, wherein the appearance of embryonic micronuclei or cellular fragmentation at the 2-cell stage, 3-cell stage, or 4-cell stage is correlated with the likelihood of embryonic lethality arising from mitotic chromosomal errors.

4. The method of claim 1, wherein micronuclei are detected by brightfield image analysis.

5. The method of claim 1, wherein the embryo is observed by time-lapse microscopy.

6. The method of claim 1, wherein the embryo is observed every 1 to 15 minutes.

7. The method of claim 1, wherein the embryo is observed every 5 minutes.

8. The method of claim 1, where Lamin-B1 immunostaining is used to detect embryonic micronuclei.

9. The method of claim 1, further comprising quantitating blastomere fragmentation.

10. The method of claim 1, further comprising detecting blastomere asymmetry.

11. The method of claim 1, further comprising evaluating the morphology of the embryo in culture at days 2, 3, 4, 5, or 6, or any combination thereof.

12. The method of claim 1, further comprising measuring one or more cell cycle parameters.

13. The method of claim 12, wherein one or more cell cycle parameters are selected from the group consisting of:
a) time between first and second mitosis,
b) time between second and third mitosis,
c) the duration of the first cytokinesis,
d) the time interval between cytokinesis 1 and cytokinesis 2, and
e) the time interval between cytokinesis 2 and cytokinesis 3.

14. The method of claim 1, further comprising measuring gene expression levels of one or more genes in the human embryo.

15. The method of claim 14, wherein one or more genes are selected from the group consisting of Cofillin, DIAPH1, ECT2, MYLC2/MYL5, DGCR8, Dicer/DICER1, TARBP2, CPEB1, Symplekin/SYMPK, YBX2, ZAR1, CTNNB1, DNMT3B, TERT, YY1, IFGR2/IFNGR2, BTF3, and NELF.

16. The method of claim 15, further comprising comparing gene expression levels of one or more genes of the human embryo to gene expression levels of one or more genes of a reference embryo, wherein a lower level of the expression of one or more genes selected from the group consisting of Cofillin, DIAPH1, ECT2, MYLC2/MYL5, DGCR8, Dicer/DICER1, TARBP2, CPEB1, Symplekin/SYMPK, YBX2, ZAR1, CTNNB1, DNMT3B, TERT, YY1, IFGR2/IFNGR2, BTF3 and NELF in said embryo relative to said reference embryo is indicative of poor developmental potential.

17. A method of detecting aneuploidy in an embryo, the method comprising:
a) observing the embryo during development from the 1-cell stage to the 4-cell stage optically, wherein observations of the embryo are made periodically at time intervals of no more than 30 minutes; and
b) detecting embryonic micronuclei or cellular fragmentation if present, wherein the presence of embryonic micronuclei or cellular fragmentation indicates the embryo is aneuploid.

18. The method of claim 17, wherein the appearance of embryonic micronuclei or cellular fragmentation at the 1-cell stage indicates that the aneuploidy in the embryo is caused by meiotic chromosomal errors.

19. The method of claim 17, wherein the appearance of embryonic micronuclei or cellular fragmentation at the 2-cell stage, 3-cell stage, or 4-cell stage indicates that the aneuploidy in the embryo is caused by mitotic chromosomal errors.

20. A method of selecting a human embryo with favorable developmental potential for transfer to a female subject, the method comprising:
a) culturing one or more embryos under conditions suitable for embryonic development;
b) observing the embryo during development from the 1-cell stage to the 4-cell stage optically, wherein observations of the embryo are made periodically at time intervals of no more than 30 minutes; and
c) selecting an embryo for transfer to the female subject, wherein the embryo has no detectable embryonic micronuclei or cellular fragmentation at the 2-cell stage, 3-cell stage, or 4-cell stage in order to avoid transfer of an embryo with mitotic chromosomal errors.

21. The method of claim 20, wherein the embryo is observed every 1 to 15 minutes.

22. The method of claim 21, wherein the embryo is observed every 5 minutes.

23. The method of claim 20, wherein micronuclei are detected by brightfield image analysis.

24. A method of selecting a human embryo with favorable developmental potential for transfer to a female subject, the method comprising:
a) culturing one or more embryos under conditions suitable for embryonic development;
b) observing the embryo during development from the 1-cell stage to the 4-cell stage optically, wherein observations of the embryo are made periodically at time intervals of no more than 30 minutes; and
c) selecting an embryo for transfer to the female subject, wherein the embryo has no detectable embryonic micronuclei or cellular fragmentation at the 1-cell stage in order to avoid transfer of an embryo with meiotic chromosomal errors.

25. The method of claim 24, wherein the embryo is observed every 1 to 15 minutes.

26. The method of claim 25, wherein the embryo is observed every 5 minutes.

27. The method of claim 24, wherein micronuclei are detected by brightfield image analysis.
